(19) United States
(12) Patent Application Publication
(10) Pub. No.: US 2021/0179636 A1
(43) Pub. Date: Jun. 17, 2021

(54) ANTIBACTERIAL COMPOUNDS

(71) Applicant: Redx Pharma PLC, Alderley Edge (GB)

(72) Inventors: Ian Cooper, Alderley Edge (GB); Amanda Lyons, Alderley Edge (GB)

(21) Appl. No.: 16/074,853

(22) PCT Filed: Feb. 8, 2017
(86) PCT No.: PCT/GB2017/050317

§ 371 Date: Aug. 2, 2018

(30) Foreign Application Priority Data
Feb. 8, 2016 (GB) 1602234.5
Sep. 28, 2016 (GB) 1616456.8

(51) Int. Cl. C07D 519/00

(52) U.S. Cl. CPC C07D 519/00

(57) ABSTRACT

This invention relates to antibacterial drug compounds containing a bicyclic core, typically a bicycle in which one of the rings is an oxazolidinone. It also relates to pharmaceutical formulations of antibacterial drug compounds. It also relates to uses of the derivatives in treating bacterial infections and in methods of treating bacterial infections.

This invention relates to antibacterial drug compounds containing a bicyclic core, typically a bicycle in which one of the rings is an oxazolidinone. It also relates to pharmaceutical formulations of antibacterial drug compounds. It also relates to uses of the derivatives in treating bacterial infections and in methods of treating bacterial infections. The invention is also directed to antibacterial drug compounds which are capable of treating bacterial infections which are currently hard to treat with existing drug compounds. Such infections are frequently referred to as resistant strains.

The increasing occurrence of bacterial resistance to antibiotics is viewed by many as being one of the most serious threats to human health. Multidrug resistance has become common among some pathogens, e.g. Staphylococcus aureus, Streptococcus pneumoniae, Clostridium difficile and Pseudomonas aeruginosa. Of these, Staphylococcus aureus, a Gram-positive bacterium, is the most concerning due to its potency and its capacity to adapt to environmental conditions. Methicillin resistant Staphylococcus aureus (MRSA) is probably the most well-known group of resistant strains and has reached pandemic proportions. Of particular concern is the increasing incidence of ‘community acquired’ infections, i.e. those occurring in subjects with no prior hospital exposure.

While less widespread, antibiotic resistant Gram-negative strains, such as either Escherichia coli NDM-1 (New Delhi metallo-β-lactamase 1) or Klebsiella pneumoniae NDM-1, are also very difficult to treat. Frequently only expensive antibiotics such as vancomycin and colistin are effective against these strains.

The fluoroquinolone antibacterial family are synthetic broad-spectrum antibiotics. They were originally introduced to treat Gram-negative bacterial infections, but are also used for the treatment of Gram-positive strains. One problem with existing fluoroquinolones can be the negative side effects that may sometimes occur as a result of their use. In general, the common side-effects are mild to moderate but, on occasion, more serious adverse effects occur. Some of the serious side effects that occur, and which occur more commonly with fluoroquinolones than with other antibiotic drug classes, include central nervous system (CNS) toxicity and cardiotoxicity. In cases of acute overdose there may be renal failure and seizure. In addition, an increasing number of strains of MRSA are also resistant to fluoroquinolone antibiotics, in addition to β-lactam antibiotics such as methicillin. Gonorrhoea is a human sexually-transmitted infection (STI) caused by the Gram-negative bacterium Neisseria gonorrhoeae, a species of the genus Neisseria that also includes the pathogen N. meningitidis, which is one of the aetiological agents of meningitis. Untreated infection can result in a range of clinical complications including urethritis, dysuria, epididymitis, pelvic inflammatory disease, cervicitis, endometritis and even infertility and ectopic pregnancy. In rare cases, gonorrhoea can also spread to the blood to cause disseminated gonococcal infection that can manifest as arthritis, endocarditis or meningitis. Human immunodeficiency virus (HIV) is more readily-transmitted in individuals co-infected with gonorrhoea. Throughout the twentieth and twenty-first centuries gonorrhoea has been treated with a range of antibiotics. The sulfonamides were the first antibiotics used for the treatment of gonorrhoea, followed by penicillin, tetracycline and spectinomycin. In each case the development of resistance to these drugs by N. gonorrhoeae led to their use being discontinued. The fluoroquinolone antibiotics ciprofloxacin and ofloxacin were also historically recommended for the treatment of gonorrhoea. However, by 2007, fluoroquinolone resistance rates had reached 15% of gonococcal isolates and their use was abandoned. Current treatment recommendations comprise the cephalosporin antibiotics cefixime or ceftriaxone in combination with azithromycin or doxycycline. Resistance to cefixime and ceftriaxone has emerged in recent years. The CDC estimates that approximately 246,000 of the 820,000 gonococcal infections per year in the United States are drug-resistant (Antibiotic Resistance Threats in the United States, 2013, Centers for Disease Control and Prevention).

Another disease in which the development of resistance and multidrug resistance is of particular concern is tuberculosis (TB). From the 17^{th} century to the early-20^{th} century TB was one of the most common causes of death. The development of effective treatments and vaccinations during the mid-20^{th} century led to a sharp reduction in the number of deaths arising from the disease. TB is usually caused by Mycobacterium tuberculosis. Mycobacteria are aerobic bacteria and, as a result, tuberculosis infections most often develop in the lungs (pulmonary tuberculosis), although this is not always the case. Mycobacteria lack an outer cell membrane and as such they are often classified as Gram-positive bacteria, although they are in many ways atypical. They have a unique cell wall which provides protection against harsh conditions (e.g. acidic, oxidative) but also provides natural protection against many antibiotics. Other antibiotics, such as beta-lactams, are inactive against TB due to the intrinsic lack of activity of the compounds in the mycobacteria. Thus, a drug molecule may have excellent activity against other bacterial strains but no activity against wild-type TB. A number of TB-specific antibiotics have been developed, such as isoniazid, rifampicin, pyrazinamide and ethambutol and these are typically used in combination. Unfortunately, there is now increasing incidence of multidrug-resistant TB (MDR-TB). MDR-TB is the term typically used to refer to TB that has developed resistance to isoniazid and rifampicin. MDR-TB can also be resistant to fluoroquinolones and also to the so-called ‘second-line’ injectable anti-TB drugs: kanamycin, capreomycin and amikacin. Where a strain of TB is resistant to isoniazid and rifampicin as well as one fluoroquinolone and one of the injectable anti-TB drugs, it is known as extensively drug resistant (XDR-TB). MDR-TB and XDR-TB are often found in those who have been previously treated for TB, but these forms of TB are just as infectious as wild-type TB and the incidence of MDR-TB and XDR-TB around the world is increasing. According to a 2013 World Health Organisation report, infections arising from XDR-TB had at that time been identified in 84 different countries. There have even been some reports of strains of TB which were resistant to all drugs tested against them (so-called ‘totally drug resistant tuberculosis’, TDR-TB). The ‘second-line’ anti-TB drugs and other antibiotics typically used to treat resistant infections can have unfavourable side effects.

Bacterial resistance is also becoming a problem in the treatment of animals. Antibacterials find widespread use in industrial farming, e.g. to prevent mastitis in dairy cattle, where they are often used prophylactically. Such widespread prophylactic use has led to the build-up of resistance in certain bacterial strains that are particularly relevant to animal health.

In spite of the numerous different antibiotics known in the art for a variety of different infections, there continues to be a need for antibiotics that can provide an effective treatment in a reliable manner. In addition, there remains a need for antibiotic drugs that can avoid or reduce the side-effects associated with known antibiotics.

It is an aim of certain embodiments of this invention to provide new antibiotics. In particular, it is an aim of certain embodiments of this invention to provide antibiotics that are active against resistant strains of Gram-positive and/or Gram-negative bacteria. It is an aim of certain embodiments of this invention to provide compounds that have activity that is comparable to those of existing antibiotics, and ideally which is better. It is an aim of certain embodiments of this invention to provide such activity against wild-type strains at the same time as providing activity against one or more resistant strains.

It is an aim of certain embodiments of this invention to provide compounds that exhibit a smaller decrease in activity against resistant strains compared to wild-type strains than prior art compounds do. It may be that certain compounds of the invention are less active than prior art compounds but there is a benefit associated with having a more consistent activity against a range of strains.

It is an aim of certain embodiments of this invention to provide antibiotics that exhibit reduced cytotoxicity relative to prior art compounds and existing therapies.

It is an aim of certain embodiments of this invention to provide treatment of bacterial infections that is effective in a selective manner at a chosen site of interest. Another aim of certain embodiments of this invention is to provide antibiotics having a convenient pharmacokinetic profile and a suitable duration of action following dosing. A further aim of certain embodiments of this invention is to provide antibiotics in which the metabolised fragment or fragments of the drug after absorption are GRAS (Generally Regarded As Safe).

Certain embodiments of the present invention satisfy some or all of the above aims.

### Compounds of the Invention

In a first aspect, the invention provides a compound of formula (I), or a pharmaceutically acceptable salt or N-Oxide thereof:

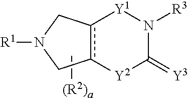

wherein [symbol] is a double bond or a single bond;

Y¹ is —CR⁴R⁴—

Y² is independently selected from O and S;

Y³ is independently selected from O and S;

R¹ is independently selected from -L¹-Ar¹-Ar² and

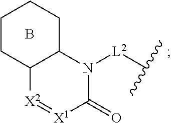

Ar¹ and Ar² are each independently selected from a phenyl or monocyclic heteroaryl group;

-L¹- is —C₁-C₃-alkylene-;

X¹ is independently selected from N and CR⁵ and X² is independently selected from N and CR⁶; or

X¹ and X² together form a 5-membered heteroaryl ring;

-L²- is —C₂-C₃-alkylene-;

Ring B is independently selected from: phenyl, monocyclic 6-membered heteroaryl and pyridinone, optionally substituted with a single —Y⁴—R⁷ group; Y⁴ is absent or is independently selected from NR⁸, O and S; where Ring B is a pyridinone ring, the nitrogen of the Ring B pyridinone may be attached to the proximal end of a —C₁-C₃-alkylene-group that is attached at its distal end to the group -L²-

R² is independently at each occurrence selected from: halo, nitro, cyano, NR⁹R¹⁰, NR⁹S(O)₂R⁹, NR⁹CONR⁹R⁹, NR⁹C(O)R⁹, NR⁹CO₂R⁹, OR⁹, SR⁹, SOR⁹, SO₃R⁹, SO₂R⁹, SO₂NR⁹R⁹, CO₂R⁹, C(O)R⁹, CONR⁹R⁹, C₁-C₄-alkyl, C₂-C₄-alkenyl, C₂-C₄-alkynyl, C₁-C₄-haloalkyl and O—C₁-C₄-haloalkyl;

R³ is a bicyclic carbocyclic or heterocyclic ring system in which at least one of the two rings is aryl or heteroaryl;

or R³ is -L³-phenyl; wherein -L³- is selected from —CR¹¹═CR¹¹— and —C₄-cycloalkyl-;

R⁴, R⁹ and R¹¹ are each independently at each occurrence selected from: H and C₁-C₄-alkyl;

R⁵ and R⁶ are each independently selected from H, halo, cyano, C₁-C₄-alkyl and O—C₁-C₄-alkyl;

R⁷ is independently selected from: H, C₁-C₄-alkyl, C₂-C₄-alkenyl, C₂-C₄-alkynyl, C₁-C₄-haloalkyl, C₃-C₃-cycloalkyl, ₄₋₇-heterocycloalkyl, phenyl, monocyclic heteroaryl and C₁-C₃-alkylene-R^{7a}; wherein R^{7a} is independently selected from C₃-C₃-cycloalkyl, ₄₋₇-heterocycloalkyl, phenyl and monocyclic heteroaryl;

R⁸ is independently selected from: H and C₁-C₄-alkyl;

or R⁷ and R⁸ together with the nitrogen to which they are attached form a 4- to 7-membered heterocycloalkyl ring;

R¹⁰ is independently at each occurrence selected from: H, C₁-C₄ alkyl, C₁-C₄-haloalkyl, S(O)₂—C₁-C₄-alkyl and C(O)—C₁-C₄-alkyl;

a is an integer from 0 to 4;

wherein any of the aforementioned alkyl, alkylene, alkenyl, alkynyl, haloalkyl, cycloalkyl, carbocyclic, heterocyclic, heterocycloalkyl, aryl, phenyl and heteroaryl groups is optionally substituted, where chemically possible, by 1 to 5 substituents which are each independently at each occurrence selected from the group consisting of: oxo, ═NR^{a}, ═NOR^{a}, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR¹, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl, C₁-C₄-haloalkyl and O—C₁-C₄-haloalkyl; wherein R^{a} is independently at each occurrence selected from H, C₁-C₄-alkyl.

For the absence of doubt, where ring B is substituted with a single —Y⁴—R⁷ group it may also be substituted with further substituents as described above, i.e. further substituents selected from selected from oxo, ═NR^{a}, ═NOR^{a}, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl, C₁-C₄-haloalkyl and O—C₁-C₄-haloalkyl.

In certain embodiments, the compound of formula (I) is a compound of formula (II):

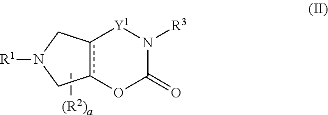

wherein [symbol] , R¹, R², R³, Y¹ and a are as defined above for formula (I).

In certain embodiments, the compound of formula (I) is a compound of formula (III):

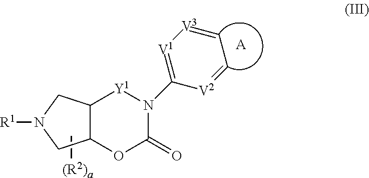

wherein R¹, R², Y¹ and a are as defined above for formula (I) and wherein V¹, V² and V³ are each independently selected from: N and CR¹²; with the proviso that no more than two of V¹, V² and V³ are N; and wherein the ring A is a substituted or unsubstituted 5- or 6-membered saturated cycloalkyl or heterocycloalkyl ring; and R¹² is independently at each occurrence selected from H, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄-haloalkyl.

In certain embodiments, the compound of formula (I) is a compound of formula (IV):

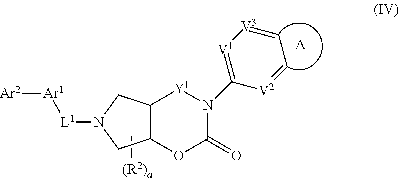

wherein Ar¹, Ar², R², L¹, Y¹ and a are as defined above for formula (I) and wherein V¹, V² and V³ and ring A are as described above for formula (III).

In certain embodiments, the compound of formula (I) is a compound of formula (V):

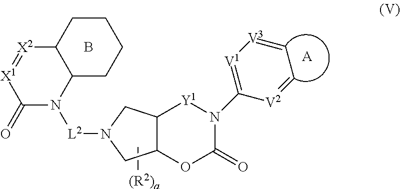

wherein R², L², Y¹, X¹, X², Ring B and a are as defined above for formula (I) and wherein V¹, V² and V³ and ring A are as described above for formula (III).

In certain embodiments, the compound of formula (I) is a compound of formula (VI):

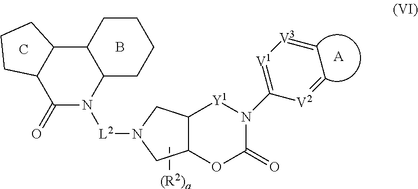

wherein R², L², Y¹, Ring B and a are as defined above for formula (I) and wherein V¹, V² and V³ and ring A are as described above for formula (III) and wherein ring C is a 5-membered heteroaryl ring.

In certain embodiments, the compound of formula (I) is a compound of formula (VII):

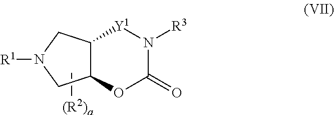

wherein R¹, R², R³, Y¹ and a are as defined above for formula (I). For the absence of doubt the hashed and solid wedges in formula (VII) are intended to depict the relative stereochemistry of the indicated bonds and not the absolute stereochemistry, i.e. the compound may be in the form of a single enantiomer or in the form of a racemate.

In certain embodiments, the compound of formula (I) is a compound of formula (VIII):

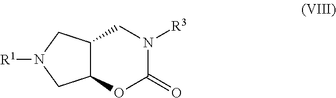

wherein R¹ and R³ are as defined above for formula (I). For the absence of doubt the hashed and solid wedges in formula (VIII) are intended to depict the relative stereochemistry of the indicated bonds and not the absolute stereochemistry, i.e. the compound may be in the form of a single enantiomer or in the form of a racemate.

In certain embodiments, the compound of formula (I) is a compound of formula (IX):

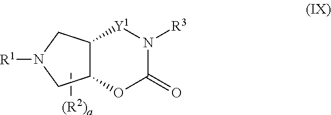

wherein R¹, R², R³, Y¹ and a are as defined above for formula (I). For the absence of doubt the hashed and solid wedges in formula (IX) are intended to depict the relative stereochemistry of the indicated bonds and not the absolute stereochemistry, i.e. the compound may be in the form of a single enantiomer or in the form of a racemate.

In an embodiment, the compound of formula (I) is a compound of formula (X):

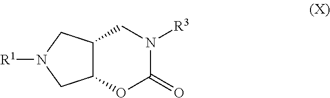

wherein R¹ and R³ are as defined above for formula (I). For the absence of doubt the hashed and solid wedges in formula (X) are intended to depict the relative stereochemistry of the indicated bonds and not the absolute stereochemistry, i.e. the compound may be in the form of a single enantiomer or in the form of a racemate.

The following statements apply to compounds of any of formulae (I) to (X). These statements are independent and interchangeable. In other words, any of the features described in any one of the following statements may (where chemically allowable) be combined with the features described in one or more other statements below. In particular, where a compound is exemplified or illustrated in this specification, any two or more of the statements below which describe a feature of that compound, expressed at any level of generality, may be combined so as to represent subject matter which is contemplated as forming part of the disclosure of this invention in this specification. [symbol] may be a double bond. Typically, however, [symbol] is a single bond.

Where [symbol] is a single bond, it may be that the groups Y¹ and Y² are orientated cis to each other. Alternatively, it may be that the groups Y¹ and Y² are orientated trans to each other.

R⁴ may be at one occurrence C₁-C₄-alkyl, e.g. C₁-C₂-alkyl. R⁴ may at the other occurrence be H. R⁴ may at each occurrence be C₁-C₂-alkyl. Preferably, however, R⁴ is at each occurrence H. Thus, Y¹ is preferably —CH₂—.

Y² may be S. Preferably, however, Y² is O.

Y³ may be S. Preferably, however, Y³ is O.

R¹ may be:

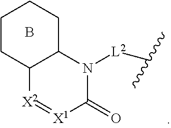

Throughout this specification, the group

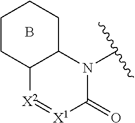

may be referred to as R^{1a}. Thus, R¹ may be -L²-R^{1a}.

-L²- may be —C₂-alkylene-. -L²- may be —C₃-alkylene-. -L²- may be substituted with 1 or 2 groups selected from ═O, methyl, CH₂OH, CO₂R^{a}, and CO₂NR^{a}R^{a}. It may be, however, that -L²- is unsubstituted alkylene. Thus, -L¹- may be —CH₂CH₂—. Alternatively, -L¹- may be —CH₂CH₂CH₂—.

X¹ may be N. Thus, R^{1a} may have the structure:

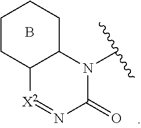

Alternatively, X¹ may be CR⁵. Thus, R^{1a} may have the structure:

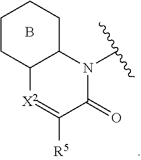

X² may be N. Thus, R^{1a} may have the structure:

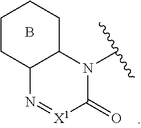

Alternatively, X² may be CR⁶. Thus, R¹ may have the structure:

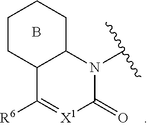

It may be that X¹ is N and X² is CR. Thus, R^{1a} may have the structure:

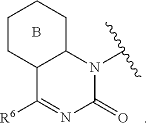

It may be that X¹ is N and X² is N. Thus, R^{1a} may have the structure:

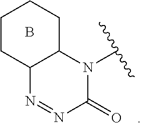

It may be that X¹ is CR⁵ and X² is N. Thus, R^{1a} may have the structure:

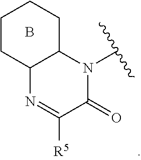

It may be that X¹ is CR⁵ and X² is CR⁶. Thus, R^{1a} may have the structure:

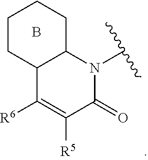

It may be that R⁵ and R⁶ are each independently selected from H, halo, cyano, C₁-C₄-alkyl and O—C₁-C₄-alkyl; or R⁵ and R⁶ together with the carbons to which they are attached together form a 5-membered heteroaryl ring. Compounds in which R⁵ and R⁶ together with the carbons to which they are attached together form a 5-membered heteroaryl ring are examples of compounds is which X¹ and X² together form a 5-membered heteroaryl group.

It may be that R⁵ is independently selected from H or C₁-C₄-alkyl. It may be that R⁵ is H. It may be that R⁶ is independently selected from H or C₁-C₄-alkyl. It may be that R⁶ is H.

It may be that R⁵ and R⁶ are each independently selected from: H, halo, cyano, C₁-C₄-alkyl and O—C₁-C₄-alkyl. It may be that R⁶ and R⁵ are each independently selected from H, C₁-C₄-alkyl and O—C₁-C₄-alkyl or that R⁶ and R⁵, together with the carbons to which they are attached together form a 5-membered heteroaryl ring. It may be that R⁵ and R⁶ are each independently at each occurrence selected from H or C₁-C₄-alkyl. It may be that R⁵ and R⁶ are at each occurrence H.

Alternatively, it may be that R⁵ and R⁶, together with the carbons to which they are attached together form a 5-membered heteroaryl ring. Exemplary heteroaryl rings include oxazole, thiazole, isoxazole, isothiazole, pyrazole, imidazole, triazole, pyrole, thiophene, furan and oxadiazole. For the absence of doubt, the double bond depicted in the structure above between X¹ and X² may be delocalised into the heteroaromatic ring.

Thus, R^{1a} may have the structure:

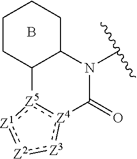

wherein Z⁴ and Z⁵ are each independently selected from C and N; Z¹, Z² and Z³ are each independently selected from O, S, N, S(O), NR^{a} and CR¹³; wherein the ring formed by Z¹, Z², Z³, Z⁴ and Z⁵ contains two endocyclic double bonds and with the further proviso that at least one of Z¹, Z², Z³, Z⁴ and Z⁵ is O, S, N or NR^{a}; and wherein R¹³ is independently selected from H, C₁-C₄-alkyl, CR^{a}R^{a}OR^{a}, CR^{a}R^{a}NR^{a}R^{a}, CO₂R^{a} and CONR^{a}R^{a}.

In certain examples, the heteroaryl ring may be a ring selected from oxazole, thiazole, isoxazole and isothiazole. Thus, R^{1a} may have the structure:

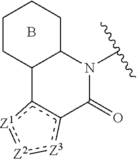

wherein one of Z¹, Z² and Z³ is N, one of Z¹, Z² and Z³ is CR¹³ and the final one of Z¹, Z² and Z³ is selected from O and S; provided that the ring comprising Z¹, Z² and Z³ contains two endocyclic double bonds; and wherein R¹³ is independently selected from H, C₁-C₄-alkyl, CR^{a}R^{a}OR^{a}, CR^{a}R^{a}NR^{a}R^{a}, CO₂R^{a} and CONR^{a}R^{a}.

Thus, R^{1a} may have the structure:

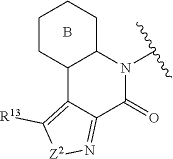

wherein Z² is independently selected from O and S. Z² may be O. Z² may be S. Ring B may be selected from a phenyl ring or a 6-membered heteroaryl ring. Thus, Ring B may be a phenyl ring. Ring B may be a pyridyl ring.

R^{1a} may have the structure:

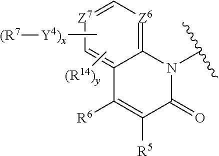

wherein x is 0 or 1; y is an integer from 0 to 2; Z⁶ and Z⁷ are each independently selected from carbon or nitrogen; and R¹⁴ is independently selected from halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄-haloalkyl.

Thus, R^{1a} may have the structure:

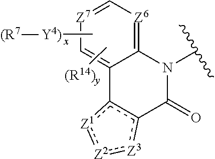

wherein one of Z¹, Z² and Z³ is N, one of Z¹, Z² and Z³ is CR¹³ and the final one of Z¹, Z² and Z³ is selected from O and S; provided that the ring comprising Z¹, Z² and Z³ contains two endocyclic double bonds.

Likewise, R^{1a} may have the structure:

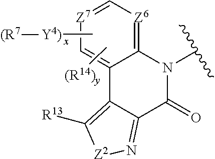

wherein Z² is independently selected from O and S. Z² may be O. Z² may be S.

x may be 0. Thus it may be that there is no Y⁴—R⁷ group on R¹.

Alternatively, x may be 1.

Thus, R^{1a} may have the structure:

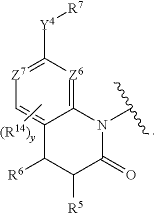

R^{1a} may have the structure:

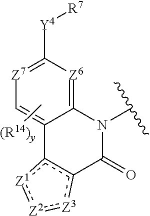

wherein one of Z¹, Z² and Z³ is N, one of Z¹, Z² and Z³ is CR¹³ and the final one of Z¹, Z² and Z³ is selected from O and S; provided that the ring comprising Z¹, Z² and Z³ contains two endocyclic double bonds.

Likewise, R^{1a} may have the structure:

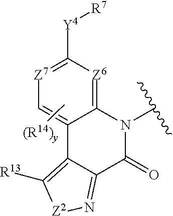

wherein Z² is independently selected from O and S. Z² may be O. Z² may be S.

R^{1a} may have the structure:

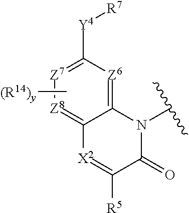

wherein y is an integer from 0 to 2; Z⁶, Z⁷ and Z⁸ are each independently selected from carbon or nitrogen; providing that no more than 2 of Z⁶, Z⁷ and Z⁸ are nitrogen; and R¹⁴ is independently selected from halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl, C₁-C₄-haloalkyl and O—C₁-C₄-haloalkyl.

y may be 0.

Z⁶ may be nitrogen. Alternatively, Z⁶ may be carbon. Z⁷ may be nitrogen. Alternatively, Z⁷ may be carbon. It may be that Z⁶ and Z⁷ are each carbon. It may be that Z⁶ and Z⁷ are each nitrogen. It may be that Z⁶ is nitrogen and Z⁷ is carbon.

Z⁸ may be nitrogen. Alternatively, Z⁸ may be carbon.

R¹³ may be selected from H and C₁-C₄-alkyl. In certain particular embodiments, R¹³ is H.

If present, R¹⁴ may at each occurrence be selected from halo and C₁-C₄-alkyl.

Y⁴ may be independently selected from NR⁸, O and S. Y⁴ is preferably O.

It may be that R⁷ and R⁸ together with the nitrogen to which they are attached form a 4- to 7-membered heterocycloalkyl ring. It may be that R⁷ and R⁸ together with the nitrogen to which they are attached form a 6-membered heterocycloalkyl ring, e.g. a piperidine, morpholine or piperazine ring.

Preferably, however, R⁷ is independently selected from: H, C₁-C₄-alkyl, C₃-C₈-cycloalkyl, ₄₋₇-heterocycloalkyl, phenyl and monocyclic heteroaryl. R⁷ may be independently selected from: C₁-C₄-alkyl, phenyl and monocyclic heteroaryl.

R⁷ may be alkyl. R⁷ may be selected from C₁-C₄-alkyl. R⁷ may be alkyl (e.g. C₁-C₄-alkyl) substituted with an NR^{a}R^{a} group or with a nitrogen containing 5- to 7-membered heterocycloalkyl group.

Alternatively, R⁷ may be selected from phenyl and monocyclic heteroaryl. R⁷ may be phenyl. R⁷ may be unsubstituted phenyl or R⁷ may be substituted phenyl. R⁷ may be monocyclic heteroaryl, e.g. a 6-membered heteroaryl group. Thus, R⁷ may be pyridyl, e.g. unsubstituted pyridyl. R⁷ may be 3-pyridyl, e.g. unsubstituted 3-pyridyl. R⁷ may be C₃-C₈-cycloalkyl, e.g. cyclohexyl. R⁷ may be ₄₋₇-heterocycloalkyl, e.g. piperidine or tetrahydropyran.

It may be that Y⁴ is O and R⁷ is independently selected from: C₁-C₄-alkyl, C₂-C₄-alkenyl, C₂-C₄-alkynyl, C₁-C₄-haloalkyl, C₃-C₈-cycloalkyl, ₄₋₇-heterocycloalkyl, phenyl, monocyclic heteroaryl and C₁-C₃-alkylene-R^{7a}; wherein R⁷ is independently selected from C₃-C₈-cycloalkyl, ₄₋₇-heterocycloalkyl, phenyl and monocyclic heteroaryl.

Exemplary R^{1a} groups include:

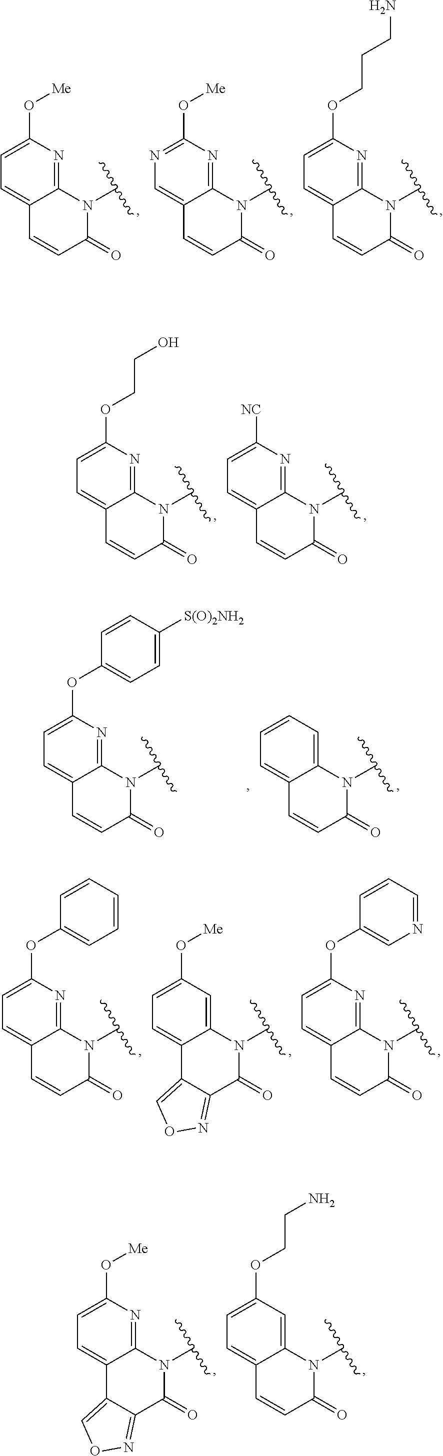

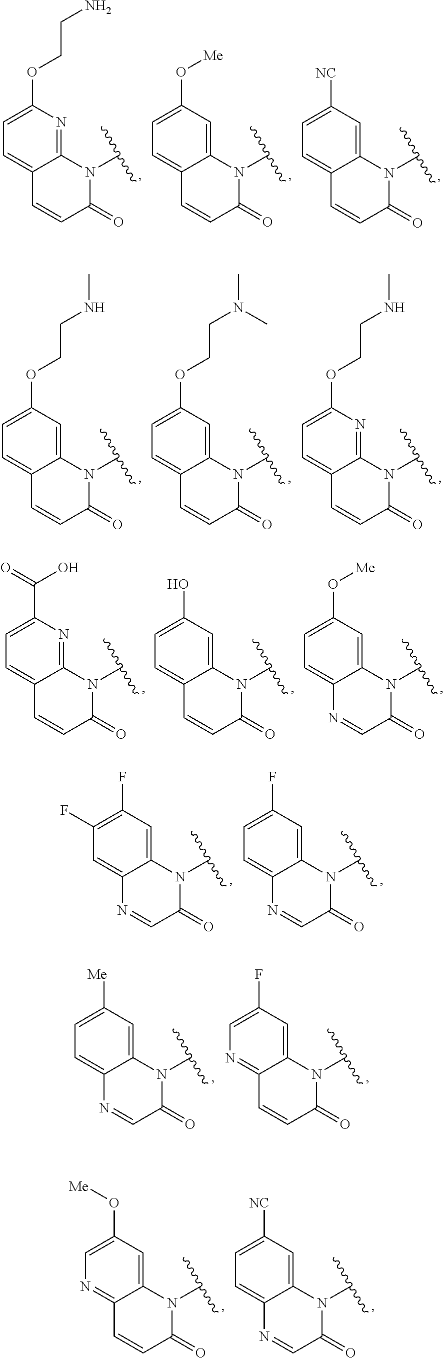

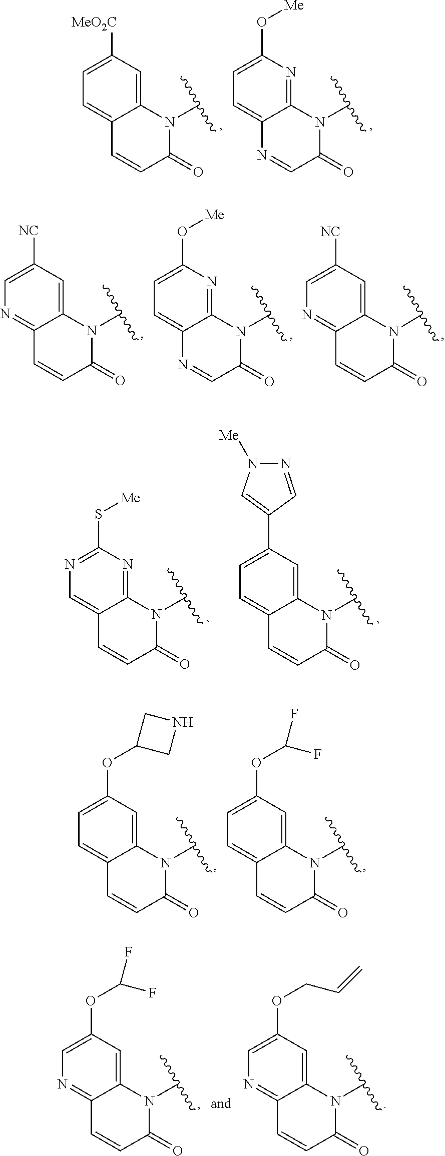

In these embodiments, -L²- is typically an ethylene group. -L²- may also be a propylene group.

It may be that R¹ is -L-Ar¹-Ar²; wherein Ar¹ is independently selected from a phenyl or monocyclic heteroaryl group; and wherein Ar² is a monocyclic heteroaryl group. -L¹- may be —C₁-C₂-alkylene-. -L¹- may be —C-alkylene-. -L¹- may be —C₂-alkylene-. -L¹- may be substituted with 1 or 2 groups selected from ═O, methyl, CH₂OH, CO₂R^{a}, and CO₂NR^{a}R^{a}. It may be, however, that -L¹- is unsubstituted alkylene. Thus, -L¹- may be —CH₂—.

It may be that -L¹- and R⁴ together with the nitrogen to which they are attached form a 4- to 7-membered heterocyclic ring. Thus, it may be that -L¹- and R⁴ together with the nitrogen to which they are attached form a 4- to 5-membered heterocyclic ring. -L¹- and R⁴ together with the nitrogen to which they are attached may form a pyrrolidine or azetidine ring.

It may be that at least one of Ar¹ and Ar² is a monocyclic heteroaryl group, e.g. a 6-membered monocyclic heteroaryl group, e.g. a pyridine. It may be that a single one of Ar¹ and Ar² is a monocyclic heteroaryl group, e.g. a 6-membered monocyclic heteroaryl group, e.g. a pyridine. It may be that a single one of Ar¹ and Ar² is phenyl, e.g. substituted phenyl.

It may be that Ar¹ is a phenyl group, e.g. a substituted phenyl group and Ar² is a 6-membered heteroaryl group, e.g. pyridine. It may be that Ar¹ is a 6-membered heteroaryl group, e.g. pyridine, and Ar² is a phenyl group.

Ar¹ may be a monocyclic heteroaryl group, e.g. a 6-membered monocyclic heteroaryl group. Ar¹ may be pyridine. Ar¹ may be a phenyl group. Ar¹ may be unsubstituted. Ar¹ may be substituted, e.g. Ar¹ may be substituted with a single hydroxyl group.

Thus, Ar¹ may have the structure:

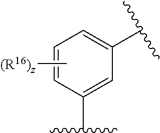

wherein z is an integer from 0 to 4; and R¹⁶ is independently selected from halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄ haloalkyl. Ar¹ may have the structure:

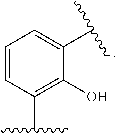 [SMILES: *c1cccc(*)c1O]

Ar² may be a monocyclic heteroaryl group. Ar² may be 6-membered heteroaryl group, e.g. a pyridyl group.

Ar² may have the structure:

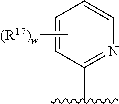

wherein w is an integer from 0 to 4; and R¹⁷ is independently selected from halo, nitro, cyano, NR^{a}R^{a}, NR^{a}S(O)₂R^{a}, NRC(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄-haloalkyl. Ar¹ may have the structure:

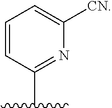 [SMILES: *c1cccc(C#N)n1]

Exemplary examples of Ar¹-Ar² include:

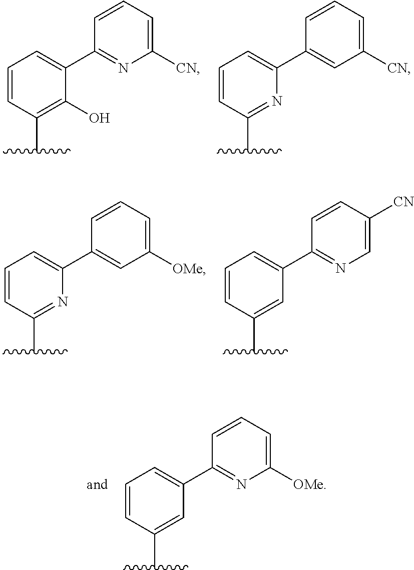

In these embodiments, -L¹- is typically a methylene group.

R² may be independently at each occurrence selected from: CO₂R⁹, C(O)R⁹, CONR⁹R⁹, C₁-C₄-alkyl, C₂-C₄-alkenyl, C₂-C₄-alkynyl and C₁-C₄-haloalkyl. a may be 0.

It may be that R³ is a bicyclic carbocyclic or heterocyclic ring system in which at least one of the two rings is aryl or heteroaryl.

Thus, R³ may take the form:

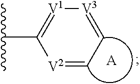

wherein V¹, V² and V³ are each independently selected from: N and CR¹²; with the proviso that no more than two of V¹, V² and V³ are N; and wherein the ring A is a substituted or unsubstituted 5- or 6-membered saturated cycloalkyl or heterocycloalkyl ring; and wherein R¹² is independently at each occurrence selected from H, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄-haloalkyl.

Preferably, R³ takes the form:

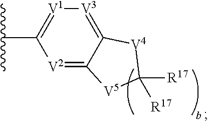

wherein V⁴ and V⁵ are each independently selected from O, S and NR^{a}; R¹⁷ is independently at each occurrence selected from: H, fluoro, cyano, CO₂R^{a}, C(O)R^{a}, CONR^{a}R^{a}, C₁-C₄-alkyl, C₂-C₄-alkenyl, C₂-C₄-alkynyl and C₁-C₄-haloalkyl; or any two R¹⁷ groups which are attached to the same carbon together form a group selected from: ═O, ═NR^{a} and ═NOR^{a}; and b is an integer selected from 1 and 2. For the absence of doubt, V¹, V², V, V⁴, V, R¹⁷ and b are selected such that the number of substituent groups (as defined above in relation to formula (I)) off the R³ bicycle does not exceed 5.

It may be that V¹, V² and V³ are each independently selected from: N and CH; with the proviso that no more than two of V¹, V² and V³ are N. It may be that a single one of V¹, V² and V³ is N. Preferably, V³ is CR¹² (e.g. CH). Thus, it may be that V¹ is N and V² is CR¹² (e.g. CH). Alternatively, it may be that V² is N and V¹ is CR¹² (e.g. CH). In a further alternative, it may be that V¹ and V² are each N.

R¹⁷ may be independently at each occurrence selected from: H, fluoro, cyano, CO₂R^{a}, C(O)R^{a}, CONR^{a}R^{a}, C₁-C₄-alkyl, C₂-C₄-alkenyl, C₂-C₄-alkynyl and C₁-C₄-haloalkyl; or any two R¹⁷ groups which are attached to the same carbon together form a group selected from: ═O, ═NR^{a} and ═NOR^{a}. Preferably R¹⁷ is independently at each occurrence selected from H, F, C₁-C₄-alkyl or C₁-C₄-haloalkyl; or any two R¹⁷ groups which are attached to the same carbon together form a ═O group. Preferably R¹⁷ is independently at each occurrence selected from: H, C₁-C₄-alkyl or C₁-C₄-haloalkyl; or any two R¹⁷ groups which are attached to the same carbon together form a ═O group.

In a preferred embodiment, V⁴ is O. Thus, it may be that both V⁴ and V⁵ are O. It may be that V⁴ is O and V⁵ is S. It may be that V⁴ is O and V⁵ is NR^{a} (e.g. NH).

V⁴ can also be S. Thus, it may be that V⁴ is S and V⁵ is NR^{a} (e.g. NH).

It may be that V⁵ is NR^{a} (e.g. NH). In this case it is preferable that the —CR¹⁷R¹⁷— group attached to said V⁵ is C═O. b may be 1. Preferably, b is 2.

In a specific embodiment, V⁴ is O, V⁵ is O, b is 2 and R¹⁷ is at each occurrence H. In another specific embodiment, V⁴ is O, V⁵ is S, b is 2 and R¹⁷ is at each occurrence H. In yet another specific embodiment, V⁴ is O, V⁵ is NH, b is 2, the —CR¹⁷R¹⁷— group attached to V⁵ is C═O and the —CR¹⁷R¹⁷— group attached to V⁴ is CH₂.

R³ may take the form:

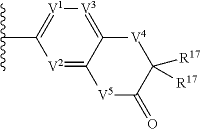

wherein V¹, V² and V³ are each independently selected from: N and CR¹²; with the proviso that no more than two of V¹, V² and V³ are N; V⁴ and V⁵ are each independently selected from O, S and NR^{a}; wherein R¹² is independently at each occurrence selected from H, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄-haloalkyl; and R¹⁷ is independently at each occurrence selected from: H, fluoro, cyano, CO₂R^{a}, C(O)R^{a}, CONR^{a}R^{a}, C₁-C₄-alkyl, C₂-C₄-alkenyl, C₂-C₄-alkynyl and C₁-C₄-haloalkyl.

R³ may take the form:

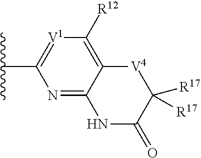

wherein V¹ is are each independently selected from: N and CR¹⁰; V⁴ is independently selected from O and S; wherein R¹⁰ is independently at each occurrence selected from H, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄-haloalkyl; and R¹⁵ is independently at each occurrence selected from: H, fluoro, cyano, CO₂R^{a}, C(O)R^{a}, CONR^{a}R^{a}, C₁-C₄-alkyl, C₂-C₄-alkenyl, C₂-C₄-alkynyl and C₁-C₄-haloalkyl.

Exemplary R³ groups include:

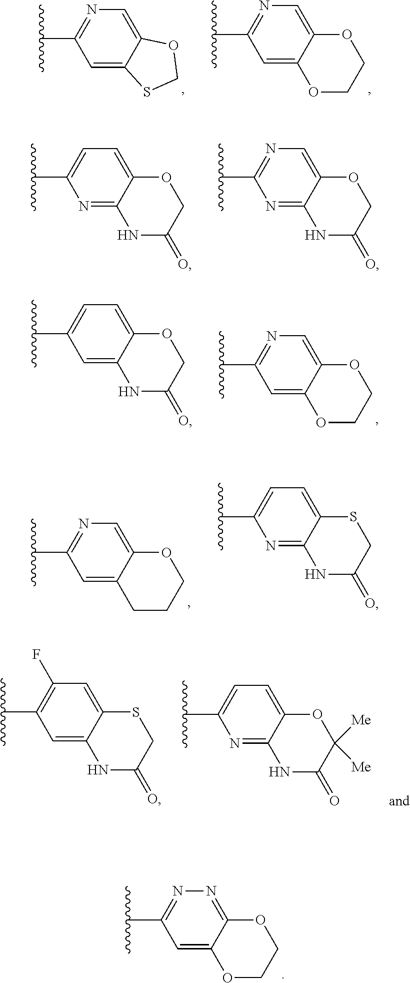

A preferred R³ group is:

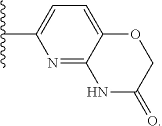 [SMILES: *c1ccc2c(n1)NC(=O)CO2]

R³ may be -L-phenyl.

R³ may take the form:

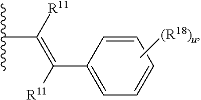

wherein R¹⁸ is independently at each occurrence selected from halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄-haloalkyl; and u is an integer from 0 to 5.

R¹¹ may at each occurrence be H.

R³ may take the form:

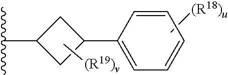

wherein R¹⁸ is independently at each occurrence selected from halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄-haloalkyl; R¹⁹ is independently at each occurrence selected from oxo, fluoro, cyano, CO₂R^{a} C(O)R^{a}, CONR^{a}R^{a}, C₁-C₄-alkyl, C₂-C₄-alkenyl, C₂-C₄-alkynyl and C₁-C₄-haloalkyl; u is an integer from 0 to 5; and v is an integer from 0 to 4;

R¹⁹ may be selected from fluoro, cyano, CO₂R^{a}, C(O)R^{a}, CONR^{a}R^{a}, C₁-C₄-alkyl, C₂-C₄-alkenyl, C₂-C₄-alkynyl and C₁-C₄-haloalkyl.

v may be 0.

R¹⁸ may be independently at each occurrence selected from C₁-C₄-alkyl, halo, nitro and cyano.

u may be an integer from 1 to 5, e.g. from 1 to 3.

R³ may also take the form

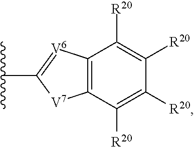

wherein V⁶ is independently selected from N and CR¹² (e.g. CH); V⁷ is independently selected from NR^{a}, S and O; and R²⁰ is independently at each occurrence selected from: H, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, NR^{a}C(O)R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, C₁-C₄-alkyl, C₂-C₄-alkenyl, C₂-C₄-alkynyl, C₁-C₄-haloalkyl, and CR^{a}R^{a}NR^{a}R^{a}. R²⁰ may be independently at each occurrence selected from H, F, CN, OR^{a}, nitro, C₁-C₄-alkyl, C₂-C₄-alkenyl, C₂-C₄-alkynyl and C₁-C₄-haloalkyl. For the absence of doubt, V⁶, V⁷ and R²⁰ are selected such that the number of substituent groups (as defined above in relation to formula (I)) off the R³ bicycle does not exceed 5.

R³ may take the form

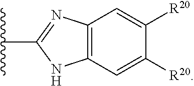

An exemplary R³ group is

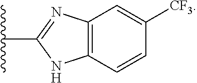 [SMILES: *c1nc2cc(C(F)(F)F)ccc2[nH]1]

The compound may be any one or more compound(s) selected from those made in Examples 1 to 17 and/or tested in Examples 18 or 19, or a pharmaceutically acceptable salt or N-oxide thereof.

The compound may be as described in any of the following numbered clauses:

1. A compound of formula (I), or a pharmaceutically acceptable salt or N-oxide thereof:

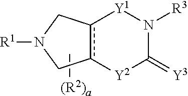

wherein [symbol] is a double bond or a single bond;

Y¹ is —CR⁴R⁴—

Y² is independently selected from O and S;

Y³ is independently selected from O and S;

R¹ is independently selected from -L-Ar¹-Ar² and

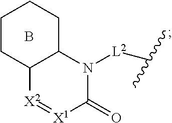

Ar¹ is independently selected from a phenyl or monocyclic heteroaryl group;

Ar² is a monocyclic heteroaryl group;

-L¹- is —C₁-C₃-alkylene-;

X¹ is independently selected from N and CR⁵ and X² is independently selected from N and CR⁶; or

X¹ and X² together form a 5-membered heteroaryl ring;

-L²- is —C₂-C₃-alkylene-;

Ring B is independently selected from: phenyl, monocyclic 6-membered heteroaryl and pyridinone, optionally substituted with a single —Y⁴—R⁷ group; Y⁴ is independently selected from NR⁸, O and S; where Ring B is a pyridinone ring, the nitrogen of the Ring B pyridinone may be attached to the proximal end of a —C₁-C₃-alkylene- group that is attached at its distal end to the group -L²-

R² is independently at each occurrence selected from: halo, nitro, cyano, NR⁹R¹⁰, NR⁹S(O)₂R⁹, NR⁹CONR⁹R⁹, NR⁹C(O)R⁹, NR⁹CO₂R⁹, OR⁹, SR⁹, SOR⁹, SO₃R⁹, SO₂R⁹, SO₂NR⁹R⁹, CO₂R⁹, C(O)R⁹, CONR⁹R⁹, C₁-C₄-alkyl, C₂-C₄-alkenyl, C₂-C₄-alkynyl and C₁-C₄-haloalkyl;

R³ is a bicyclic carbocyclic or heterocyclic ring system in which at least one of the two rings is aryl or heteroaryl;

or R³ is -L³-phenyl; wherein -L³- is selected from —CR¹¹═CR¹¹— and —C₄-cycloalkyl-;

R⁴, R⁹ and R¹¹ are each independently at each occurrence selected from: H and C₁-C₄-alkyl;

R⁵ and R⁶ are each independently selected from H, halo, cyano, C₁-C₄-alkyl and O—C₁-C₄-alkyl;

R⁷ is independently selected from: H, C₁-C₄-alkyl, C₃-C₈-cycloalkyl, ₄₋₇-heterocycloalkyl, phenyl and monocyclic heteroaryl;

R⁸ is independently selected from: H and C₁-C₄-alkyl;

or R⁷ and R⁸ together with the nitrogen to which they are attached form a 4- to 7-membered heterocycloalkyl ring;

R¹⁰ is independently at each occurrence selected from: H, C₁-C₄ alkyl, C₁-C₄-haloalkyl, S(O)₂—C₁-C₄-alkyl and C(O)—C₁-C₄-alkyl;

a is an integer from 0 to 4;

wherein any of the aforementioned alkyl, alkylene, alkenyl, alkynyl, haloalkyl, cycloalkyl, carbocyclic, heterocyclic, heterocycloalkyl, aryl, phenyl and heteroaryl groups is optionally substituted, where chemically possible, by 1 to 5 substituents which are each independently at each occurrence selected from the group consisting of: oxo, ═NR^{a}, ═NOR^{a}, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄-haloalkyl; wherein R^{a} is independently at each occurrence selected from H, C₁-C₄-alkyl.

2. A compound of clause 1, wherein the compound of formula (I) is a compound of formula (VII):

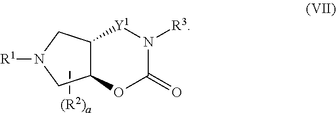

3. A compound of clause 1 or clause 2, wherein a is O.

4. A compound of any preceding clause, wherein R¹ is:

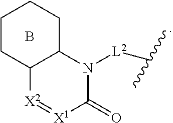

5. A compound of clause 4, wherein -L²- is —C₂-alkylene-.

6. A compound of clause 4 or clause 5, wherein X¹ is CR⁵ and X² is CR⁶.

7. A compound of any one of clauses 4 to 5, wherein R⁵ and R⁶, together with the carbons to which they are attached together form a 5-membered heteroaryl ring.

8. A compound of clause 7, wherein the heteroaryl ring is a ring selected from oxazole, thiazole, isoxazole and isothiazole.

9. A compound of any one of clauses 4 to 8, wherein Ring B is a phenyl ring.

10. A compound of any one of clauses 4 to 8, wherein Ring B is a pyridine ring or a pyrimidine ring.

11. A compound of any one of clauses 1 to 3, wherein R¹ is -L-Ar¹-Ar²; wherein Ar¹ is independently selected from a phenyl or monocyclic heteroaryl group; and wherein Ar² is a monocyclic heteroaryl group.

12. A compound of clause 11, wherein -L¹. is —C-alkylene-.

13. A compound of clause 12, wherein Ar¹ is a phenyl group.

14. A compound of clause 12 or clause 13, wherein Ar² is a 6-membered heteroaryl group.

15. A compound of any preceding clause, wherein R³ takes the form:

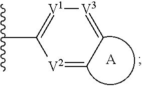

wherein V¹, V² and V³ are each independently selected from: N and CR¹²; with the proviso that no more than two of V¹, V² and V³ are N; and wherein the ring A is a substituted or unsubstituted 5- or 6-membered saturated cycloalkyl or heterocycloalkyl ring; and wherein R¹² is independently at each occurrence selected from H, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄-haloalkyl.

16. A compound of any preceding clause, wherein Y¹ is —CH₂—.

### DETAILED DESCRIPTION

Throughout this specification, the term ‘compound of the invention’ is intended to refer to a compound of any one of formulae (I) to (X) or a pharmaceutically acceptable salt or N-oxide thereof.

Where the compound of the invention is an N-oxide, it will typically be a pyridine N-oxide, i.e. where the compound of the invention comprises a pyridine ring (which may form part of a bicyclic or tricyclic ring system), the nitrogen of that pyridine may be N⁺—O⁻. Alternatively, it may be that the compound of the invention is not an N-oxide.

Included within the scope of the present invention are all stereoisomers, geometric isomers and tautomeric forms of the compounds of the invention, including compounds exhibiting more than one type of isomerism, and mixtures of one or more thereof. Also included are acid addition or base salts wherein the counter ion is optically active, for example, d-lactate or l-lysine, or racemic, for example, dl-tartrate or dl-arginine.

Compounds of the invention containing one or more asymmetric carbon atoms can exist as two or more stereoisomers. Where a compound of the invention contains a double bond such as a C═C or C═N group, geometric cis/trans (or Z/E) isomers are possible. Specifically, the oxime groups present in certain compounds of the invention may be present as the E-oxime, as the Z-oxime or as a mixture of both in any proportion. Cis/trans isomers may be separated by conventional techniques well known to those skilled in the art, for example, chromatography and fractional crystallisation.

Where structurally isomeric forms of a compound are interconvertible via a low energy barrier, tautomeric isomerism (‘tautomerism’) can occur. This can take the form of proton tautomerism in compounds of the invention containing, for example, an imino, keto, or oxime group, or so-called valence tautomerism in compounds which contain an aromatic moiety.

Conventional techniques for the preparation/isolation of individual enantiomers when necessary include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of the invention contains an acidic or basic moiety, a base or acid such as 1-phenylethylamine or tartaric acid. The resulting diastereomeric mixture may be separated by chromatography and/or fractional crystallization and one or both of the diastereoisomers converted into the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, “Stereochemistry of Organic Compounds” by E. L. Eliel and S. H. Wilen (Wiley, 1994).

It follows that a single compound may exhibit more than one type of isomerism.

The term Cₘ-Cₙ refers to a group with m to n carbon atoms.

The term “alkyl” refers to a monovalent linear or branched hydrocarbon chain. For example, C₁-C₆-alkyl may refer to methyl, ethyl, n-propyl, iso-propyl, n-butyl, sec-butyl, tert-butyl, n-pentyl and n-hexyl. An alkyl group may be unsubstituted or substituted by one or more substituents. Specific substituents for each alkyl group independently may be fluorine, OR^{a} or NHR^{a}.

The term “alkylene” refers to a bivalent linear hydrocarbon chain. For example, —C₁-C₃-alkyl may refer to methylene, ethylene or propylene. An alkylene group may be unsubstituted or substituted by one or more substituents. Specific substituents for each alkyl group independently may be methyl or ethyl.

The term “haloalkyl” refers to a hydrocarbon chain substituted with at least one halogen atom independently chosen at each occurrence from: fluorine, chlorine, bromine and iodine. The halogen atom may be present at any position on the hydrocarbon chain. For example, C₁-C₆-haloalkyl may refer to chloromethyl, fluoromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl. A halo alkyl group may be a fluoroalkyl group, i.e. a hydrocarbon chain substituted with at least one halogen atom.

The term “alkenyl” refers to a branched or linear hydrocarbon chain containing at least one double bond. The double bond(s) may be present as the E or Z isomer. The double bond may be at any possible position of the hydrocarbon chain. For example, “C₂-C₆-alkenyl” may refer to ethenyl, propenyl, butenyl, butadienyl, pentenyl, pentadienyl, hexenyl and hexadienyl. An alkenyl group may be unsubstituted or substituted by one or more substituents. Specific substituents for any saturated carbon atom in each alkenyl group independently may be fluorine, OR^{a} or NHR^{a}.

The term “alkynyl” refers to a branched or linear hydrocarbon chain containing at least one triple bond. The triple bond may be at any possible position of the hydrocarbon chain. For example, “C₂-C₆-alkynyl” may refer to ethynyl, propynyl, butynyl, pentynyl and hexynyl. An alkynyl group may be unsubstituted or substituted by one or more substituents. Specific substituents for any saturated carbon atom in each alkynyl group independently may be fluorine, OR^{a} or NHR^{a}.

The term “cycloalkyl” refers to a saturated hydrocarbon ring system containing 3, 4, 5 or 6 carbon atoms. For example, “C₃-C₆-cycloalkyl” may refer to cyclopropyl, cyclobutyl, cyclopentyl, cyclohexyl. A cycloalkyl group may be unsubstituted or substituted by one or more substituents. Specific substituents for each cycloalkyl group independently may be fluorine, OR^{a} or NHR^{a}.

The term “aromatic” when applied to a substituent as a whole means a single ring or polycyclic ring system with 4n+2 electrons in a conjugated π system within the ring or ring system where all atoms contributing to the conjugated π system are in the same plane.

The term “heteroaromatic” when applied to a substituent as a whole means a single ring or polycyclic ring system with 4n+2 electrons in a conjugated π system within the ring or ring system where all atoms contributing to the conjugated π system are in the same plane, the ring system comprising from 1 to 4 heteroatoms independently selected from O, S and N (in other words from 1 to 4 of the atoms forming the ring or ring system are selected from O, S and N).

The term “aryl” refers to an aromatic hydrocarbon ring system. The ring system has 4n+2 electrons in a conjugated π system within a ring where all atoms contributing to the conjugated π system are in the same plane. For example, the “aryl” may be phenyl and naphthyl. The aryl group may be unsubstituted or substituted by one or more substituents.

Specific substituents for each aryl group independently may be C₁-C₄-alkyl, C₁-C₄-haloalkyl, cyano, halogen, OR^{a} or NHR^{a}.

Aryl groups may have from 6 to 20 carbon atoms as appropriate to satisfy valency requirements. Aryl groups comprise aromatic rings, i.e. rings which satisfy the Huckel rule. Aryl groups may be optionally substituted phenyl groups, optionally substituted biphenyl groups, optionally substituted naphthalenyl groups or optionally substituted anthracenyl groups. Equally, aryl groups may include non-aromatic carbocyclic portions. An aromatic ring is a phenyl ring.

The term “heteroaryl” may refer to any aromatic (i.e. a ring system containing (4n+2) π-electrons or n-electrons in the π-system) 5-10 membered ring system comprising from 1 to 4 heteroatoms independently selected from O, S and N (in other words from 1 to 4 of the atoms forming the ring system are selected from O, S and N). Thus, any heteroaryl groups may be independently selected from: 5 membered heteroaryl groups in which the heteroaromatic ring is substituted with 1-4 heteroatoms independently selected from O, S and N; and 6-membered heteroaryl groups in which the heteroaromatic ring is substituted with 1-3 (e.g. 1-2) nitrogen atoms; 9-membered bicyclic heteroaryl groups in which the heteroaromatic system is substituted with 1-4 heteroatoms independently selected from O, S and N; 10-membered bicyclic heteroaryl groups in which the heteroaromatic system is substituted with 1-4 nitrogen atoms. Specifically, heteroaryl groups may be independently selected from: pyrrole, furan, thiophene, pyrazole, imidazole, oxazole, isoxazole, triazole, oxadiazole, thiadiazole, tetrazole; pyridine, pyridazine, pyrimidine, pyrazine, triazine, indole, isoindole, benzofuran, isobenzofuran, benzothiophene, indazole, benzimidazole, benzoxazole, benzthiazole, benzisoxazole, purine, quinoline, isoquinoline, cinnoline, quinazoline, quinoxaline, pteridine, phthalazine, naphthyridine. Heteroaryl groups may also be 6-membered heteroaryl groups in which the heteroaromatic ring is substituted with 1 heteroatomic group independently selected from O, S and NH and the ring also comprises a carbonyl group. Such groups include pyridones and pyranones. The heteroaryl system itself may be substituted with other groups. A heteroaryl group may be unsubstituted or substituted by one or more substituents. Specific substituents for each heteroaryl group independently may be C₁-C₄-alkyl, C₁-C₄-haloalkyl, cyano, halogen, OR^{a} or NHR^{a}.

Heteroaryl groups may mean a 5- or 6-membered heteroaryl group. They may therefore comprise a 5- or 6-membered heteroaromatic ring, i.e. a 5- or 6-membered ring which satisfies the Huckel rule and comprises a heteroatom. Heteroaryl groups may be selected from: 5-membered heteroaryl groups in which the heteroaromatic ring is includes 1-4 heteroatoms selected from O, S and N; and 6-membered heteroaryl groups in which the heteroaromatic ring includes 1-2 nitrogen atoms. Specifically, heteroaryl groups and heteroaromatic rings may be selected from: pyrrole, furan, thiophene, pyrazole, imidazole, oxazole, isoxazole, triazole, oxadiazole, thiodiazole, pyridine, pyridazine, pyrimidine, pyrazine.

The term “_{y-z}-membered heterocycloalkyl” may refer to a monocyclic or bicyclic saturated or partially saturated group having from y to z atoms in the ring system and comprising 1 or 2 heteroatoms independently selected from O, S and N in the ring system (in other words 1 or 2 of the atoms forming the ring system are selected from O, S and N). By partially saturated it is meant that the ring may comprise one or two double bonds. This applies particularly to monocyclic rings with from 5 to 8 members. The double bond will typically be between two carbon atoms but may be between a carbon atom and a nitrogen atom. Examples of heterocycloalkyl groups include; piperidine, piperazine, morpholine, thiomorpholine, pyrrolidine, tetrahydrofuran, tetrahydrothiophene, dihydrofuran, tetrahydropyran, dihydropyran, dioxane, azepine. Bicyclic systems may be spiro-fused, i.e. where the rings are linked to each other through a single carbon atom; vicinally fused, i.e. where the rings are linked to each other through two adjacent carbon or nitrogen atoms; or they may be share a bridgehead, i.e. the rings are linked to each other two non-adjacent carbon or nitrogen atoms. The heterocycloalkyl groups may be unsubstituted or substituted by one or more substituents. Specific substituents for any saturated carbon atom in each heterocycloalkyl group may independently be fluorine, OR^{a} or NHR^{a}.

An ‘endocyclic’ double bond is one where both of the atoms between which the double bond is formed are in the ring or ring system in which the atoms are situated.

A carbocyclic group consists of one or more rings which are entirely formed from carbon atoms. A carbocylic group can be a mono- or bicyclic cycloalkyl group, or it can comprise at least one phenyl ring.

A heterocyclic group consists of one or more rings wherein the ring system includes at least one heteroatom. A heterocyclic group comprises at least one heteroaryl or heterocycloalkyl rings. A heterocycloalkyl ring may be a saturated ring comprising at least one heteroatom selected from O, S and N.

Where a ring system is described as being a x-membered bicyclic group, that is intended to mean that the skeleton of the bicyclic ring system is formed from x atoms (i.e. the total number of atoms across the two rings of the bicycle is x).

Aryl and heteroaryl groups are optionally substituted with 1 to 5 substituents which are each independently at each occurrence selected from the group consisting of: halo, nitro, cyano, NR^{a}R^{a}, NR^{a}S(O)₂R^{a}, NR^{a}CONR^{a}R^{a}, NR^{a}CO₂R^{a}, NR^{a}C(O)R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, C₁-C₄-alkyl, C₂-C₄-alkenyl, C₂-C₄-alkynyl, C₁-C₄-haloalkyl and CR^{a}R^{a}NR^{a}R^{a}; wherein R^{a} is independently at each occurrence selected from H, C₁-C₄-alkyl and C₁-C₄-haloalkyl.

The present invention also includes the synthesis of all pharmaceutically acceptable isotopically-labelled compounds of formulae (I) to (X) wherein one or more atoms are replaced by atoms having the same atomic number, but an atomic mass or mass number different from the atomic mass or mass number most commonly found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled compounds, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as C ¹⁸F, ¹⁵O and ¹³N, can be useful in Positron Emission Topography (PET) studies for examining substrate receptor occupancy.

Isotopically-labelled compounds can generally be prepared by conventional techniques known to those skilled in the art or by processes analogous to those described using an appropriate isotopically-labelled reagent in place of the non-labelled reagent previously employed.

### Uses, Methods of Treatment and Pharmaceutical Formulations

Each of the compounds of the present invention may be used as a medicament. Thus, in another aspect of the invention, there is provided a compound as defined above for the treatment of bacterial infections.

The compounds and formulations of the present invention may be used in the treatment of a wide range of bacterial infections. In some embodiments, the compounds can be used to treat bacterial infections caused by one or more resistant strains of bacteria e.g. a strain which is resistant to at least one approved antibiotic drug. In a further embodiment, the compounds can be used to treat bacterial infections caused by one or more resistant strains of Gram-positive bacteria e.g a strain which is resistant to at least one approved antibiotic drug. In a further embodiment, the compounds can be used to treat bacterial infections caused by one or more resistant strains of Gram-negative bacteria, e.g. a strain which is resistant to at least one approved antibiotic drug.

The compounds and formulations of the invention may be used to treat infections caused by bacteria which are in the form of a biofilm.

The term ‘resistant strains’ is intended to mean strains of bacteria which have shown resistance to one or more known antibacterial drug. For example, it may refer to strains which are resistant to methicillin, strains that are resistant to one or more other β-lactam antibiotics, strains that are resistant to one or more fluoroquinolones and/or strains that are resistant to one or more other antibiotics (i.e. antibiotics other than β-lactams and fluoroquinolones). A resistant strain is one in which the MIC of a given compound or class of compounds for that strain has shifted to a significantly higher number than for the parent (susceptible) strain.

The term ‘approved drug’ is intended to mean that the drug is one which had been approved by the US FDA or the EMA prior to 1 Feb. 2016.

The bacterial strain (e.g. the MRSA strain or E. coli strain) may be resistant to one or more fluoroquinolone antibiotics, e.g. one or more antibiotics selected from levofloxacin, enoxacin, fleroxacin, lomefloxacin, nadifloxacin, norfloxacin, rufloxacin, balofloxacin, grepafloxacin, pazufloxacin, sparfloxacin, temafloxacin, tosufloxacin, besifloxacin, clinafloxacin, garenoxacin, gemifloxacin, gatifloxacin, sitafloxacin, trovafloxacin, prulifloxacin, ciprofloxacin, pefloxacin, moxifloxacin, ofloxacin, delafloxacin, zabofloxacin, avarofloxacin, finafloxacin.

The compounds of the invention may be particularly effective at treating infections caused by Gram-positive bacteria. The compounds of the invention may be particularly effective at treating infections caused by Gram-positive bacteria which are resistant to one or more fluoroquinolone antibiotics.

The compounds of the invention may be particularly effective at treating infections caused by Gram-negative bacteria. The compounds of the invention may be particularly effective at treating infections caused by Gram-negative bacteria which are resistant to one or more fluoroquinolone antibiotics.

The compounds of the invention may be particularly effective at treating infections caused by aerobic bacteria, e.g. S. aureus. The compounds of the invention may be particularly effective at treating infections caused by anaerobic bacteria, e.g. a Clostridium spp. such as Clostridium difficile.

The compounds and formulations of the present invention can be used to treat or to prevent infections caused by bacterial strains associated with biowarfare. These may be strains which are category A pathogens as identified by the US government (e.g. those which cause anthrax, plague etc.) and/or they may be strains which are category B pathogens as identified by the US government (e.g. those which cause Glanders disease, mellioidosis etc). In a specific embodiment, the compounds and formulations of the present invention can be used to treat or to prevent infections caused by Gram-positive bacterial strains associated with biowarfare (e.g. anthrax). More particularly, the compounds and formulations may be used to treat category A and/or category B pathogens as defined by the US government on 1 Jan. 2014.

The bacterial infection may be caused by a strain selected from: Neisseria spp., Haemophilus spp., Legionella spp., Pasteurella spp., Bordetella spp., Brucella spp., Francisella spp. and Moraxella spp. Like Neisseria spp., Haemophilus spp., Legionella spp., Pasteurella spp., Bordetella spp., Brucella spp., Francisella spp. and Moraxella spp. are fastidious Gram-negative organisms. A fastidious bacterium is one having a complex nutritional requirement, i.e. one which will only grow when specific nutrients are included in the culture medium. As an example Neisseria gonorrhoeae requires, amongst other supplements, iron, several amino acids, cofactors and vitamins in order to grow. Members of the fastidious Gram-negative bacteria group often share common antibiotic susceptibility profiles. Pathogenic Neisseria species include Neisseria gonorrhoeae (the pathogen responsible for gonorrhoea) and Neisseria meningitidis (one of the pathogens responsible for bacterial meningitis). Infections which can be treated by the compounds and methods of the invention include gonorrhoea. Infections which can be treated include secondary infections which can arise from lack of treatment of a primary Neisseria gonorrhoeae infection. Exemplary secondary infections include urethritis, dysuria, epididymitis, pelvic inflammatory disease, cervicitis and endometritis and also systemic gonococcal infections (e.g. those manifesting as arthritis, endocarditis or meningitis). The gonorrhoea infection may be one caused by a strain of Neisseria gonorrhoeae which is resistant to at least one known antibacterial drug, e.g. at least one β-lactam drug. The gonorrhoea infection may be one caused by a strain of Neisseria gonorrhoeae which is resistant to at least one approved drug. The at least one drug may be an antibiotic drug, e.g. one that is approved for use in treating one of the fastidious Gram-negative species mentioned in this specification. It may be approved for use in treating gonorrhoea. The approved drug may be a β-lactam drug. Further infections which can be treated by the compounds and methods of the invention include bacterial meningitis and Neisseria meningitidis infections of other parts of the human or animal body.

The compounds of the invention can be used to treat or prevent mycobacterial infections, e.g. mycobacterial infections caused by resistant strains of mycobacteria. Thus, for example, they can be used to treat TB or leprosy. Thus, it may be that the mycobacterial infection is caused by M. tuberculosis. It may also be that the mycobacterial infection is caused by a mycobacterium selected from: M. avium complex, M. abscessus, M. leprae, M. bovis, M. kansasii, M. chelonae, M. africanum, M. canetti and M. microti. The compounds may be used to treat resistant strains of TB, e.g. MDR-TB (i.e. TB infections caused by strains which are resistant to isoniazid and rifampicin), XDR-TB (i.e. TB infections caused by strains which are resistant to isoniazid, rifampicin, at least one fluoroquinolone and at least one of kanamycin, capreomycin and amikacin) and/or TDR-TB (i.e. TB infections caused by strains which have proved resistant to every drug tested against it with the exception of a compound of the invention). The mycobacterium is caused by a mycobacterial strain which is resistant to at least one approved antimycobacterial compound. The at least one approved antimycobacterial compound may be selected from: rifampicin, isoniazid, kanamycin, capreomycin, amikacin and a fluoroquinolone. The at least one approved antimycobacterial compound may be selected from: rifampicin, moxifloxacin, isoniazid, ciprofloxacin and levofloxacin. The compounds of the invention may be used to treat non-replicating TB.

The compounds of the invention may also be useful in treating other forms of infectious disease, e.g. fungal infections, parasitic infections and/or viral infections.

The compounds of the present invention can be used in the treatment of the human body. They may be used in the treatment of the animal body. In particular, the compounds of the present invention can be used to treat commercial animals such as livestock. Alternatively, the compounds of the present invention can be used to treat companion animals such as cats, dogs, etc.

The compounds of the invention may be obtained, stored and/or administered in the form of a pharmaceutically acceptable salt. Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulfonic, toluenesulfonic, benzenesulfonic, salicylic, sulfanilic, aspartic, glutamic, edetic, stearic, palmitic, oleic, lauric, pantothenic, tannic, ascorbic and valeric acids. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts. Also included are acid addition or base salts wherein the counter ion is optically active, for example, d-lactate or l-lysine, or racemic, for example, dl-tartrate or dl-arginine.

Compounds of the invention may exist in a single crystal form or in a mixture of crystal forms or they may be amorphous. Thus, compounds of the invention intended for pharmaceutical use may be administered as crystalline or amorphous products. They may be obtained, for example, as solid plugs, powders, or films by methods such as precipitation, crystallization, freeze drying, or spray drying, or evaporative drying. Microwave or radio frequency drying may be used for this purpose.

For the above-mentioned compounds of the invention the dosage administered will, of course, vary with the compound employed, the mode of administration, the treatment desired and the disorder indicated. For example, if the compound of the invention is administered orally, then the daily dosage of the compound of the invention may be in the range from 0.01 micrograms per kilogram body weight (μg/kg) to 100 milligrams per kilogram body weight (mg/kg).

A compound of the invention, or pharmaceutically acceptable salt thereof, may be used on their own but will generally be administered in the form of a pharmaceutical composition in which the compounds of the invention, or pharmaceutically acceptable salt thereof, is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

The compounds of the invention may be administered in combination with other active compounds (e.g. antifungal compounds, oncology compounds) and, in particular, with other antibacterial compounds. The compound of the invention and the other active (e.g. the other antibacterial compound) may be administered in different pharmaceutical formulations either simultaneously or sequentially with the other active. Alternatively, the compound of the invention and the other active (e.g. the other antibacterial compound) may form part of the same pharmaceutical formulation.

Examples of other antibacterial compounds which could be administered with the compounds of the invention are penems, carbapenems, fluoroquinolones, β-lactams, vancomycin, erythromycin or any other known antibiotic drug molecule.

Depending on the mode of administration of the compounds of the invention, the pharmaceutical composition which is used to administer the compounds of the invention will preferably comprise from 0.05 to 99% w (percent by weight) compounds of the invention, more preferably from 0.05 to 80% w compounds of the invention, still more preferably from 0.10 to 70% w compounds of the invention, and even more preferably from 0.10 to 50% w compounds of the invention, all percentages by weight being based on total composition.

The pharmaceutical compositions may be administered topically (e.g. to the skin) in the form, e.g., of creams, gels, lotions, solutions, suspensions, or systemically, e.g. by oral administration in the form of tablets, capsules, syrups, powders, suspensions, solutions or granules; or by parenteral administration in the form of a sterile solution, suspension or emulsion for injection (including intravenous, subcutaneous, intramuscular, intravascular or infusion); or by rectal administration in the form of suppositories; or by inhalation (i.e. in the form of an aerosol or by nebulisation).

If administered topically, high-dosages of the compounds of the invention can be administered. Thus, a compound with an in vitro MIC of, for example, 16-64 μg/mL may still provide an effective treatment against certain bacterial infections.

For oral administration the compounds of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compounds of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules. Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, sweetening agents (such as saccharine), preservative agents and/or carboxymethylcellulose as a thickening agent or other excipients known to those skilled in the art.

For intravenous (parenteral) administration the compounds of the invention may be administered as a sterile aqueous or oily solution.

The size of the dose for therapeutic purposes of compounds of the invention will naturally vary according to the nature and severity of the conditions, the age and sex of the animal or patient and the route of administration, according to well-known principles of medicine.

Dosage levels, dose frequency, and treatment durations of compounds of the invention are expected to differ depending on the formulation and clinical indication, age, and co-morbid medical conditions of the patient. The standard duration of treatment with compounds of the invention is expected to vary between one and seven days for most clinical indications. It may be necessary to extend the duration of treatment beyond seven days in instances of recurrent infections or infections associated with tissues or implanted materials to which there is poor blood supply including bones/joints, respiratory tract, endocardium, and dental tissues.

In another aspect the present invention provides a pharmaceutical formulation comprising a compound of the invention and a pharmaceutically acceptable excipient. The formulation may further comprise one or more other antibiotics, e.g. one or more fluoroquinolone antibiotics. Illustrative fluoroquinolone antibiotics include levofloxacin, enoxacin, fleroxacin, lomefloxacin, nadifloxacin, norfloxacin, rufloxacin, balofloxacin, grepafloxacin, pazufloxacin, sparfloxacin, temafloxacin, tosufloxacin, besifloxacin, clinafloxacin, garenoxacin, gemifloxacin, gatifloxacin, sitafloxacin, trovafloxacin, prulifloxacin, ciprofloxacin, pefloxacin, moxifloxacin, ofloxacin, delafloxacin, zabofloxacin, avarofloxacin, finafloxacin.

In another aspect of the invention is provided a method of treating a bacterial infection, the method comprising treating a subject in need thereof with a therapeutically effective amount of a compound of the invention.

### Medical Uses

The compounds of the present invention can be used in the treatment of the human body.

The compounds of the invention may be for use in treating human bacterial infections such as infections of the genitourinary system, the respiratory tract, the gastrointestinal tract, the ear, the skin, the throat, soft tissue, bone and joints (including infections caused by Staphylococcus aureus). The compounds can be used to treat pneumonia, sinusitis, acute bacterial sinusitis, bronchitis, acute bacterial exacerbation of chronic bronchitis, anthrax, chronic bacterial prostatitis, acute pyelonephritis, pharyngitis, tuberculosis, tonsillitis, Escherichia coli, prophylaxis before dental surgery, cellulitis, acnes, cystitis, infectious diarrhoea, typhoid fever, infections caused by anaerobic bacteria, peritonitis, abdominal infection, bacteraemia, septicaemia, sexually transmitted bacterial infection (e.g. gonorrhoea, Chlamydia), bacterial vaginosis, pelvic inflammatory disease, pseudomembranous colitis, Helicobacter pylori, acute gingivitis, Crohn's disease, rosacea, fungating tumours, impetigo.

The compounds of the present invention may also be used in treating other conditions treatable by eliminating or reducing a bacterial infection. In this case they will act in a secondary manner alongside for example a chemotherapeutic agent used in the treatment of cancer.

In yet another aspect of the invention is provided the use of a compound of the invention for the preparation of a medicament. The medicament may be for use in the treatment of any of the diseases, infections and indications mentioned in this specification.

In an aspect of the invention is provided a compound of the invention for medical use. The compound may be used in the treatment of any of the diseases, infections and indications mentioned in this specification.

### Veterinary Uses

They may be used in the treatment of the animal body. In particular, the compounds of the present invention can be used to treat commercial animals such as livestock. The livestock may be mammal (excluding humans) e.g. cows, pigs, goats, sheep, llamas, alpacas, camels and rabbits. The livestock may be birds (e.g. chickens, turkeys, ducks, geese etc.). Alternatively, the compounds of the present invention can be used to treat companion animals such as cats, dogs, etc. The veterinary use may be to treat wild populations of animals in order to prevent the spread of disease to humans or to commercial animals. In this case, the animals may be rats, badgers, deer, foxes, wolves, mice, kangaroos and monkeys and other apes.

In an aspect of the invention is provided a compound of the invention for veterinary use. The compound may be used in the treatment of any of the animal diseases and infections and indications mentioned in this specification.

In another aspect the present invention provides a veterinary formulation comprising a compound of the invention and a veterinarily acceptable excipient.

The methods by which the compounds may be administered for veterinary use include oral administration by capsule, bolus, tablet or drench, topical administration as an ointment, a pour-on, spot-on, dip, spray, mousse, shampoo, collar or powder formulation or, alternatively, they can be administered by injection (e.g. subcutaneously, intramuscularly or intravenously), or as an implant. Such formulations may be prepared in a conventional manner in accordance with standard veterinary practice. The formulations will vary with regard to the weight of active compound contained therein, depending on the species of animal to be treated, the severity and type of infection and the body weight of the animal. For parenteral, topical and oral administration, typical dose ranges of the active ingredient are 0.01 to 100 mg per kg of body weight of the animal. Preferably the range is 0.1 to 10 mg per kg. In any event, the veterinary practitioner, or the skilled person, will be able to determine the actual dosage which will be most suitable for an individual patient, which may vary with the species, age, weight and response of the particular patient. The above dosages are exemplary of the average case; there can, of course, be individual instances where higher or lower dosage ranges are merited, and such are within the scope of this invention.

As an alternative, when treating animals the compounds may be administered with the animal feedstuff and for this purpose a concentrated feed additive or premix may be prepared for mixing with the normal animal feed.

Certain compounds of the invention are of particular use in the treatment of mastitis. In this regard, a particularly preferred method of administration is by injection into the udder of a subject (e.g. a cow, a goat, a pig or sheep).

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### Synthesis

The skilled man will appreciate that adaptation of methods known in the art could be applied in the manufacture of the compounds of the present invention.

For example, the skilled person will be immediately familiar with standard textbooks such as “Comprehensive Organic Transformations—A Guide to Functional Group Transformations”, RC Larock, Wiley-VCH (1999 or later editions), “March's Advanced Organic Chemistry -Reactions, Mechanisms and Structure”, MB Smith, J. March, Wiley, (5th edition or later) “Advanced Organic Chemistry, Part B, Reactions and Synthesis”, F A Carey, R J Sundberg, Kluwer Academic/Plenum Publications, (2001 or later editions), “Organic Synthesis—The Disconnection Approach”, S Warren (Wiley), (1982 or later editions), “Designing Organic Syntheses” S Warren (Wiley) (1983 or later editions), “Guidebook To Organic Synthesis” R K Mackie and D M Smith (Longman) (1982 or later editions), etc., and the references therein as a guide.

The skilled chemist will exercise his judgement and skill as to the most efficient sequence of reactions for synthesis of a given target compound and will employ protecting groups as necessary. This will depend inter alia on factors such as the nature of other functional groups present in a particular substrate. Clearly, the type of chemistry involved will influence the choice of reagent that is used in the said synthetic steps, the need, and type, of protecting groups that are employed, and the sequence for accomplishing the protection/deprotection steps. These and other reaction parameters will be evident to the skilled person by reference to standard textbooks and to the examples provided herein.

Sensitive functional groups may need to be protected and deprotected during synthesis of a compound of the invention. This may be achieved by conventional methods, for example as described in “Protective Groups in Organic Synthesis” by TW Greene and PGM Wuts, John Wiley & Sons Inc (1999), and references therein.

Throughout this specification these abbreviations have the following meanings:

ACN—Acetonitrile CDI—Carbonyl diimidazole

BOC—tert-Butyl carbonate dba—Dibenzylideneacetone

DCM—Dichloromethane DMF—N,N-Dimethylformamide

DMSO—Dimethylsulfoxide EA—Ethyl acetate

IPA—iso-Propyl alcohol NMP—N-Methylpyrolidinone

PCC—Pyridinium chlorochromate PE—Petroleum ether

PMB—para-Methoxybenzyl TFA—Trifluoroacetic acid

THF—Tetrahydrofuran

Certain compounds of the invention can be made according to the following general schemes. Certain compounds of the invention can be made according to or analogously to the methods described in Examples 1 to 17.

Certain compounds of formula (I) can be made by Scheme A:—

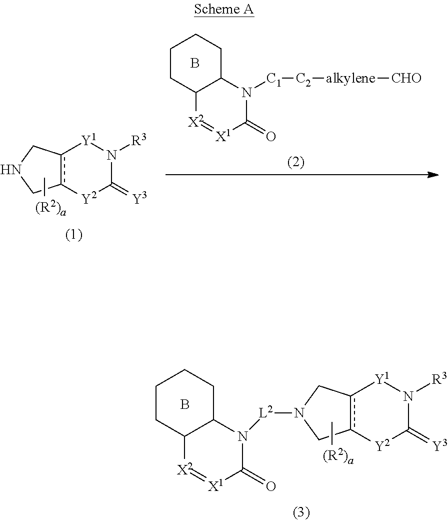

Amine (1) can be converted to (3) (a subset of compounds of formula (I)) via reductive amination with aldehyde (2). The reaction can be performed using a borohydride reagent, such as tetramethylammonium triacetoxyborohydride or sodium triacetoxyborohydride, in a solvent, such as THF or 1,2-dichloroethane, at a temperature from room temperature to 80° C. Addition of 4A sieves is optional.

Following Scheme A but using aldehyde (4), compound (5) (a subset of compounds of formula (I)) can be prepared.

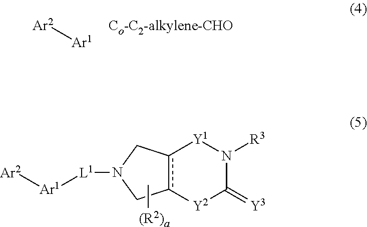

Following Scheme A but using amine (6), compound (7) (a subset of compounds of formula (II)) can be prepared.

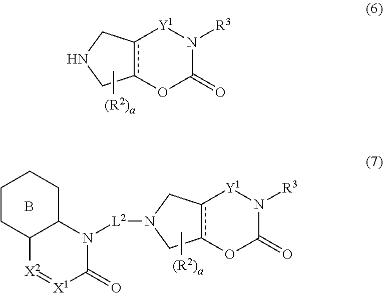

Following Scheme A but using amine (6) and aldehyde (4), compound (8) (a subset of compounds of formula (II)) can be prepared.

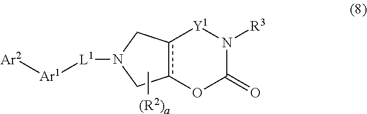

Following Scheme A but using amine (9) and aldehyde (4), compound (10) (a subset of compounds of formula (III)) can be prepared.

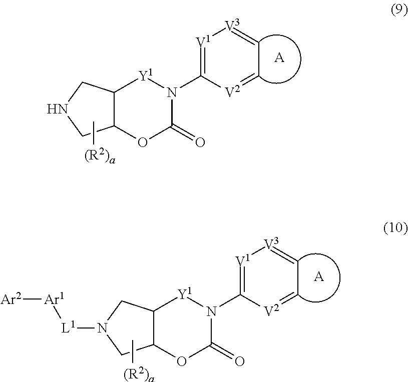

Following Scheme A but using amine (9) and aldehyde (2), compound (11) (a subset of compounds of formula (III)) can be prepared.

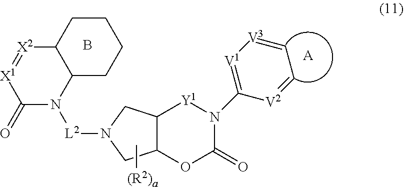

Following Scheme A, but using aldehyde (12) and amine (13), compound (14) (a subset of compounds of formula (IV)) can be prepared.

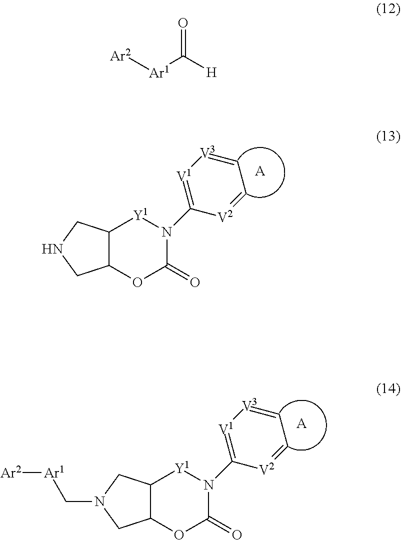

Following Scheme A, but using aldehyde (15) and amine (13), compound (16) (a subset of compounds of formula (V)) can be prepared.

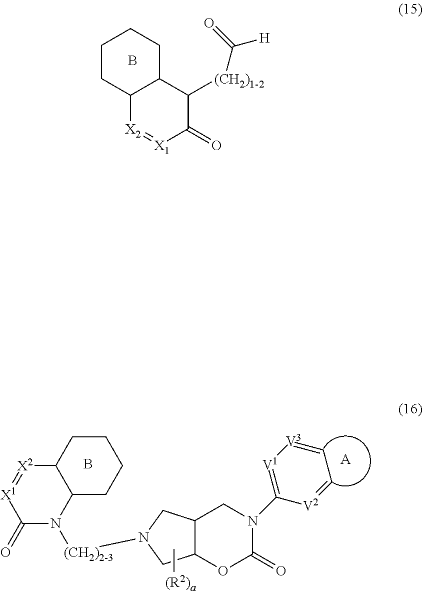

Aldehyde (15) can be made by Scheme B:—

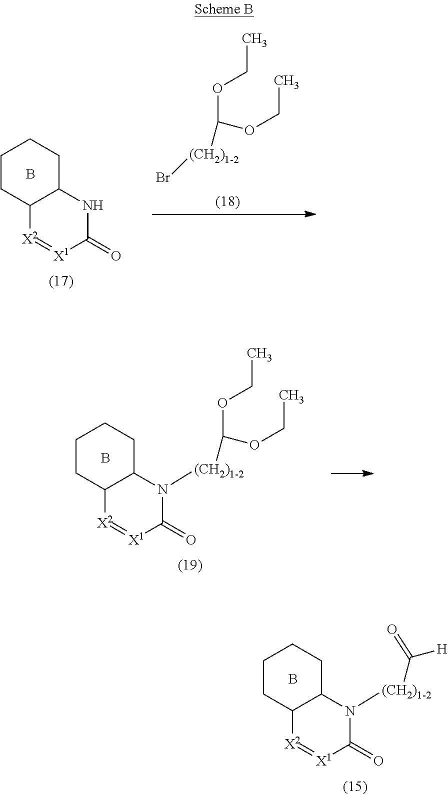

Reaction of pyridone (17) with commercially available bromo acetals (18) can generate pyridone acetals (19). The alkylation reaction can be carried out in the presence of a base, such as Cs₂CO₃, in a solvent, such as dry NMP, at a temperature from 50-100° C. Hydrolysis of pyridone acetals (19) to give the requisite aldehyde (15) can be effected using a strong acid, such as concentrated HCl, in a solvent, such as ACN, at room temperature.

Amine (13) can be made by Scheme C:—

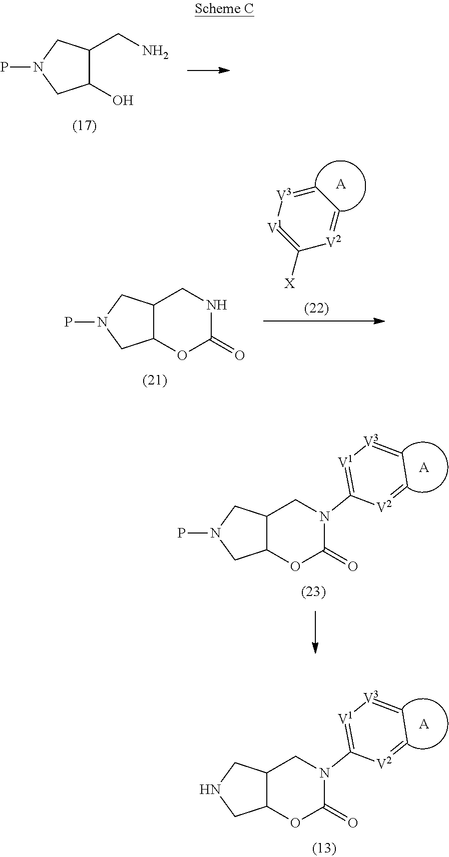

Reaction of protected amine (20), where P represents a standard nitrogen protecting group, such as BOC, with a carbonylating reagent, such as CDI, in a solvent, such as dioxane or ACN, at a temperature from 80-100° C. can generate (21). Addition of a base, such as K₂CO₃, is optional. Cross coupling reaction of (21), with (22), where X═Br or Cl, can generate protected amine (23). The cross coupling reaction can be copper catalysed, using for example CuI, in the presence of a diamine, such as 1,2-diaminocyclohexane or N,N-dimethyl-1,2-ethanediamine, in the presence of a base, such as K₂CO₃, in a solvent, such as dioxane or toluene, at a temperature from 70-110° C. The cross coupling reaction can be palladium catalysed, using for example, Pd₂(dba)₃ or Pd(OAc)₂ in the presence of a phosphine, such as Xantphos or P(t-Bu)₃ or X-Phos, in the presence of a base, such as t-BuONa or Cs₂CO₃, in a solvent, such as dioxane or toluene, at a temperature from 80-120° C. The nitrogen protecting group in (23) can be deprotected to give the free amine (13) under standard conditions. In the case of BOC, by the action of TFA in DCM at room temperature.

Following Scheme B, but using tricyclic pyridone (24), tricyclic aldehyde (25) can be prepared.

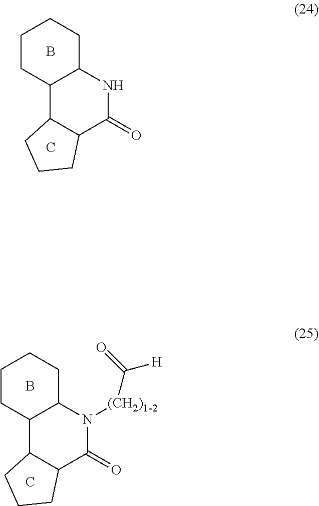

Following Scheme A, but using aldehyde (25) and amine (13), compound (26) (a subset of compounds of formula (VI)) can be prepared.

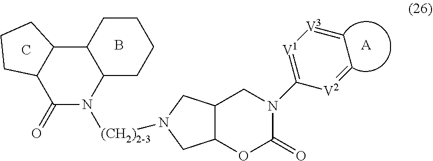

### Experimental

### Analytical Methods

NMR spectra were obtained on a LC Bruker AV400 using a 5 mm QNP probe (Method A), a Bruker AVIII 400 Nanobay using a 5 mm BBFQ with z-gradients (Method B), a Bruker AV1 Avance using a 5 mm QNP probe (Method C), a Bruker AV1 Avance using a 1H/13C Dual probe (Method D) or a Bruker ASCEND 400 MHz spectrometer (Method E).

MS was carried out on either a Waters ZQ MS (Method A and B), an Agilent Technologies 1200 series (Method C and D) using H₂O and ACN (0.1% formic acid—acidic pH; 0.1% ammonia—basic pH)—wavelengths were 254 and 210 nM, or a Shimadzu LCMS-2020 (Method E) using H₂O and MeOH (0.1% formic acid—acidic pH; 0.1% ammonia—basic pH)—wavelengths were 254 and 210 nM.

### Method A

Column: YMC-Triart C18, 5 μm, 50×2 mm. Flow rate: 0.8 mL/min. Injection volume 5 μL.

| Time (min) | H₂O % | ACN % |
|---|---|---|
| 0 | 95 | 0 |
| 4 | 0 | 95 |
| 4.4 | 0 | 95 |
| 4.5 | 95 | 5 |

| | |
|---|---|
| 4.5 | STOP |

### Method B

Column: YMC-Triart C18, 5 μm, 50×2 mm. Flow rate: 0.8 mL/min. Injection volume 5-10 μL

| Time (min) | H₂O (%) | ACN (%) |
|---|---|---|
| 0 | 95 | 0 |
| 2.0 | 95 | 0 |
| 12.0 | 0 | 95 |
| 14.0 | 0 | 95 |
| 14.2 | 95 | 0 |

### Method C

Column: Poroshell 120, 2.7 μm 50×2.1 mm. Flow rate: 0.8 mL/min. Injection volume 5-10 μL

| Time (min) | H₂O (%) | ACN (%) |
|---|---|---|
| 0.0 | 90 | 10 |
| 0.5 | 90 | 10 |
| 4.0 | 20 | 80 |
| 5.0 | 20 | 80 |
| 5.1 | 90 | 10 |
| 6.0 | 90 | 10 |

### Method D

Column: Poroshell 120, 2.7 μm 50×2.1 mm. Flow rate: 0.8 mL/min. Injection volume 5-10 μL

| Time (min) | H₂O (%) | ACN (%) |
|---|---|---|
| 0.0 | 90 | 10 |
| 0.5 | 90 | 10 |
| 5.0 | 20 | 80 |
| 6.0 | 20 | 80 |
| 6.1 | 90 | 10 |
| 7.0 | 90 | 10 |

### Method E

Column: Atlantis T3, 3 μm, 3.0×75 mm. Flow rate: 0.8 mL/min. Injection volume 1-10 μL

| Time (min) | H₂O (%) | MeOH (%) |
|---|---|---|
| 0.01 | 50 | 50 |
| 0.05 | 50 | 50 |
| 3.5 | 95 | 5 |
| 7.0 | 95 | 5 |
| 8.5 | 50 | 50 |
| 9.5 | 50 | 50 |

Preparative HPLC was performed using a Waters 3100 Mass detector (Method A) or Waters 2767 Sample Manager (Method B) using H₂O and ACN (0.1-% formic acid—acidic pH; 0.1% ammonia—basic pH).

### Method A

Column: XBridge™ prep C18, 5 μm OBD 19×100 mm. Flow rate: 20 mL/min.

### Method B

Column: XBridge™ prep C18, 5 μm OBD 19×100 mm. Flow rate: 20 mL/min.

### Example 1:—rel-1-{2-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]ethyl}-7-methoxy-1,2-dihydro-1,8-naphthyridin-2-one

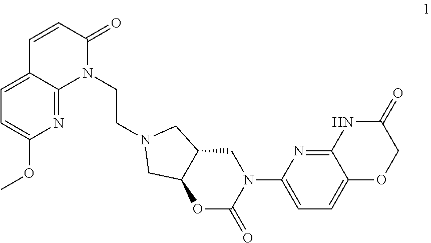 [SMILES: COc1ccc2ccc(=O)n(CCN3C[C@@H]4CN(c5ccc6c(n5)NC(=O)CO6)C(=O)O[C@H]4C3)c2n1]

### a) rel-tert-butyl (4aS*,7aR*)-2-oxo-octahydropyrrolo[3,4-e][1,3]oxazine-6-carboxylate 1a

To a solution of rel-tert-butyl (3S*,4R*)-3-(aminomethyl)-4-hydroxypyrrolidine-1-carboxylate (5.0 g, 23.1 mmol, WO 2006002047) in dioxane (100 mL) at 0° C. was added CDI (4.5 g, 27.8 mmol) and the mixture was heated at 100° C. for 17 h. The solvent was removed under reduced pressure and the residue was purified by silica gel chromatography (0-5% MeOH in DCM) to give a yellow solid of rel-tert-butyl (4aS*,7aR*)-2-oxo-octahydropyrrolo[3,4-e][1,3]oxazine-6-carboxylate 1a (1.2 g, 21%). TLC: R_{f}=0.4 (silica gel, DCM/MeOH=20/1, v/v). LC-MS (Method C) 243.1 [M+H]⁺; RT 2.90 min.

### b) rel-tert-butyl (4aS*,7aR*)-3-{4-[(4-methoxyphenyl)methyl]-3-oxo-2H,3H,4Hpyrido[3,2-b][1,4]oxazin-6-yl}-2-oxo-octahydropyrrolo[3,4-e][1,3]oxazine-6-carboxylate 1b

To a solution of rel-tert-butyl (4aS*,7aR*)-2-oxo-octahydropyrrolo[3,4-e][1,3]oxazine-6-carboxylate 1a (484 mg, 2.00 mmol) in toluene (20 mL) at room temperature was added 6-bromo-4-[(4-methoxyphenyl)methyl]-2H,3H,4H-pyrido[3,2-b][1,4]oxazin-3-one (prepared as described in WO2014108832) (696 mg, 2.00 mmol), (±)-trans-1,2-diaminocyclohexane (170 mg, 1.20 mmol), K₂CO3 (552 mg, 4.00 mmol) and CuI (228 mg, 1.20 mmol) and the mixture was heated at reflux for 17 h. The mixture was allowed to cool to room temperature, filtered and the filtrate was concentrated under reduced pressure. The residue was purified by silica gel chromatography using 20% EtOAc in petroleum ether then 5% MeOH in DCM to give a yellow solid of rel-tert-butyl (4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazine-6-carboxylate 1b (480 mg, 47%). TLC: R_{f}=0.4 (silica gel, DCM/MeOH=20:1, v/v). LC-MS (Method C) 511.2 [M+H]+; RT 4.25 min.

### c) rel-6-[(4aS*,7aR*)-2-oxo-octahydropyrrolo[3,4-e][1,3]oxazin-3-yl]-4-[(4-methoxyphenyl)methyl]-2H,3H,4H-pyrido[3,2-b][1,4]oxazin-3-one 1c

To a solution of rel-tert-butyl (4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazine-6-carboxylate 1b (1.14 g, 2.2 mmol) in DCM (10 mL) was added TFA (5 mL) and the mixture was stirred at room temperature for 1.5 h. The mixture was concentrated under reduced pressure and the residue was partitioned between EtOAc (50 mL) and saturated aqueous K₂CO3 (50 mL). The layers were separated and the aqueous layer was extracted with EtOAc (50 mL). The combined organic layers were washed with H₂O, brine, dried (Na₂SO₄), filtered and concentrated under reduced pressure. The residue was purified by silica gel chromatography using 2-5% MeOH in DCM to give a yellow solid of rel-6-[(4aS*,7aR*)-2-oxo-octahydropyrrolo[3,4-e][1,3]oxazin-3-yl]-4-[(4-methoxyphenyl)methyl]-2H,3H,4H-pyrido[3,2-b][1,4]oxazin-3-one 1c (180 mg, 20%). TLC: R_{f}=0.32 (silica gel, DCM/MeOH=10/1, v/v). LC-MS (Method C) 411.1 [M+H]+; RT 2.33 min. ¹H NMR (Method E) (CDCl₃): δ ppm 7.42 (d, J=8.4 Hz, 1H), 7.28-7.25 (m, 3H), 6.80 (d, J=8.4 Hz, 2H), 5.20 (s, 2H), 4.70 (s, 2H), 4.43 (m, 1H), 4.12 (dd, J=11.2, 5.6 Hz, 1H), 3.77 (m, 1H), 3.76 (s, 3H), 3.65 (m, 1H), 3.55-3.42 (m, 3H), 3.06 (t, J=9.6 Hz, 1H), 2.89 (t, J=10.8 Hz, 1H).

### d) rel-1-{2-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]ethyl}-7-methoxy-1,2-dihydro-1,8-naphthyridin-2-one 1d

To a solution of rel-6-[(4aS*,7aR*)-2-oxo-octahydropyrrolo[3,4-e][1,3]oxazin-3-yl]-4-[(4-methoxyphenyl)methyl]-2H,3H,4H-pyrido[3,2-b][1,4]oxazin-3-one 1c (230 mg, 0.56 mmol) and 2-(7-methoxy-2-oxo-1,8-naphthyridin-1-yl)acetaldehyde (prepared as described in WO2008009700) (122 mg, 0.56 mmol) in DCM (10 mL) was added AcOH (34 mg, 0.56 mmol) and the mixture was stirred at room temperature for 10 min. NaBH(OAc)₃ (594 mg, 2.80 mmol) was then added and stirring was continued at room temperature for 17 h. A saturated aqueous K₂CO₃ solution (50 mL) was added, the layers were separated and the aqueous layer was extracted with EtOAc (50 mL). The combined organic layers were washed with H₂O, brine, dried (Na₂SO₄), filtered and concentrated under reduced pressure.

The residue was purified by chromatography using 5% MeOH in DCM to give a yellow solid of rel-1-{2-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]ethyl}-7-methoxy-1,2-dihydro-1,8-naphthyridin-2-one 1d (320 mg, 93%). TLC: R_{f}=0.37 (silica gel, DCM/MeOH=20/1, v/v). LC-MS (Method C) 613.2 [M+H]⁺; RT 3.01 min.

### e) rel-1-{(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]ethyl}-7-methoxy-1,2-dihydro-1,8-naphthyridin-2-one 1

To a solution of rel-1-{2-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]ethyl}-7-methoxy-1,2-dihydro-1,8-naphthyridin-2-one 1d (320 mg, 0.52 mmol) in TFA (6 mL) at 00C was added CF₃SO₃H (0.6 mL) and the mixture was stirred at 0° C. for 1.5 h. The reaction was quenched with MeOH and the mixture was concentrated under reduced pressure. The residue was partitioned between EtOAc (50 mL) and saturated aqueous K₂CO3 (50 mL), the layers were separated and the aqueous layer was extracted with EtOAc (50 mL). The combined extracts were washed with H₂O, brine, dried (Na₂SO₄), filtered and concentrated under reduced pressure. The residue was purified by preparative TLC using 5% MeOH in DCM to give a yellow solid of rel-1-{2-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]ethyl}-7-methoxy-1,2-dihydro-1,8-naphthyridin-2-one 1 (74.1 mg, 35%). TLC: R_{f}=0.62 (silica gel, petroleum ether/EtOAc=10:1, v/v). LC-MS (Method E) 493 [M+H]⁺; RT 0.51 min. ¹H NMR (Method E) (CDCl₃): δ ppm 8.52 (br s, 1H), 7.73 (d, J=8.4 Hz, 1H), 7.55 (d, J=9.6 Hz, 1H), 7.39 (d, J=8.4 Hz, 1H), 7.29 (d, J=8.4 Hz, 1H), 6.64 (d, J=8.4 Hz, 1H), 6.53 (d, J=9.6 Hz, 1H), 4.64 (m, 2H), 4.62 (s, 2H), 4.47 (m, 1H), 4.23 (dd, J=5.6, 9.6 Hz, 1H), 4.05 (s, 3H), 3.64 (m, 1H), 3.39 (m, 1H), 3.32 (m, 1H), 3.15-3.20 (m, 1H), 3.02-3.10 (m, 2H), 2.86-2.91 (m, 1H), 2.54-2.60 (m, 1H).

### Example 2:—rel-(4aS*,7aR*)-6-(3-{7-methoxy-4-oxo-4H,5H-[1,2]oxazolo[3,4-c]quinolin-5-yl}propyl)-3-{3-oxo-2H,3H,4H-pyrido[3,2-b][1,4]oxazin-6-yl}-octahydropyrrolo[3,4-e][1,3]oxazin-2-one

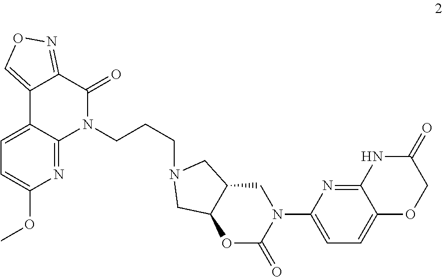 [SMILES: COc1ccc2c3conc3c(=O)n(CCCN3C[C@@H]4CN(c5ccc6c(n5)NC(=O)CO6)C(=O)O[C@H]4C3)c2n1]

### a) 5-(3,3-dimethoxypropyl)-7-methoxy-4H,5H-[1,2]oxazolo[3,4-c]quinolin-4-one 2a

A mixture of 7-methoxy-4H,5H-[1,2]oxazolo[3,4-c]quinolin-4-one (WO 2016024096 A1) (350 mg, 1.6 mmol), 3-bromo-1,1-dimethoxy-propane (385 mg, 2.1 mmol) and Cs₂CO3 (1.05 g, 3.2 mmol) in NMP (35 mL) was heated at 55° C. for 2 h. The mixture was diluted with H₂O and extracted with EtOAc (20 mL×3). The combined organic extracts were washed with H₂O, brine, dried over Na₂SO₄, filtered and concentrated under reduced pressure. The residue was purified by chromatography using petroleum ether/EtOAc, 8:1 to 3:1, v/v to give a white solid of 5-(3,3-dimethoxypropyl)-7-methoxy-4H,5H-[1,2]oxazolo[3,4-c]quinolin-4-one 2a (230 mg, 45%). TLC: R_{f}=0.59 (silica gel, petroleum ether/EtOAc=2:1, v/v).

### b) 3-{7-methoxy-4-oxo-4H,5H-[1,2]oxazolo[3,4-c]quinolin-5-yl}propanal 2b

To a solution of 5-(3,3-dimethoxypropyl)-7-methoxy-4H,5H-[1,2]oxazolo[3,4-c]quinolin-4-one 2a (220 mg, 0.69 mmol) in THF (30 mL) was added 1 M HCl (15 mL, 15 mmol) and the mixture was stirred at room temperature for 15 h. The mixture was adjusted to pH 8-9 with a saturated aqueous NaHCO₃ solution and extracted with EtOAc (20 mL×3). The combined organic extracts were washed with H₂O (30 mL), brine (30 mL), dried over Na₂SO₄, filtered and concentrated under reduced pressure to give a light yellow solid of 3-{7-methoxy-4-oxo-4H,5H-[1,2]oxazolo[3,4-c]quinolin-5-yl}propanal 2b (150 mg, 80%). TLC: R_{f}=0.38 (silica gel, EtOAc/petroleum ether=1:1, v/v). LC-MS (Method D) 273.0 [M+H]⁺; RT 3.15 min.

### c) rel-(4aS*,7aR*)-6-(3-{7-methoxy-4-oxo-4H,5H-[1,2]oxazolo[3,4-c]quinolin-5-yl}propyl)-3-{4-[(4-methoxyphenyl)methyl]-3-oxo-2H,3H,4H-pyrido[3,2-b][1,4]oxazin-6-yl}-octahydropyrrolo[3,4-e][1,3]oxazin-2-one 2c

To a solution of 3-{7-methoxy-4-oxo-4H,5H-[1,2]oxazolo[3,4-c]quinolin-5-yl}propanal 2b (66 mg, 0.24 mmol) and rel-6-[(4aS*,7aR*)-2-oxo-octahydropyrrolo[3,4-e][1,3]oxazin-3-yl]-4-[(4-methoxyphenyl)methyl]-2H,3H,4H-pyrido[3,2-b][1,4]oxazin-3-one 1c (99 mg, 0.24 mmol) in DCM (30 mL) was added NaBH(OAc)₃ (488 mg, 2.3 mmol) and the mixture was stirred at room temperature for 3 h. The mixture was diluted with a saturated aqueous NaHCO₃ solution and extracted with EtOAc (20 mL×3). The combined organic extracts were washed with H₂O, brine, dried over Na₂SO₄, filtered and concentrated under reduced pressure. The residue was purified by silica gel chromatography using DCM/MeOH, 40:1 to 20:1, v/v to give a light yellow solid of rel-(4aS*,7aR*)-6-(3-{7-methoxy-4-oxo-4H,5H-[1,2]oxazolo[3,4-c]quinolin-5-yl}propyl)-3-{4-[(4-methoxyphenyl)methyl]-3-oxo-2H,3H,4H-pyrido[3,2-b][1,4]oxazin-6-yl}-octahydropyrrolo[3,4-e][1,3]oxazin-2-one 2c (90 mg, 57%) TLC: R_{f}=0.43 (silica gel, DCM/MeOH=15:1, v/v).

### d) rel-(4aS*,7aR*)-6-(3-{7-methoxy-4-oxo-4H,5H-[1,2]oxazolo[3,4-c]quinolin-5-yl}propyl)-3-{3-oxo-2H,3H,4H-pyrido[3,2-b][1,4]oxazin-6-yl}-octahydropyrrolo[3,4-e][1,3]oxazin-2-one 2

To a solution of rel-(4aS*,7aR*)-6-(3-{7-methoxy-4-oxo-4H,5H-[1,2]oxazolo[3,4-c]quinolin-5-yl}propyl)-3-{4-[(4-methoxyphenyl)methyl]-3-oxo-2H,3H,4H-pyrido[3,2-b][1,4]oxazin-6-yl}-octahydropyrrolo[3,4-e][1,3]oxazin-2-one 2c (260 mg, 0.39 mmol) in DCM (30 mL) at 00C was added TFA (2.0 mL) and CF₃SO₃H (0.5 mL) and the mixture was allowed to warm to room temperature and stirred for 1 h. The mixture was adjusted to pH 8-9 with a saturated aqueous K₂CO₃ solution (40 mL) and then extracted with EtOAc (30 mL×3). The combined organic extracts were washed with H₂O, brine, dried over Na₂SO₄, filtered and concentrated under reduced pressure The residue was purified by preparative TLC using DCM/MeOH, 10:1, v/v to give a light yellow solid of rel-(4aS*,7aR*)-6-(3-{7-methoxy-4-oxo-4H,5H-[1,2]oxazolo[3,4-c]quinolin-5-yl}propyl)-3-{3-oxo-2H,3H,4H-pyrido[3,2-b][1,4]oxazin-6-yl}-octahydropyrrolo[3,4-e][1,3]oxazin-2-one 2 (40 mg, 54%). TLC: R_{f}=0.32 (silica gel, DCM/MeOH=10:1, v/v). LC-MS (Method D) 547.2 [M+H]⁺; RT 2.63 min. ¹H NMR (Method E) (DMSO-d6): 5 ppm 11.2 (br s, 1H), 9.95 (s, 1H), 8.00 (d, J=8.8 Hz, 1H), 7.41 (d, J=8.4 Hz, 1H), 7.16 (d, J=8.4 Hz, 1H), 7.10 (d, J=2.0 Hz, 1H), 7.01 (dd, J=8.8, 2.0 Hz, 1H), 4.65 (s, 2H), 4.51 (m, 1H), 4.32 (m, 2H), 4.03 (m, 1H), 3.89 (s, 3H), 3.53 (m, 1H), 3.05 (m, 2H), 2.86-2.72 (m, 3H), 2.57 (m, 1H), 2.42 (m, 1H), 1.81 (m, 2H).

### Example 3:—rel-6-(3-{[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]methyl}-2-hydroxyphenyl)pyridine-2-carbonitrile

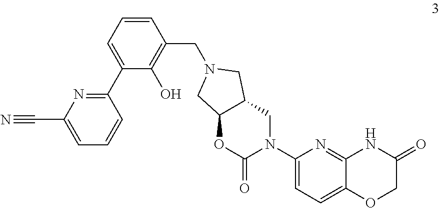 [SMILES: N#Cc1cccc(-c2cccc(CN3C[C@@H]4CN(c5ccc6c(n5)NC(=O)CO6)C(=O)O[C@H]4C3)c2O)n1]

### a) rel-(4aS*,7aR*)-3-{3-oxo-2H,3H,4H-pyrido[3,2-b][1,4]oxazin-6-yl}-octahydropyrrolo[3,4-e][1,3]oxazin-2-one 3a

To a solution of rel-tert-butyl (4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazine-6-carboxylate 1b (190 mg, 0.37 mmol) in DCM (3 mL) was added TFA (3 mL) and the mixture was stirred at room temperature for 1 h. CF₃SO₃H (1 mL) was added and stirring was continued at room temperature for 1 h. The solvent was removed under reduced pressure and the residue was re-dissolved in DCM (30 mL), neutralized with TEA and concentrated. The residue was purified by silica gel chromatography using 10% MeOH in DCM to give a solid of rel-(4aS*,7aR*)-3-{3-oxo-2H,3H,4H-pyrido[3,2-b][1,4]oxazin-6-yl}-octahydropyrrolo[3,4-e][1,3]oxazin-2-one 3a (80 mg, 75%). TLC: R_{f}=0.36 (silica gel, DCM/MeOH=8/1, v/v).

### b) rel-6-(3-{[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]methyl}-2-hydroxyphenyl)pyridine-2-carbonitrile 3

A mixture of 6-(3-formyl-2-hydroxyphenyl)pyridine-2-carbonitrile (prepared as described in WO2014170821) (62 mg, 0.28 mmol) and rel-(4aS*,7aR*)-3-{3-oxo-2H,3H,4H-pyrido[3,2-b][1,4]oxazin-6-yl}-octahydropyrrolo[3,4-e][1,3]oxazin-2-one 3a (75 mg, 0.26 mmol) in DCM (20 mL) was stirred at room temperature for 30 min. NaBH(OAc)₃ (164 mg, 0.77 mmol) was then added and stirring was continued at room temperature for 17 h. The mixture was diluted with DCM (30 mL) and washed with H₂O, brine and concentrated under reduced pressure. The residue was purified by preparative TLC (DCM/MeOH, 20:1, v/v) to give a white solid of rel-6-(3-{[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]methyl}-2-hydroxyphenyl)pyridine-2-carbonitrile (80 mg, 62%). TLC: R_{f}=0.35 (silica gel, DCM/MeOH=10:1, v/v). LC-MS (Method C) 499.2 [M+H]⁺; RT 2.44 min. ¹H NMR (Method E) (CDCl₃): δ ppm 8.28 (br s, 1H), 8.19 (d, J=8.3 Hz, 1H), 7.92 (t, J=8.0 Hz, 1H), 7.78 (d, J=7.98, 1H), 7.62 (d, J=7.5 Hz, 1H), 7.30 (m, 1H), 7.29-7.22 (m, 2H), 6.95 (t, J=7.7 Hz, 1H), 4.64 (s, 2H), 4.52 (m, 1H), 4.12 (m, 1H), 4.05 (m, 2H), 3.59 (t, J=11.5 Hz, 1H), 3.34-3.23 (m, 2H), 3.07 (t, J=9.4 Hz, 1H), 2.79 (s, J=10.3 Hz, 1H), 2.59 (m, 1H).

### Example 4:—rel-1-{3-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]propyl}-1,2-dihydroquinolin-2-one

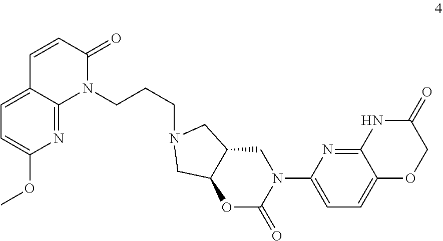 [SMILES: COc1ccc2ccc(=O)n(CCCN3C[C@@H]4CN(c5ccc6c(n5)NC(=O)CO6)C(=O)O[C@H]4C3)c2n1]

### a) rel-1-{3-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]propyl}-1,2-dihydroquinolin-2-one 4a

To a solution of rel-6-[(4aS*,7aR*)-2-oxo-octahydropyrrolo[3,4-e][1,3]oxazin-3-yl]-4-[(4-methoxyphenyl)methyl]-2H,3H,4H-pyrido[3,2-b][1,4]oxazin-3-one 1c (110 mg, 0.26 mmol) and 3-(2-oxo-1,2-dihydroquinolin-1-yl)propanal (54 mg, 0.26 mmol) in DCM (10 mL) was added AcOH (17 mg, 0.26 mmol) and the mixture was stirred at room temperature for 10 min. NaBH(OAc)₃ (322 mg, 1.3 mmol) was then added and stirring was continued at room temperature for 17 h. A saturated aqueous K₂CO₃ solution (50 mL) was added, the layers were separated and the aqueous phase was extracted with EtOAc (50 mL). The combined organic layers were washed with H₂O, brine, dried (Na₂SO₄), filtered and concentrated under reduced pressure to give rel-1-{3-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]propyl}-1,2-dihydroquinolin-2-one 4a (150 mg, 94%). TLC: R_{f}=0.37 (silica gel, DCM/MeOH=20/1, v/v). ¹H NMR (Method E) (CDCl₃): δ ppm 7.69 (d, J=9.2 Hz, 1H), 7.56-7.60 (m, 2H), 7.42-7.47 (m, 2H), 7.23-7.29 (m, 2H), 6.81 (d, J=8.4 Hz, 2H), 6.71 (d, J=9.6 Hz, 1H), 5.21 (s, 2H), 4.69 (s, 2H), 4.35-4.48 (m, 3H), 4.10 (dd, J=11.2, 5.6 Hz, 1H), 3.77 (s, 3H), 3.49 (t, J=11.6 Hz, 1H), 3.15-3.22 (m, 2H), 3.01 (t, J=9.2 Hz, 1H), 2.80-2.88 (m, 2H), 2.72 (t, J=9.6 Hz, 1H), 2.52-2.56 (m, 1H), 1.97 (m, 2H).

### b) rel-1-{3-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]propyl}-1,2-dihydroquinolin-2-one 4

To a solution of rel-1-{3-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]propyl}-1,2-dihydroquinolin-2-one 4a (150 mg, 0.25 mmol) in TFA (3 mL) at 0° C. was added CF₃SO₃H (0.2 mL) and the mixture was stirred at 0° C. for 1.5 h. The reaction was quenched with MeOH and the mixture was concentrated under reduced pressure. The residue was partitioned between EtOAc (50 mL) and saturated aqueous K₂CO₃ (50 mL), the layers were separated and the aqueous layer was extracted with EtOAc (50 mL). The combined organic layers were washed with H₂O, brine, dried (Na₂SO₄), filtered and concentrated under reduced pressure. The residue was purified by preparative TLC using 5% MeOH in DCM to give a yellow solid of rel-1-{3-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]propyl}-1,2-dihydroquinolin-2-one 4 (15 mg, 13%). TLC: R_{f}=0.3 (silica gel, DCM/MeOH=20/1, v/v). LC-MS (Method E) 476 [M+H]⁺; RT 0.50 min. ¹H NMR (Method E) (CDCl₃): δ ppm 8.64 (br s, 1H), 7.68 (d, J=9.2 Hz, 1H), 7.56 (m, 2H), 7.44 (d, J=8.8 Hz, 1H), 7.34 (d, J=8.4 Hz, 1H), 7.28 (d, J=8.8 Hz, 1H), 7.22 (d, J=7.6 Hz, 1H), 6.69 (d, J=9.6 Hz, 1H), 4.65 (s, 2H), 4.47 (m, 1H), 4.37 (m, 2H), 4.15 (dd, J=10.8, 5.2 Hz, 1H), 3.60 (t, J=11.6 Hz, 1H), 3.20 (m, 2H), 3.02 (m, 1H), 2.83 (m, 2H), 2.74 (m, 1H), 2.55 (m, 1H), 1.93 (m, 2H).

### Example 5:—rel-1-{2-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]ethyl}-7-(2-hydroxyethoxy)-1,2-dihydro-1,8-naphthyridin-2-one

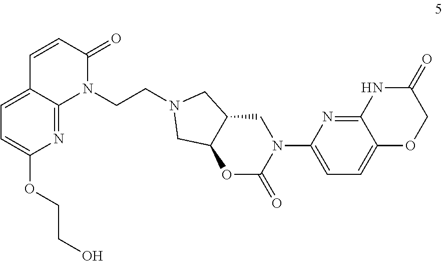 [SMILES: O=C1COc2ccc(N3C[C@H]4CN(CCn5c(=O)ccc6ccc(OCCO)nc65)C[C@@H]4OC3=O)nc2N1]

### a) 7-chloro-1-(2,2-diethoxyethyl)-1,2-dihydro-1,8-naphthyridin-2-one 5a

To a solution of 7-chloro-1,2-dihydro-1,8-naphthyridin-2-one (1.80 g, 10.0 mmol) and 2-bromo-1,1-diethoxyethane (4.92 g, 25.0 mmol) in DMF (25 mL) was added Cs₂CO₃ (4.90 g, 15.0 mmol) and the mixture heated at 70° C. under N₂ overnight. The mixture was diluted with H₂O (200 mL), extracted with EtOAc (100 mL×3) and the combined organic extracts were washed with H₂O (200 mL×2), brine (100 mL) and concentrated under reduced pressure. The residue was purified by chromatography (EtOAc/petroleum ether, 1:5 to 1:2, v/v) to afford a white solid of 7-chloro-1-(2,2-diethoxyethyl)-1,2-dihydro-1,8-naphthyridin-2-one 5a (1.80 g, 61%). TLC: R_{f}=0.45 (silica gel, petroleum ether/EtOAc=2:1, v/v). ¹H NMR (Method E) (CDCl₃): δ ppm 7.78 (d, J=8.0 Hz, 1H), 7.61 (d, J=9.6 Hz, 1H), 7.15 (d, J=8.0 Hz, 1H), 6.72 (d, J=9.6 Hz, 1H), 5.10 (t, J=5.6 Hz, 1H), 4.67 (d, J=5.6 Hz, 2H), 3.79 (m, 2H), 3.54 (m, 2H), 1.11 (t, J=7.2 Hz, 6H).

### b) 1-(2,2-diethoxyethyl)-7-(2-hydroxyethoxy)-1,2-dihydro-1,8-naphthyridin-2-one 5b

7-chloro-1-(2,2-diethoxyethyl)-1,2-dihydro-1,8-naphthyridin-2-one 5a (592 mg, 2 mmol) and Cs₂CO₃ (978 mg, 3 mmol) were suspended in ethylene glycol (5 mL) and the mixture was heated at 100° C. in the microwave for 1 h. The mixture was diluted with H₂O (30 mL), extracted with EtOAc (30 mL×3) and the combined organic extracts were washed with H₂O (50 mL×2), brine (50 mL), dried over Na₂SO₄, filtered and concentrated under reduced pressure to give a yellow solid of 1-(2,2-diethoxyethyl)-7-(2-hydroxyethoxy)-1,2-dihydro-1,8-naphthyridin-2-one 5b (574 mg, 89%) which was used without further purification. TLC: R_{f}=0.33 (silica gel, petroleum ether/EtOAc=1:1, v/v). LC-MS (Method C) 277.1 [M-OEt]⁺, RT 2.76 min.

### c) 2-[7-(2-hydroxyethoxy)-2-oxo-1,2-dihydro-1,8-naphthyridin-1-yl]acetaldehyde 5c

To a solution of 1-(2,2-diethoxyethyl)-7-(2-hydroxyethoxy)-1,2-dihydro-1,8-naphthyridin-2-one 5b (574 mg, 1.8 mmol) in dioxane (6 mL) was added 3 M HCl (6 mL, 18 mmol) and the mixture was stirred at room temperature for 2 h. The mixture was adjusted to pH 8-9 with a saturated aqueous NaHCO₃ solution and extracted with EtOAc (20 mL×3). The combined organic extracts were washed with H₂O (30 mL), brine (30 mL), dried over Na₂SO₄, filtered and concentrated under reduced pressure. The residue was purified by chromatography using petroleum ether/EtOAc, 5:1 to 1:1, v/v to give a pale yellow oil of 2-[7-(2-hydroxyethoxy)-2-oxo-1,2-dihydro-1,8-naphthyridin-1-yl]acetaldehyde 5c (240 mg). TLC: R_{f}=0.20 (silica gel, petroleum ether/EtOAc=1:1, v/v). LC-MS (Method D) 249.1 [M+H]⁺, RT 2.17 min.

### d) rel-1-{2-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]ethyl}-7-(2-hydroxyethoxy)-1,2-dihydro-1,8-naphthyridin-2-one 5d

A mixture of 2-[7-(2-hydroxyethoxy)-2-oxo-1,2-dihydro-1,8-naphthyridin-1-yl]acetaldehyde 5c (50 mg, 0.20 mmol), rel-6-[(4aS*,7aR*)-2-oxo-octahydropyrrolo[3,4-e][1,3]oxazin-3-yl]-4-[(4-methoxyphenyl)methyl]-2H,3H,4H-pyrido[3,2-b][1,4]oxazin-3-one 1c (85 mg, 0.20 mmol) and AcOH (24 mg, 0.40 mmol) in 1,2-dichloroethane (4 mL) was stirred at room temperature for 10 min. NaBH(OAc)₃ (212 mg, 1.0 mmol) was then added and stirring was continued at room temperature for 17 h. The mixture was diluted with a saturated aqueous NaHCO₃ solution and extracted with EtOAc (20 mL×3). The combined organic extracts were washed with H₂O, brine, dried over Na₂SO₄, filtered and concentrated under reduced pressure to give a yellow solid of rel-1-{2-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]ethyl}-7-(2-hydroxyethoxy)-1,2-dihydro-1,8-naphthyridin-2-one 5d (128 mg, 100%). TLC R_{f}=0.44 (silica gel, DCM/MeOH=15:1, v/v). LC-MS (Method C) 643.3 [M+H]⁺; RT 2.69 min.

### e) rel-1-{2-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]ethyl}-7-(2-hydroxyethoxy)-1,2-dihydro-1,8-naphthyridin-2-one 5

To a solution of rel-1-{2-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]ethyl}-7-(2-hydroxyethoxy)-1,2-dihydro-1,8-naphthyridin-2-one 5d (128 mg, 0.20 mmol) in TFA (3.0 mL) at 0° C. was added CF₃SO₃H (0.3 mL) and the mixture was allowed to warm to room temperature and stirred for 1 h. The mixture was adjusted to pH 8-9 with a saturated aqueous K₂CO₃ solution (40 mL) and then extracted with EtOAc (30 mL×3). The combined organic extracts were washed with H₂O, brine, dried over Na₂SO₄, filtered and concentrated under reduced pressure The residue was purified by preparative TLC (DCM/MeOH, 10:1, v/v) then preparative HPLC to give a light yellow solid of rel-1-{2-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]ethyl}-7-(2-hydroxyethoxy)-1,2-dihydro-1,8-naphthyridin-2-one 5 (50 mg, 48%). TLC: R_{f}=0.50 (silica gel, DCM/MeOH=10:1, v/v). LC-MS (Method D) 523.2 [M+H]⁺; RT 1.99 min. ¹H NMR (Method E) (DMSO-d6): 5 ppm 8.06 (d, J=8.4 Hz, 1H), 7.86 (d, J=9.2 Hz, 1H), 7.27 (d, J=8.4 Hz, 1H), 7.00 (d, J=8.0 Hz, 1H), 6.73 (d, J=8.4 Hz, 1H), 6.50 (d, J=9.6 Hz, 1H), 4.52 (m, 3H), 4.43 (m, 4H), 4.01 (m, 1H), 3.78 (m, 2H), 3.53 (m, 1H), 3.16 (m, 2H), 2.99-2.82 (m 3H), 2.67 (m, 1H), 2.42 (m, 1H).

### Example 6:—rel-1-{3-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]propyl}-7-(3-aminopropoxy)-1,2-dihydro-1,8-naphthyridin-2-one

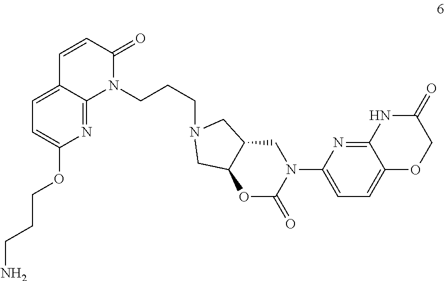 [SMILES: NCCCOc1ccc2ccc(=O)n(CCCN3C[C@@H]4CN(c5ccc6c(n5)NC(=O)CO6)C(=O)O[C@H]4C3)c2n1]

### a) 7-chloro-1-(3,3-dimethoxypropyl)-1,2-dihydro-1,8-naphthyridin-2-one 6a

A mixture of 7-chloro-1,2-dihydro-1,8-naphthyridin-2-one (1 g, 5.53 mmol), 3-bromo-1,1-dimethoxypropane (1.1 g, 6.09 mmol) and K₂CO₃ (1.1 g, 8.31 mmol) in DMF (20 mL) was heated at 70° C. for 4 h. The mixture was then allowed to cool to room temperature, poured into H₂O (30 mL) and extracted with EtOAc (30 mL×3). The combined organic extracts were washed with H₂O (50 mL×2), brine (50 mL), dried over Na₂SO₄, filtered and concentrated under reduced pressure. The residue was purified by silica gel chromatography using 30% EtOAc/petroleum ether to give a yellow solid of 7-chloro-1-(3,3-dimethoxypropyl)-1,2-dihydro-1,8-naphthyridin-2-one 6a (1 g, 64%). TLC: R_{f}=0.54 (silica gel, EtOAc/petroleum ether=1:1, v/v).

### b) tert-butyl N-(3-{[8-(3,3-dimethoxypropyl)-7-oxo-7,8-dihydro-1,8-naphthyridin-2-yl]oxy}propyl)carbamate 6b

A mixture of 7-chloro-1-(3,3-dimethoxypropyl)-1,2-dihydro-1,8-naphthyridin-2-one 6a (200 mg, 0.71 mmol), tert-butyl N-(3-hydroxypropyl)carbamate (310 mg, 1.77 mmol) and t-BuOK (238 mg, 2.12 mmol) in DMF (20 mL) was stirred at room temperature for 17 h. The mixture was diluted with H₂O (30 mL), extracted with EtOAc (30 mL×3) and the combined organic extracts were washed with H₂O (50 mL×2), brine (50 mL), dried over Na₂SO₄, filtered and concentrated under reduced pressure. The residue was purified by silica gel chromatography using petroleum ether/EtOAc, 2:1, v/v to EtOAc/DCM, 1:1, v/v to give a yellow solid of tert-butyl N-(3-{[8-(3,3-dimethoxypropyl)-7-oxo-7,8-dihydro-1,8-naphthyridin-2-yl]oxy}propyl)carbamate 6b (80 mg, 27%). TLC: R_{f}=0.49 (silica gel, DCM/EtOAc=1:1, v/v). LC-MS (Method D) 444.2 [M+H]⁺; RT 4.39 min.

### c) tert-butyl N-(3-{[7-oxo-8-(3-oxopropyl)-7,8-dihydro-1,8-naphthyridin-2yl]oxy}propyl)carbamate 6c

To a solution of tert-butyl N-(3-{[8-(3,3-dimethoxypropyl)-7-oxo-7,8-dihydro-1,8-naphthyridin-2-yl]oxy}propyl)carbamate 6b (80 mg, 0.19 mmol) in THF (10 mL) was added 1 M HCl (1 mL, 1 mmol) and the mixture was stirred at room temperature for 16 h. The mixture was adjusted to pH 8-9 with a saturated aqueous NaHCO₃ solution and extracted with EtOAc (20 mL×3). The combined organic extracts were washed with H₂O (30 mL), brine (30 mL), dried over Na₂SO₄, filtered and concentrated under reduced pressure to give tert-butyl N-(3-{[7-oxo-8-(3-oxopropyl)-7,8-dihydro-1,8-naphthyridin-2yl]oxy}propyl)carbamate 6c (65 mg, 91%). TLC: R_{f}=0.4 (silica gel, petroleum ether/EtOAc=1:1, v/v).

### d) rel-tert-butyl N-{3-[(8-{3-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]propyl}-7-oxo-7,8-dihydro-1,8-naphthyridin-2-yl)oxy]propyl}carbamate 6d

To a solution of tert-butyl N-(3-{[7-oxo-8-(3-oxopropyl)-7,8-dihydro-1,8-naphthyridin-2yl]oxy}propyl)carbamate 6c (70 mg, 0.19 mmol) and rel-6-[-(4aS*,7aR*)-2-oxo-octahydropyrrolo[3,4-e][1,3]oxazin-3-yl]-4-[(4-methoxyphenyl)methyl]-2H,3H,4H-pyrido[3,2-b][1,4]oxazin-3-one 1c (72 mg, 0.18 mmol) in DCM (10 mL) was added NaBH(OAc)₃ (488 mg, 2.3 mmol) and the mixture was stirred at room temperature for 4 h. The mixture was diluted with a saturated aqueous NaHCO₃ solution and extracted with EtOAc (20 mL×3). The combined organic extracts were washed with H₂O, brine, dried over Na₂SO₄, filtered and concentrated under reduced pressure. The residue was purified by silica gel chromatography using DCM/MeOH, 40:1 to 20:1, v/v to give a white solid of rel-tert-butyl N-{3-[(8-{3-[-(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]propyl}-7-oxo-7,8-dihydro-1,8-naphthyridin-2-yl)oxy]propyl}carbamate 6d (90 mg, 63%). TLC: R_{f}=0.4 (silica gel, DCM/MeOH=15:1, v/v). LC-MS (Method D) 770.3 [M+H]⁺; RT 4.39 min.

### (e) rel-1-{3-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]propyl}-7-(3-aminopropoxy)-1,2-dihydro-1,8-naphthyridin-2-one 6

To a solution of rel-tert-butyl N-{3-[(8-{3-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]propyl}-7-oxo-7,8-dihydro-1,8-naphthyridin-2-yl)oxy]propyl}carbamate 6d (90 mg, 0.12 mmol) in DCM (25 mL) at 0° C. was added TFA (2.0 mL) and CF₃SO₃H (0.4 mL) and the mixture was allowed to warm to room temperature and stirred for 1 h. The mixture was adjusted to pH 8-9 with a saturated aqueous K₂CO₃ solution (40 mL) and then extracted with EtOAc (30 mL×3). The combined organic extracts were washed with H₂O, brine, dried over Na₂SO₄, filtered and concentrated under reduced pressure The residue was purified by preparative TLC (DCM/MeOH, 10:1, v/v) to give a white solid of rel-1-{3-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]propyl}-7-(3-aminopropoxy)-1,2-dihydro-1,8-naphthyridin-2-one 6 (30 mg, 47%). TLC: R_{f}=0.14 (silica gel, DCM/MeOH=6:1, v/v). LC-MS (Method D) 550.2 [M+H]⁺; RT 1.65 min. ¹H NMR (Method E) (DMSO-d6): δ ppm 8.07 (d, J=8.4 Hz, 1H), 7.87 (d, J=9.2 Hz, 1H), 7.40 (d, J=8.4 Hz, 1H), 7.15 (d, J=8.4 Hz, 1H), 6.74 (d, J=8.4 Hz, 1H), 6.50 (d, J=9.2 Hz, 1H), 4.65 (s, 2H), 4.47 (m, 3H), 4.40 (m, 2H), 4.02 (m, 1H), 3.02 (m, 2H), 2.92 (m, 2H), 2.79-2.61 (m, 3H), 2.54 (m, 1H), 2.38 (m, 2H), 2.05 (m, 2H), 1.82 (m, 2H).

### Example 7:—rel-1-{2-[(4aS*,7aR*-2-oxo-3-{3-oxo-2H,3H,4H-pyrido[3,2-b][1,4]oxazin-6-yl}-octahydropyrrolo[3,4-e][1,3]oxazin-6-yl]ethyl}-7-(pyridin-3-yloxy)-1,2-dihydro-1,8-naphthyridin-2-one

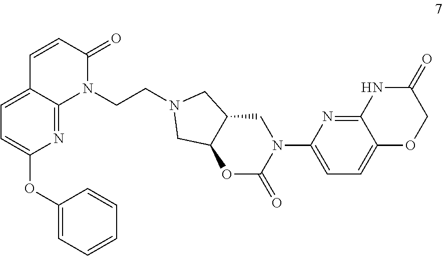 [SMILES: O=C1COc2ccc(N3C[C@H]4CN(CCn5c(=O)ccc6ccc(Oc7ccccc7)nc65)C[C@@H]4OC3=O)nc2N1]

### a) 1-(2,2-diethoxyethyl)-7-(pyridin-3-yloxy)-1,2-dihydro-1,8-naphthyridin-2-one 7a

To a solution of 7-chloro-1-(2,2-diethoxyethyl)-1,2-dihydro-1,8-naphthyridin-2-one 5a (360 mg, 1.22 mmol) and pyridin-3-ol (174 mg, 1.83 mmol) in DMF (10 mL) was added Cs₂CO₃ (800 mg, 2.45 mmol) and the mixture was heated to 70° C. under a nitrogen atmosphere for 17 h. The mixture was diluted with water (100 mL), extracted with EtOAc (3×30 mL) and the combined organics extracts were washed with water (2×100 mL), brine (50 mL) and concentrated under reduced pressure to give a colourless oil of 1-(2,2-diethoxyethyl)-7-(pyridin-3-yloxy)-1,2-dihydro-1,8-naphthyridin-2-one 7a (430 mg, 100%). TLC: R_{f}=0.30 (silica gel, petroleum ether/EtOAc, 2:1, v/v). LC-MS (Method C) 310.10 [M-OEt]*; RT 3.29 min.

### b) 2-[2-oxo-7-(pyridin-3-yloxy)-1,2-dihydro-1,8-naphthyridin-1-yl]acetaldehyde 7b

To a solution of 1-(2,2-diethoxyethyl)-7-(pyridin-3-yloxy)-1,2-dihydro-1,8-naphthyridin-2-one 7a (430 mg, 1.22 mmol) in THF (20 mL) was added aqueous HCl (2.0 M, 12.2 mL, 24.4 mmol) and the mixture was stirred at room temperature for 2 h. The mixture was adjusted to pH 8-9 with saturated aqueous NaHCO₃ and extracted with EtOAc (20 mL×3). The combined organic extracts were washed with brine (50 mL), dried over Na₂SO₄, filtered and concentrated under reduced pressure. The residue was purified by chromatography (petroleum ether/EtOAc, 5:1 to 1:1, v/v) to give a waxy solid of 2-[2-oxo-7-(pyridin-3-yloxy)-1,2-dihydro-1,8-naphthyridin-1-yl]acetaldehyde 7b (240 mg, 70%). TLC: R_{f}=0.14 (silica gel, petroleum ether/EtOAc=2:1, v/v). LC-MS (Method C) 282.1 [M+H]⁺; RT 2.46 min. ¹H NMR (Method E) (CDCl₃): 5 ppm 9.40 (s, 1H), 8.54 (br s, 2H), 7.92 (d, J=8.4 Hz, 1H), 7.68 (d, J=9.5 Hz, 1H), 7.44 (d, J=8.0 Hz, 1H), 7.40 (br s, 1H), 6.89 (d, J=8.4 Hz, 1H), 6.66 (d, J=9.5 Hz, 1H), 4.94 (s, 2H).

### c) rel-1-{2-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]ethyl}-7-(pyridin-3-yloxy)-1,2-dihydro-1,8-naphthyridin-2-one 7c

A mixture of 2-[2-oxo-7-(pyridin-3-yloxy)-1,2-dihydro-1,8-naphthyridin-1-yl]acetaldehyde 7b (66 mg, 0.23 mmol, 1 eq) and rel-6-[(4aS*,7aR*)-2-oxo-octahydropyrrolo[3,4-e][1,3]oxazin-3-yl]-4-[(4-methoxyphenyl)methyl]-2H,3H,4H-pyrido[3,2-b][1,4]oxazin-3-one 1c (94 mg, 0.23 mmol, 1 eq) in DCE (15 mL) was stirred at room temperature for 30 min. NaBH(OAc)₃ (150 mg, 0.71 mmol, 3 eq) was then added and stirring was continued at room temperature for 17 h. The mixture was diluted with DCM (30 mL) and washed with water, brine, dried over Na₂SO₄, filtered and concentrated under reduced pressure to give rel-1-{2-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]ethyl}-7-(pyridin-3-yloxy)-1,2-dihydro-1,8-naphthyridin-2-one 7c (200 mg, 129%), which was used directly in the next step without purification. TLC: R_{f}=0.45 (silica gel, DCM/MeOH=20:1, v/v). LC-MS (Method C) 676.3 [M+H]⁺; RT 3.12 min.

### d) rel-1-{2-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]ethyl}-7-(pyridin-3-yloxy)-1,2-dihydro-1,8-naphthyridin-2-one

To a solution of rel-1-{2-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]ethyl}-7-(pyridin-3-yloxy)-1,2-dihydro-1,8-naphthyridin-2-one 7c (200 mg crude, assumed 0.23 mmol, 1 eq) in DCM (10 mL) was added TFA (2.0 mL) followed by CF₃SO₃H (1.0 mL) and the mixture was stirred at room temperature for 1 h. Water (40 mL) was added and the mixture adjusted to pH 8-9 with a saturated aqueous Na₂CO₃ solution, then extracted with DCM (20 mL×3). The combined organic extracts were washed with brine (20 mL), dried over Na₂SO₄, filtered and concentrated under reduced pressure. The residue was purified by preparative TLC (DCM/MeOH, 10:1, v/v) to give rel-1-{2-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]ethyl}-7-(pyridin-3-yloxy)-1,2-dihydro-1,8-naphthyridin-2-one 7 (30 mg, 25%) as a white solid. TLC: R_{f}=0.25 (silica gel, DCM/MeOH=10:1, v/v).). LC-MS (Method D) 556.2 [M+H]⁺; RT 2.40 min. 1H NMR (Method E) (DMSO-d6): δ ppm 11.3 (s, 1H), 8.59 (s, 1H), 8.51 (s, 1H), 8.26 (d, J=6.8 Hz, 1H), 7.93 (d, J=7.6 Hz, 1H), 7.79 (d, J=6.0 Hz, 1H), 7.56 (m, 1H), 7.41 (d, J=6.0 Hz, 1H), 7.17 (d, J=7.6 Hz, 1H), 7.08 (d, J=6.0 Hz, 1H), 6.56 (d, J=8.4 Hz, 1H), 4.66 (s, 2H), 4.38 (m, 1H), 4.04 (m, 3H), 3.49 (m, 1H), 2.76 (m, 1H), 2.72-2.52 (m, 4H), 2.30 (m, 2H).

### Example 8:—rel-1-{2-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]ethyl}-7-(3-aminopropoxy)-1,2-dihydro-1,8-naphthyridin-2-one

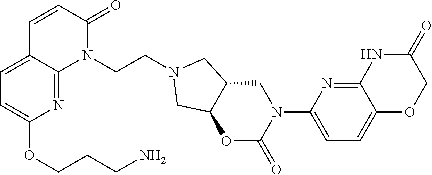 [SMILES: NCCCOc1ccc2ccc(=O)n(CCN3C[C@@H]4CN(c5ccc6c(n5)NC(=O)CO6)C(=O)O[C@H]4C3)c2n1]

### a) tert-butyl N-(3-{[8-(2,2-diethoxyethyl)-7-oxo-7,8-dihydro-1,8-naphthyridin-2-yl]oxy}propyl)carbamate 8a

To a solution of 7-chloro-1-(2,2-diethoxyethyl)-1,2-dihydro-1,8-naphthyridin-2-one 5a (595 mg, 2.0 mmol) and tert-butyl N-(3-hydroxypropyl)carbamate (1.05 g, 6.0 mmol) in DMF (15 mL) was added a solution of t-BuOK (784 mg, 7.0 mmol) in dry THF (25 mL) and the mixture was stirred at room temperature overnight. The mixture was diluted with H₂O (100 mL), extracted with EtOAc (100 mL×3) and the combined organic extracts were washed with H₂O (100 mL×2), brine (100 mL) and concentrated under reduced pressure. The residue was purified by chromatography (petroleum ether/EtOAc, 3:1, v/v) to give a colourless oil of tert-butyl N-(3-{[8-(2,2-diethoxyethyl)-7-oxo-7,8-dihydro-1,8-naphthyridin-2-yl]oxy}propyl)carbamate 8a (540 mg). TLC: R_{f}=0.30 (silica gel, petroleum ether/EtOAc, 2:1, v/v)¹H NMR (Method E) (DMSO-d6): δ ppm 8.04 (d, J=8.4 Hz, 1H), 7.86 (d, J=9.4 Hz, 1H), 6.89 (m, 1H), 6.71 (d, J=8.4 Hz, 1H), 6.50 (d, J=9.4 Hz, 1H), 5.03 (t, J=5.7 Hz, 1H), 4.48 (d, J=5.7 Hz, 2H), 4.38 (m, 2H), 3.65 (m, 2H), 3.37 (m, 2H), 3.09 (m, 2H), 1.90 (m, 2H), 1.36 (s, 9H), 0.98 (t, J=7.0 Hz, 6H).

### b) tert-butyl N-(3-{[7-oxo-8-(2-oxoethyl)-7,8-dihydro-1,8-naphthyridin-2-yl]oxy}propyl)carbamate 8b

To a solution of tert-butyl N-(3-{[8-(2,2-diethoxyethyl)-7-oxo-7,8-dihydro-1,8-naphthyridin-2-yl]oxy}propyl)carbamate 8a (540 mg, 1.24 mmol) in THF (10 mL) was added 1 M HCl (6.0 mL, 6.0 mmol) and the mixture was stirred at room temperature for 20 h. The mixture was adjusted to pH 8-9 with a saturated aqueous NaHCO₃ solution and extracted with EtOAc (20 mL×3). The combined organic extracts were washed with H₂O (50 mL), brine (50 mL), dried over Na₂SO₄, filtered and concentrated under reduced pressure. The residue was purified by chromatography (petroleum ether/EtOAc, 5:1 to 1:1, v/v) to give a white solid of tert-butyl N-(3-{[7-oxo-8-(2-oxoethyl)-7,8-dihydro-1,8-naphthyridin-2-yl]oxy}propyl)carbamate 8b (250 mg, 56%). TLC: R_{f}=0.14 (silica gel, petroleum ether/EtOAc=2:1, v/v). LC-MS (Method C) 362.3 [M+H]⁺, RT 3.38 min. ¹H-NMR (Method E) (CDCl₃) δ ppm: 9.65 (s, 1H), 7.77 (d, J=8.4 Hz, 1H), 7.65 (d, J=9.4 Hz, 1H), 6.63 (d, J=9.6 Hz, 1H), 6.62 (d, J=8.4 Hz, 1H), 5.20 (s, 2H), 4.32 (t, J=6.2 Hz, 2H), 3.29 (m, 2H), 1.95 (m, 2H), 1.43 (s, 9H).

### c) rel-tert-butyl N-{3-[(8-{2-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]ethyl}-7-oxo-7,8-dihydro-1,8-naphthyridin-2-yl)oxy]propyl}carbamate 8c

A mixture of tert-butyl N-(3-{[7-oxo-8-(2-oxoethyl)-7,8-dihydro-1,8-naphthyridin-2-yl]oxy}propyl)carbamate 8b (66 mg, 0.18 mmol, 1 eq) and rel-6-[(4aS*,7aR*)-2-oxo-octahydropyrrolo[3,4-e][1,3]oxazin-3-yl]-4-[(4-methoxyphenyl)methyl]-2H,3H,4H-pyrido[3,2-b][1,4]oxazin-3-one 1c (75 mg, 0.18 mmol, 1 eq) in DCE (20 mL) was stirred at room temperature for 30 min. NaBH(OAc)₃ (191 mg, 0.90 mmol, 5 eq) was then added and stirring was continued at room temperature for 17 h. The mixture was diluted with a saturated aqueous NaHCO₃ solution and extracted with EtOAc (20 mL×3). The combined organic extracts were washed with water, brine, dried over Na₂SO₄, filtered and concentrated under reduced pressure to give rel-tert-butyl N-{3-[(8-{2-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]ethyl}-7-oxo-7,8-dihydro-1,8-naphthyridin-2-yl)oxy]propyl}carbamate 8c (160 mg), which was used in the next step without further purification. TLC: R_{f}=0.30 (silica gel, DCM/MeOH=20:1, v/v). LC-MS (Method C) 756.3 [M+H]⁺, RT 3.70 min.

### d) rel-1-{2-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]ethyl}-7-(3-aminopropoxy)-1,2-dihydro-1,8-naphthyridin-2-one 8

To a solution of rel-tert-butyl N-{3-[(8-{2-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]ethyl}-7-oxo-7,8-dihydro-1,8-naphthyridin-2-yl)oxy]propyl}carbamate 8c (136 mg, 0.18 mmol, 1 eq) in DCM (10 mL) was added TFA (2.0 mL, 26.1 mmol) and the mixture was stirred at room temperature for 4 h. CF₃SO₃H (1.0 mL, 11.3 mmol) was then added and stirring was continued at room temperature for a further 1 h. The mixture was adjusted to pH 8-9 with a saturated aqueous K₂CO₃ solution (40 mL) and then extracted with DCM/IPA (3:1, 20 mL x 3). The combined organic extracts were washed with water, brine, dried over Na₂SO₄, filtered and concentrated under reduced pressure. The residue was purified by preparative TLC (DCM/MeOH/NH₄OH, 100:10:2, v/v) to give a white solid (15 mg, 20%) which was dissolved in DMSO (5 mL) and loaded onto a Isolute SPE SCX-2 cartridge (1 g, prewashed with MeOH). The cartridge was washed with DMSO, acetone, H₂O and MeOH followed by NH₃ in MeOH (3M). The ammoniacal fraction was concentrated under reduced pressure and dried in vacuo at 50° C. to give rel-1-{2-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]ethyl}-7-(3-aminopropoxy)-1,2-dihydro-1,8-naphthyridin-2-one 8 (6 mg, 6.2%). LC-MS (Method C) 536.3 [M+H]⁺, RT 0.90 min. ¹H NMR (Method E) (DMSO-d6): δ ppm 8.06 (d, J=8.3 Hz, 1H), 7.87 (d, J=9.4 Hz, 1H), 7.41 (d, J=8.5 Hz, 1H), 7.17 (d, J=8.4 Hz, 1H), 6.73 (d, J=8.4 Hz, 1H), 6.51 (d, J=9.4 Hz, 1H), 4.66 (s, 2H), 4.55 (m, 1H), 4.51-4.38 (m, 4H), 4.04 (dd, J=10.7, 5.4 Hz, 1H), 3.56 (t, J=11.4 Hz, 1H), 3.10-3.25 (m, 3H), 3.01-2.93 (m, 1H), 2.93-2.79 (m, 2H), 2.78-2.59 (m, 3H), 1.87 (m, 2H).

### Example 9:—rel-(4aS*,7aR*-6-(2-{7-methoxy-4-oxo-4H,5H-[1,2]oxazolo[3,4-c]quinolin-5-yl}ethyl)-3-{3-oxo-2H,3H,4H-pyrido[3,2-b][1,4]oxazin-6-yl}-octahydropyrrolo[3,4-e][1,3]oxazin-2-one

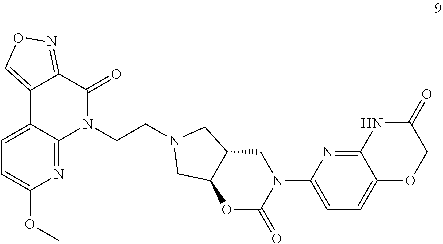 [SMILES: COc1ccc2c3conc3c(=O)n(CCN3C[C@@H]4CN(c5ccc6c(n5)NC(=O)CO6)C(=O)O[C@H]4C3)c2n1]

### a) 5-(2-hydroxyethyl)-7-methoxy-4H,5H-[1,2]oxazolo[3,4-c]quinolin-4-one 9a

A mixture of 7-methoxy-4H,5H-[1,2]oxazolo[3,4-c]quinolin-4-one (WO 2016024096 A1) (1.1 g, 5.1 mmol, 1 eq), 1,2-dibromoethane (5.2 g, 27.7 mmol, 5.4 eq) and Cs₂CO₃ (8.5 g, 26.1 mmol, 5.1 eq) in NMP (80 mL) was heated at 45° C. for 4 h. The mixture was then poured into water and extracted with EtOAc. The organic layer was washed with brine, dried over Na₂SO₄, filtered and concentrated under reduced pressure. The residue was purified by silica gel chromatography (petroleum ether/EtOAc, 3:1 to 1:1, v/v) to give 5-(2-hydroxyethyl)-7-methoxy-4H,5H-[1,2]oxazolo[3,4-c]quinolin-4-one 9a (700 mg, 54%) as a grey solid. TLC: R_{f}=0.27 (silica gel, EtOAc/pet. ether=1:1, v/v). LC-MS (Method C) 261.1 [M+H]⁺; RT 1.72 min. ¹H NMR (Method E) (DMSO-d6): δ ppm 9.92 (s, 1H), 7.94 (d, J=8.4 Hz, 1H), 7.16 (d, J=2.0 Hz, 1H), 6.95 (dd, J=8.4, 2.0 Hz, 1H), 4.96 (t, J=6.0 Hz, 1H), 4.33 (t, J=6.4 Hz, 2H), 3.87 (s, 3H), 3.71 (dt, J=6.0, 6.4 Hz, 2H). ¹³C NMR (Method E) (DMSO-d6): δ ppm 160.6, 156.4, 154.9, 150.9, 138.8, 127.4, 117.3, 109.9, 106.5, 102.9, 58.3, 56.0, 44.7.

### b) 2-{7-methoxy-4-oxo-4H,5H-[1,2]oxazolo[3,4-c]quinolin-5-yl}acetaldehyde 9b

A mixture of 5-(2-hydroxyethyl)-7-methoxy-4H,5H-[1,2]oxazolo[3,4-c]quinolin-4-one 9a (150 mg, 0.38 mmol, 1 eq) and PCC (800 mg, 3.7 mmol, 9.7 eq) in DCM (20 mL) was stirred at room temperature for 6 h. The mixture was filtered through silica gel and the filtrate was concentrated under reduced pressure to afford 2-{7-methoxy-4-oxo-4H,5H-[1,2]oxazolo[3,4-c]quinolin-5-yl}acetaldehyde 9b (80 mg) as a grey solid, which was used directly in the next step without further purification. TLC: R_{f}=0.5 (silica gel, EA:PE=1:1, v/v). LC-MS (Method C) 259.0 [M+H]⁺; RT 3.16 min.

### c) rel-(4aS*,7aR*)-6-(2-{7-methoxy-4-oxo-4H,5H-[1,2]oxazolo[3,4-c]quinolin-5-yl}ethyl)-3-{4-[(4-methoxyphenyl)methyl]-3-oxo-2H,3H,4H-pyrido[3,2-b][1,4]oxazin-6-yl}-octahydropyrrolo[3,4-e][1,3]oxazin-2-one 9c

To a suspension of 2-{7-methoxy-4-oxo-4H,5H-[1,2]oxazolo[3,4-c]quinolin-5-yl}acetaldehyde 9b (55 mg, 0.21 mmol, 1 eq) in DCM (20 mL) at ambient temperature was added rel-6-[(4aS*,7aR*)-2-oxo-octahydropyrrolo[3,4-e][1,3]oxazin-3-yl]-4-[(4-methoxyphenyl)methyl]-2H,3H,4H-pyrido[3,2-b][1,4]oxazin-3-one 1c (85 mg, 0.21 mmol, 1 eq) followed by NaBH(OAc)₃ (250 mg, 1.18 mmol, 5.5 eq) and the resulting mixture was stirred for 18 h. The mixture was then adjusted to pH 8-9 with a saturated aqueous Na₂CO₃ solution and extracted with DCM. The organic layer was washed with brine, dried over Na₂SO₄, filtered and concentrated under reduced pressure. The residue was purified by silica gel chromatography (DCM/MeOH, 30:1 to 15:1, v/v) to give rel-(4aS*,7aR*)-6-(2-{7-methoxy-4-oxo-4H,5H-[1,2]oxazolo[3,4-c]quinolin-5-yl}ethyl)-3-{4-[(4-methoxyphenyl)methyl]-3-oxo-2H,3H,4H-pyrido[3,2-b][1,4]oxazin-6-yl}-octahydropyrrolo[3,4-e][1,3]oxazin-2-one 9c (65 mg, 47%) as a white solid. TLC: R_{f}=0.31 (silica gel, DCM/MeOH=15:1, v/v). LC-MS (Method D) 653.2 [M+H]⁺; RT 3.61 min.

### d) rel-(4aS*,7aR*)-6-(2-{7-methoxy-4-oxo-4H,5H-[1,2]oxazolo[3,4-c]quinolin-5-yl}ethyl)-3-{3-oxo-2H,3H,4H-pyrido[3,2-b][1,4]oxazin-6-yl}-octahydropyrrolo[3,4-e][1,3]oxazin-2-one 9

To a solution of rel-(4aS*,7aR*)-6-(2-{7-methoxy-4-oxo-4H,5H-[1,2]oxazolo[3,4-c]quinolin-5-yl}ethyl)-3-{4-[(4-methoxyphenyl)methyl]-3-oxo-2H,3H,4H-pyrido[3,2-b][1,4]oxazin-6-yl}-octahydropyrrolo[3,4-e][1,3]oxazin-2-one 9c (60 mg, 0.09 mmol, 1 eq) in DCM (10 mL) was added TFA (1.5 mL, 19.5 mmol) followed by CF₃SO₃H (0.4 mL, 4.5 mmol) and the resulting mixture was stirred at room temperature for 1 h. The reaction was quenched with MeOH (1 mL) and the mixture was adjusted to pH 10 with a saturated aqueous Na₂CO₃ solution and extracted with DCM (100 mL). The organic layer was washed with brine, dried over Na₂SO₄, filtered and concentrated under reduced pressure. The residue was purified by silica gel chromatography (DCM/MeOH, 10:1, v/v) to afford rel-(4aS*,7aR*)-6-(2-{7-methoxy-4-oxo-4H,5H-[1,2]oxazolo[3,4-c]quinolin-5-yl}ethyl)-3-{3-oxo-2H,3H,4H-pyrido[3,2-b][1,4]oxazin-6-yl}-octahydropyrrolo[3,4-e][1,3]oxazin-2-one 9 (20 mg, 41%) as a pale yellow solid. TLC: R_{f}=0.39 (silica gel, DCM/MeOH=10:1, v/v). LC-MS (Method D) 533.2 [M+H]⁺; RT 2.72 min. ¹H NMR (Method E) (DMSO-d6): δ ppm 11.2 (s, 1H), 9.95 (s, 1H), 7.99 (d, J=8.4 Hz, 1H), 7.40 (d, J=8.4 Hz, 1H), 7.16 (d, J=8.4 Hz, 1H), 7.03-6.99 (m, 2H), 4.65 (s, 2H), 4.55 (m, 1H), 4.39 (m, 2H), 4.03 (m, 1H), 3.90 (s, 3H), 3.55 (m, 1H), 3.18 (m, 2H), 2.99-2.89 (m, 3H), 2.69 (m, 1H), 2.45 (m, 1H).

### Example 10:—rel-1-{2-[(4aS*,7aR*-2-oxo-3-{3-oxo-2H,3H,4H-pyrido[3,2-b][1,4]oxazin-6-yl}-octahydropyrrolo[3,4-e][1,3]oxazin-6-yl]ethyl}-2-oxo-1,2-dihydroquinoline-7-carbonitrile

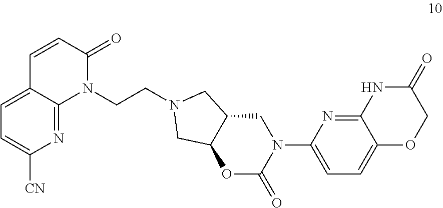 [SMILES: N#Cc1ccc2ccc(=O)n(CCN3C[C@@H]4CN(c5ccc6c(n5)NC(=O)CO6)C(=O)O[C@H]4C3)c2n1]

### a) rel-1-{2-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]ethyl}-2-oxo-1,2-dihydroquinoline-7-carbonitrile 10a

To a solution of 2-oxo-1-(2-oxoethyl)-1,2-dihydroquinoline-7-carbonitrile (56 mg, 0.29 mmol) (prepared as described in J. Med. Chem (2014) 57 (11), 4889-4905) and molecular sieves (4 Å) in DCM (5 mL) was added rel-6-[(4aS*,7aR*)-2-oxo-octahydropyrrolo[3,4-e][1,3]oxazin-3-yl]-4-[(4-methoxyphenyl)methyl]-2H,3H,4H-pyrido[3,2-b][1,4]oxazin-3-one 1c (110 mg, 0.29 mmol). After 2 h, NaBH(OAc)₃ (170 mg, 0.80 mmol) was added and the mixture left to stir at room temperature for a further 2 h. The reaction was diluted with saturated aqueous NaHCO₃ and extracted with DCM (20 mL), dried over MgSO₄, filtered and concentrated under reduced pressure to give a brown solid of rel-1-{2-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]ethyl}-2-oxo-1,2-dihydroquinoline-7-carbonitrile 10a (60 mg, 36%) LC-MS (Method A) 607.2 [M+H]⁺, RT 2.79 min.

### b) rel-1-{2-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]ethyl}-2-oxo-1,2-dihydroquinoline-7 carbonitrile 10

To a solution of rel-1-{2-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]ethyl}-2-oxo-1,2-dihydroquinoline-7-carbonitrile 10a (60 mg, 0.10 mmol) in DCM (5 mL) was added TFA (0.4 mL, 5.2 mmol) and CF₃SO₃H (0.2 mL, 2.2 mmol). After 1 h the reaction was quenched with MeOH (5 mL). Saturated aqueous NaHCO₃ (50 mL) was added, adjusting the pH to 8 and the mixture was extracted with DCM (50 mL×2). The combined organic extracts were washed with water (50 ml), dried over Na₂SO₄, filtered and concentrated under reduced pressure then purified by silica gel chromatography (0-10% MeOH in EtOAc) to give rel-1-{2-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]ethyl}-2-oxo-1,2-dihydroquinoline-7 carbonitrile 10 (12 mg, 25%) as a white solid. LC-MS (Method B) 487.1 [M+H]⁺; RT 5.17 min. ¹H NMR (Method C) (DMSO-d6): δ ppm 10.80 (s, 1H), 8.06 (s, 1H), 7.99 (d, J=9.6 Hz, 1H), 7.91 (d, J=8.4 Hz, 1H), 7.65-7.60 (m, 1H), 7.37 (d, J=8.4 Hz, 1H), 7.21 (d, J=8.4 Hz, 1H), 6.79 (d, J=9.6 Hz, 1H), 4.64 (s, 2H), 4.63-4.56 (m, 1H), 4.54-4.46 (m, 2H), 4.10 (dd, J=11.4, 5.6 Hz, 1H), 3.62 (t, J=11.4 Hz, 1H), 3.24-3.17 (m, 2H), 3.24 (m, 1H), 2.61-2.57 (m, 1H), 2.37 (m, 1H), 1.31-1.27 (m, 2H).

### Example 11:—rel-(4aS*,7aR*)-6-(3-{2-methoxy-7-oxo-7H,8H-pyrido[2,3-d]pyrimidin-8-yl}propyl)-3-{3-oxo-2H,3H,4H-pyrido[3,2-b][1,4]oxazin-6-yl}-octahydropyrrolo[3,4-e][1,3]oxazin-2-one

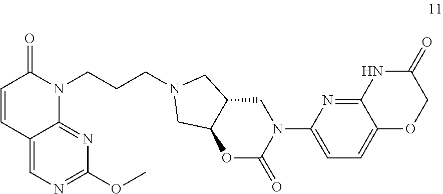 [SMILES: COc1ncc2ccc(=O)n(CCCN3C[C@@H]4CN(c5ccc6c(n5)NC(=O)CO6)C(=O)O[C@H]4C3)c2n1]

### a) 5-bromo-N-(3,3-diethoxypropyl)-2-(methylsulfanyl)pyrimidin-4-amine 11a

5-bromo-4-chloro-2-(methylsulfanyl)pyrimidine (9.2 g, 38.4 mmol), K₂CO₃ (6.9 g, 49.9 mmol) and 3,3-diethoxypropan-1-amine (5.65 g, 38.4 mmol) were added to DMF (80 mL). The reaction was stirred for 3 h then quenched with H₂O (100 mL), extracted with diethyl ether (2×75 mL) and dried over MgSO₄. After filtration the filtrate was concentrated under reduced pressure to afford 5-bromo-N-(3,3-diethoxypropyl)-2-(methylsulfanyl)pyrimidin-4-amine 11a (13.2 g, 98%) as a yellow gum. LC-MS (Method A) 351.9 [M+H]⁺; RT 3.32 min.

### b) Butyl (2E)-3-{4-[(3,3-diethoxypropyl)amino]-2-(methylsulfanyl)pyrimidin-5-yl}prop-2-enoate 11b

5-bromo-N-(3,3-diethoxypropyl)-2-(methylsulfanyl)pyrimidin-4-amine 11a (13.8 g, 39.4 mmol), triethylamine (20 mL, 143 mmol), butyl acrylate (22.59 mL, 157.59 mmol) and tetrakis(triphenylphosphine)palladium(0) (4.9 g, 4.29 mmol) were combined and stirred at 100° C. for 18 h. Butyl acrylate (5 mL, 34.88 mmol) and tetrakis(triphenylphosphine)palladium(0) (500 mg, 0.44 mmol) were added and the reaction stirred at reflux for a further 18 h. After cooling, the reaction was evaporated to dryness under reduced pressure and H₂O (150 mL) added. The mixture was extracted with Et₂O (3×150 mL), the organic extracts combined, washed with brine (100 mL) and dried over MgSO₄. The solvent was evaporated and purified by silica gel chromatography using 0-50% EtOAc in heptane to afford butyl (2E)-3-{4-[(3,3-diethoxypropyl)amino]-2-(methylsulfanyl)pyrimidin-5-yl}prop-2-enoate 11b (5.7 g, 36% yield) as a very pale yellow oil which very slowly solidified. ¹H NMR (Method C) (CDCl₃): δ ppm 8.10 (s, 1H), 7.40 (d, J=15.9 Hz, 1H), 6.27 (d, J=15.9 Hz, 1H), 6.12-6.00 (m, 1H), 4.66 (t, J=5.2 Hz, 1H), 4.20 (t, J=6.7 Hz, 2H), 3.75-3.65 (m, 4H), 3.57-3.50 (m, 2H), 2.52 (s, 3H), 2.00-1.95 (m, 2H), 1.71-1.65 (m, 2H), 1.46-1.39 (m, 2H), 1.27-1.22 (t, J=7.4 Hz, 6H), 0.90 (t, J=7.4 Hz, 3H).

### c) 8-(3,3-diethoxypropyl)-2-(methylsulfanyl)-7H,8H-pyrido[2,3-d]pyrimidin-7-one 11c NaOMe (0.8 g, 14.8 mmol) was Added to a Solution of Butyl

(2E)-3-{4-[(3,3-diethoxypropyl)amino]-2-(methylsulfanyl)pyrimidin-5-yl}prop-2-enoate 11b (5.7 g, 14.34 mmol) in MeOH (20 mL) and heated at reflux for 48 h. The mixture was evaporated to half its volume, quenched with saturated aqueous NH₄Cl (100 mL) and extracted with Et₂O (2×75 mL). The combined organic extracts were dried over MgSO₄, filtered and concentrated under reduced pressure. The solid was purified by silica gel chromatography using 0-50% ethyl acetate in Et₂O to afford 8-(3,3-diethoxypropyl)-2-(methylsulfanyl)-7H,8H-pyrido[2,3-d]pyrimidin-7-one 11c (3.1 g, 66%) as a white solid. ¹H NMR (Method C) (CDCl₃): δ ppm 8.59 (s, 1H), 7.58 (d, J=9.5 Hz, 1H), 6.62 (d, J=9.4 Hz, 1H), 4.79 (t, J=5.6 Hz, 1H), 4.54-4.49 (m, 2H), 3.71-3.64 (m, 2H), 3.52-3.46 (m 2H), 2.65 (s. 3H), 2.11-2.05 (m 2H), 1.20-1.15 (t, J=7.4 Hz, 6H).

### d) 8-(3,3-diethoxypropyl)-2-methoxy-7H,8H-pyrido[2,3-d]pyrimidin-7-one 11d

NaOMe (0.84 mL, 15.46 mmol) was added to a solution of 8-(3,3-diethoxypropyl)-2-(methylsulfanyl)-7H,8H-pyrido[2,3-d]pyrimidin-7-one 11c (1.0 g, 3.09 mmol) in MeOH (20 mL) and the mixture heated at 140° C. for 17 h. The reaction was cooled, quenched with H₂O (75 mL) and extracted with Et₂O (2×75 mL). The combined organic extracts were dried over MgSO₄ and concentrated under reduced pressure to afford 8-(3,3-diethoxypropyl)-2-methoxy-7H,8H-pyrido[2,3-d]pyrimidin-7-one 11d (0.86 g, 90%) as a yellow gum which was used without further purification. LC-MS (Method A) 308.1 [M+H]⁺, RT 2.29 min.

### e) 3-{2-methoxy-7-oxo-7H,8H-pyrido[2,3-d]pyrimidin-8-yl}propanal 11e

Aqueous HCl (3.0 M, 20 mL) was added to a solution of 8-(3,3-diethoxypropyl)-2-methoxy-7H,8H-pyrido[2,3-d]pyrimidin-7-one 11d (860 mg, 2.8 mmol) in THF (20 mL) and heated at reflux for 3 h. The mixture was quenched with H₂O (30 mL), basified with solid K₂CO₃ and extracted with Et₂O/EtOAc (1:1 v/v, 2×75 ml). The combined organic extracts were dried over MgSO₄ and concentrated under reduced pressure to afford an off white solid. This solid was triturated with Et₂O, filtered and dried to give 3-{2-methoxy-7-oxo-7H,8H-pyrido[2,3-d]pyrimidin-8-yl}propanal 11e (423 mg, 65%) as a white solid. LC-MS (Method A) 234.1 [M+H]⁺, RT 1.48 min.

### f) rel-(4aS*,7aR*)-6-(3-{2-methoxy-7-oxo-7H,8H-pyrido[2,3-d]pyrimidin-8-yl}propyl)-3-{4-[(4-methoxyphenyl)methyl]-3-oxo-2H,3H,4H-pyrido[3,2-b][1,4]oxazin-6-yl}-octahydropyrrolo[3,4-e][1,3]oxazin-2-one 11f

3-(2-methoxy-7-oxo-pyrido[2,3-d]pyrimidin-8-yl)propanal 11e (62 mg, 0.27 mmol), rel-6-[(4aS*,7aR*)-2-oxo-octahydropyrrolo[3,4-e][1,3]oxazin-3-yl]-4-[(4-methoxyphenyl)methyl]-2H,3H,4H-pyrido[3,2-b][1,4]oxazin-3-one 1c (100 mg, 0.24 mmol) and molecular sieves (3 Å) were added to DCM (20 mL) and stirred for 4 h. To this was added NaBH(OAc)₃ (77 mg, 0.37 mmol) and the mixture stirred for 2 h. The reaction was quenched with saturated aqueous K₂CO₃ (40 mL) and extracted with DCM (2×40 mL). The organic extracts were combined, dried over MgSO₄ and concentrated under reduced pressure to yield rel-(4aS*,7aR*)-6-(3-{2-methoxy-7-oxo-7H,8H-pyrido[2,3-d]pyrimidin-8-yl}propyl)-3-{4-[(4-methoxyphenyl)methyl]-3-oxo-2H,3H,4H-pyrido[3,2-b][1,4]oxazin-6-yl}-octahydropyrrolo[3,4-e][1,3]oxazin-2-one 11f (150 mg, 88%) as a yellow foam. LC-MS (Method A) 628.2 [M+H]⁺, RT 2.54 min.

### g) rel-(4aS*,7aR*)-6-(3-{2-methoxy-7-oxo-7H,8H-pyrido[2,3-d]pyrimidin-8-yl}propyl)-3-{3-oxo-2H,3H,4H-pyrido[3,2-b][1,4]oxazin-6-yl}-octahydropyrrolo[3,4-e][1,3]oxazin-2-one 11

To a stirred solution of rel-(4aS*,7aR*)-6-(3-{2-methoxy-7-oxo-7H,8H-pyrido[2,3-d]pyrimidin-8-yl}propyl)-3-{4-[(4-methoxyphenyl)methyl]-3-oxo-2H,3H,4H-pyrido[3,2-b][1,4]oxazin-6-yl}-octahydropyrrolo[3,4-e][1,3]oxazin-2-one 11f (150 mg, 0.24 mmol) in DCM (5 mL) was added TFA (0.92 mL, 11.95 mmol) followed by CF₃SO₃H (0.2 mL, 2.39 mmol). The reaction was stirred for 3 h then quenched with MeOH (10 mL). K₂CO₃ was added adjusting the pH to 8, then the mixture diluted with H₂O (25 mL) and stirred for 30 min. The mixture was extracted with DCM (2×25 mL) and the combined organic extracts dried over MgSO₄. The filtrate was concentrated under reduced pressure to give a yellow residue. MeOH and DCM were added to dissolve the residue and the solution was passed through an Isolute SCX-2 cartridge. The cartridge was washed with MeOH and the product released using NH₃ in MeOH (3.5 M). The isolated foam was purified by silica gel chromatography using 0-20% MeOH in EtOAc. The residue was then dissolved in DCM (2 mL) and treated with Et₂O (20 mL) to afford a precipitate which was filtered and dried to give rel-(4aS*,7aR*)-6-(3-{2-methoxy-7-oxo-7H,8H-pyrido[2,3-d]pyrimidin-8-yl}propyl)-3-{3-oxo-2H,3H,4H-pyrido[3,2-b][1,4]oxazin-6-yl}-octahydropyrrolo[3,4-e][1,3]oxazin-2-one 11 (28 mg, 22%) as a white solid. LC-MS (Method A) 508.2 [M+H]⁺, RT 1.69 min. ¹H NMR (Method C) (CDCl₃): δ ppm 8.66 (s, 1H), 8.12 (s, 1H), 7.62 (d, J=9.5 Hz, 1H), 7.38 (d, J=8.6 Hz, 1H), 7.29 (d, J=8.5 Hz, 1H), 6.59 (d, J=9.4 Hz, 1H), 4.65 (s, 2H), 4.52-4.37 (m, 3H), 4.17 (dd, J=11.1, 5.5 Hz, 1H), 4.10 (s, 3H), 3.58 (t, J=11.6 Hz, 1H), 3.13 (ddd, J=8.9, 7.4, 1.7 Hz, 2H), 2.93 (t, J=9.0 Hz, 1H), 2.79 (m, 2H), 2.71-2.63 (m, 1H), 2.53-2.45 (m, 1H), 1.98-1.88 (m, 2H).

### Example 12:—1-{2-[(4aR,7aS)-2-oxo-3-{3-oxo-2H,3H,4H-pyrido[3,2-b][1,4]oxazin-6-yl}-octahydropyrrolo[3,4-e][1,3]oxazin-6-yl]ethyl}-2-oxo-1,2-dihydroquinoline-7-carbonitrile

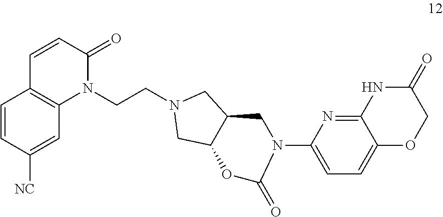 [SMILES: N#Cc1ccc2ccc(=O)n(CCN3C[C@H]4CN(c5ccc6c(n5)NC(=O)CO6)C(=O)O[C@@H]4C3)c2c1]

### a) tert-butyl(4aR,7aS)-2-oxo-octahydropyrrolo[3,4-e][1,3]oxazine-6-carboxylate 12a

A solution of tert-butyl (3R,4S)-3-(aminomethyl)-4-hydroxypyrrolidine-1-carboxylate (2.1 g, 9.71 mmol) (as prepared in WO200809700 A1) and CDI (1.89 g, 11.65 mmol) in 1,4-dioxane (40 mL) was heated to 100° C. for 24 h. The reaction was cooled and concentrated under reduced pressure. Aqueous HCl (2 M, 50 mL) was added then the mixture extracted with EtOAc (3×30 mL), dried (MgSO₄) and concentrated under reduced pressure to give tert-butyl (4aR,7aS)-2-oxo-octahydropyrrolo[3,4-e][1,3]oxazine-6-carboxylate 12a (510 mg, 23%) as a white solid.

### b) tert-butyl (4aS,7aS)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazine-6-carboxylate 12b

A solution of tert-butyl (4aR,7aS)-2-oxo-octahydropyrrolo[3,4-e][1,3]oxazine-6-carboxylate 12a (510 mg, 2.10 mmol), 6-bromo-4-[(4-methoxyphenyl)methyl]-2H,3H,4H-pyrido[3,2-b][1,4]oxazin-3-one (prepared as described in WO2014108832) (735 mg, 2.10 mmol), Brett Phos G3 pre catalyst (190 mg, 0.21 mmol), K₃PO₄ (1.34 g, 6.31 mmol) in 1,4-dioxane (50 mL) was heated to 100° C. for 5 h. The reaction was cooled and H₂O (50 mL) added then extracted with EtOAc (3×30 mL), dried (MgSO₄), filtered and concentrated under reduced pressure. The residue was purified by silica gel chromatography 0-100% EtOAc in pet. ether to give tert-butyl (4aS,7aS)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazine-6-carboxylate 12b (510 mg, 47%) as a yellow oil. LC-MS (Method A) 511.1 [M+H]⁺, RT 3.14 min.

### c) (4aS,7aS)-2-oxo-3-{3-oxo-2H,3H,4H-pyrido[3,2-b][1,4]oxazin-6-yl}-octahydropyrrolo[3,4-e][1,3]oxazin-6-ium trifluoroacetate, 12c

A solution of (4aS,7aS)-2-oxo-3-{3-oxo-2H,3H,4H-pyrido[3,2-b][1,4]oxazin-6-yl}-octahydropyrrolo[3,4-e][1,3]oxazine-6-carboxylate 12b (599 mg, 1.17 mmol), TFA (2 mL) and CF₃SO₃H (0.2 mL) in DCM (10 mL) was stirred at room temperature for 30 min. The reaction was quenched with MeOH (5 mL) and then concentrated under reduced pressure. The residue was triturated with DCM and diethyl ether then filtered to give (4aR,7aS)-2-oxo-3-{3-oxo-2H,3H,4H-pyrido[3,2-b][1,4]oxazin-6-yl}-octahydropyrrolo[3,4-e][1,3]oxazin-6-ium trifluoroacetate 12c (210 mg, 52%) as a white solid. LC-MS (Method A) 291.2 [M+H]⁺, RT 1.17 min.

### d) 1-{2-[(4aR,7aS)-2-oxo-3-{3-oxo-2H,3H,4H-pyrido[3,2-b][1,4]oxazin-6-yl}-octahydropyrrolo[3,4-e][1,3]oxazin-6-yl]ethyl}-2-oxo-1,2-dihydroquinoline-7-carbonitrile, 12

A solution of (4aR,7aS)-2-oxo-3-{3-oxo-2H,3H,4H-pyrido[3,2-b][1,4]oxazin-6-yl}-octahydropyrrolo[3,4-e][1,3]oxazin-6-ium trifluoroacetate 12c (210 mg, 0.52 mmol), 2-oxo-1-(2-oxoethyl)-1,2-dihydroquinoline-7-carbonitrile (93 mg, 0.47 mmol) (prepared as described in J. Med. Chem (2014) 57 (11), 4889-4905) and triethylamine (0.35 mL 1.51 mmol) were stirred with molecular sieves (4 Å) in DCM (5 mL) for 2 h. NaBH(OAc)₃ (330 mg, 1.51 mmol) was added and the mixture left to stir at room temperature for a further 2 h. Saturated aqueous NaHCO₃ (20 mL) was added and the mixture extracted with DCM (20 mL), dried (MgSO₄), filtered and concentrated under reduced pressure. The residue was purified via silica gel chromatography 0-20% MeOH in EtOAc and then dissolved in a DCM (1 mL) and triturated with diethyl ether to give 1-{2-[(4aR,7aS)-2-oxo-3-{3-oxo-2H,3H,4H-pyrido[3,2-b][1,4]oxazin-6-yl}-octahydropyrrolo[3,4-e][1,3]oxazin-6-yl]ethyl}-2-oxo-1,2-dihydroquinoline-7-carbonitrile 12 (50 mg, 20%) LC-MS (Method A) 487.2 [M+H]⁺, RT 1.92 min. H NMR (Method C) (CDCl₃): δ ppm 8.25 (s, 1H), 7.75-7.65 (m, 3H), 7.49 (dd, J=8.6, 1.3 Hz, 1H), 7.39 (d, J=8.6 Hz, 1H), 7.30 (d, J=8.6 Hz, 1H), 6.82 (d, J=9.5 Hz, 1H), 4.66 (s, 2H), 4.52-4.32 (m, 3H), 4.23-4.20 (m, 1H), 3.64 (t, J=11.6 Hz, 1H), 3.34 (dd, J=9.4, 7.4 Hz, 1H), 3.25 (dd, J=9.4, 7.4 Hz, 1H), 3.11-2.96 (m, 3H), 2.84 (t, J=10.0 Hz, 1H), 2.60-2.55 (m, 1H).

### Example 13:—rel-(4aS*,7aR*-6-(2-{2-methoxy-7-oxo-7H,8H-pyrido[2,3-d]pyrimidin-8-yl}ethyl)-3-{3-oxo-2H,3H,4H-pyrido[3,2-b][1,4]oxazin-6-yl}-octahydropyrrolo[3,4-e][1,3]oxazin-2-one

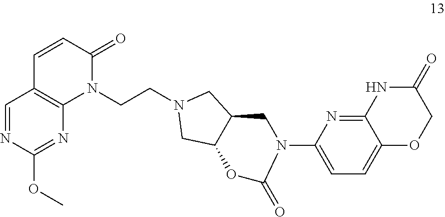 [SMILES: COc1ncc2ccc(=O)n(CCN3C[C@H]4CN(c5ccc6c(n5)NC(=O)CO6)C(=O)O[C@@H]4C3)c2n1]

### a) 8-(2,2-diethoxyethyl)-2-methoxy-7H,8H-pyrido[2,3-d]pyrimidin-7-one 13a

K₂CO₃ (143 mg, 1.04 mmol) was added to a suspension of 2-methoxy-7H,8H-pyrido[2,3-d]pyrimidin-7-one (prepared as described in J. Med. Chem., 54(22), 7834-7847; 2011) (100 mg, 0.56 mmol) in DMF (2 mL). The mixture was stirred at room temperature for 1 h. Bromoacetaldehyde diethyl acetal (0.09 mL, 0.62 mmol) was added and the mixture heated at 103° C. for 4 h. The reaction mixture was allowed to cool to room temperature then diluted with H₂O and extracted with DCM (3×20 mL). The combined organic extracts were washed with saturated brine (3 mL), dried over MgSO₄ and concentrated to afford a brown residue of 8-(2,2-diethoxyethyl)-2-methoxy-7H,8H-pyrido[2,3-d]pyrimidin-7-one 13a (91 mg, 55%) which was used without further purification.

### b) 2-(2-methoxy-7-oxo-7H,8H-pyrido[2,3-d]pyrimidin-8-yl)acetaldehyde 13b

A solution of TFA (6 mL, 1.02 mmol) in H₂O (1.5 mL) was added to 8-(2,2-diethoxyethyl)-2-methoxy-7H,8H-pyrido[2,3-d]pyrimidin-7-one 13a (300 mg, 1.02 mmol) and the reaction mixture stirred at room temperature for 3 h. The reaction was basified with saturated aqueous NaHCO₃ to pH 8. The mixture was extracted with DCM (20 mL) and IPA/DCM (1:3, 4×20 mL). The combined organic extracts were washed with brine, dried (MgSO₄), filtered, and concentrated under reduced pressure. The resulting residue was purified by chromatography eluting with 100% EtOAc to afford 2-(2-methoxy-7-oxo-7H,8H-pyrido[2,3-d]pyrimidin-8-yl)acetaldehyde 13b (173 mg, 77%) as a white solid. LC-MS (Method A) 220.4 [M+H]⁺, RT 1.39 min.

### c) rel-(4aS*,7aR*)-6-(2-{2-methoxy-7-oxo-7H,8H-pyrido[2,3-d]pyrimidin-8-yl}ethyl)-3-{4-[(4-methoxyphenyl)methyl]-3-oxo-2H,3H,4H-pyrido[3,2-b][1,4]oxazin-6-yl}-octahydropyrrolo[3,4-e][1,3]oxazin-2-one 13c

2-(2-methoxy-7-oxo-7H,8H-pyrido[2,3-d]pyrimidin-8-yl)acetaldehyde 13b (53 mg, 0.24 mmol), rel-6-[(4aS*,7aR*)-2-oxo-octahydropyrrolo[3,4-e][1,3]oxazin-3-yl]-4-[(4-methoxyphenyl)methyl]-2H,3H,4H-pyrido[3,2-b][1,4]oxazin-3-one 1c (100 mg, 0.24 mmol) and molecular sieves (3 Å) were added to DCM (20 mL) and stirred for 2 h. To this was added NaBH(OAc)₃ (77 mg, 0.37 mmol) and the mixture stirred for 2 h. The reaction was quenched with saturated aqueous K₂CO₃ (40 mL) and extracted with DCM (2×40 mL). The organic extracts were combined, dried (MgSO₄), filtered and concentrated under reduced pressure to yield rel-(4aS*,7aR*)-6-(2-{2-methoxy-7-oxo-7H,8H-pyrido[2,3-d]pyrimidin-8-yl}ethyl)-3-{4-[(4-methoxyphenyl)methyl]-3-oxo-2H,3H,4H-pyrido[3,2-b][1,4]oxazin-6-yl}-octahydropyrrolo[3,4-e][1,3]oxazin-2-one 13c (149 mg, 99%) as a brown oil. LC-MS (Method A) 614.2 [M+H]⁺, RT 2.51 min.

### d) rel-(4aS*,7aR*)-6-(2-{2-methoxy-7-oxo-7H,8H-pyrido[2,3-d]pyrimidin-8-yl}ethyl)-3-{3-oxo-2H,3H,4H-pyrido[3,2-b][1,4]oxazin-6-yl}-octahydropyrrolo[3,4-e][1,3]oxazin-2-one 13

To a stirred solution of rel-((4aS*,7aR*))-6-(2-{2-methoxy-7-oxo-7H,8H-pyrido[2,3-d]pyrimidin-8-yl}ethyl)-3-{4-[(4-methoxyphenyl)methyl]-3-oxo-2H,3H,4H-pyrido[3,2-b][1,4]oxazin-6-yl}-octahydropyrrolo[3,4-e][1,3]oxazin-2-one 13c (116 mg, 0.18 mmol) in DCM (5 mL), was added TFA (2 mL) and CF₃SO₃H (0.5 mL). Stirring was continued for 1 h at room temperature then MeOH (1 mL) added. Saturated aqueous Na₂CO₃ was added adjusting the pH between 8-9. The mixture was extracted with DCM (2×25 mL) and 10% MeOH/DCM (2×25 mL) and the combined organic extracts were passed through an Isolute SPE phase separator. The filtrate was concentrated under reduced pressure to afford a residue which was purified by silica gel chromatography using 0-20% EtOAc/MeOH to give rel-(4aS*,7aR*)-6-(2-{2-methoxy-7-oxo-7H,8H-pyrido[2,3-d]pyrimidin-8-yl}ethyl)-3-{3-oxo-2H,3H,4H-pyrido[3,2-b][1,4]oxazin-6-yl}-octahydropyrrolo[3,4-e][1,3]oxazin-2-one 13 (37 mg, 39%). LC-MS (Method A) 494.2 [M+H]⁺, RT 1.68 min. ¹H NMR (Method C) (DMSO-d6): δ ppm 11.26 (s, 1H), 8.93 (s, 1H), 7.96 (d, J=9.5 Hz, 1H), 7.41 (d, J=8.3 Hz, 1H), 7.16 (d, J=8.3 Hz, 1H), 6.58 (d, J=9.5 Hz, 1H), 4.68 (s, 2H), 4.68-4.61 (m, 1H), 4.50-4.61 (m, 1H), 4.30-4.45 (m, 1H), 4.10 (s, 3H), 4.10-4.00 (m, 1H), 3.54 (t, J=11.5 Hz, 1H), 3.18-3.14 (m, 2H), 3.02-2.91 (m, 1H), 2.88 (m, 2H), 1.24 (br s, 2H).

### Example 14:—rel-1-{3-[(4aS*,7aR*)-2-oxo-3-{3-oxo-2H,3H,4H-pyrido[3,2-b]1,4]oxazin-6-yl}-octahydropyrrolo[3,4-e][1,3]oxazin-6-yl]propyl}-2-oxo-1,2-dihydroquinoline-7-carbonitrile

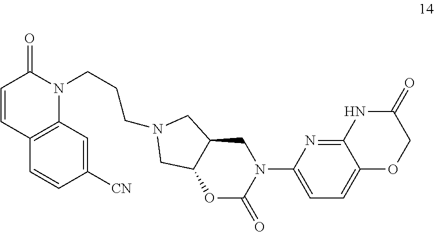 [SMILES: N#Cc1ccc2ccc(=O)n(CCCN3C[C@H]4CN(c5ccc6c(n5)NC(=O)CO6)C(=O)O[C@@H]4C3)c2c1]

### a) 7-bromo-1-(3,3-dimethoxypropyl)-1,2-dihydroquinolin-2-one 14a

A solution of 7-bromo-1,2-dihydroquinolin-2-one (1.5 g, 6.68 mmol), 3-bromo-1,1-dimethoxypropane (0.96 mL, 7.03 mmol) and cesium carbonate (6.54 g, 20.03 mmol) in DMF (100 mL) were heated for 17 h at 100° C. After cooling, H₂O (250 mL) was added and the mixture extracted with EtOAc (3×100 mL). The combined extracts were dried (MgSO₄), filtered and concentrated under reduced pressure. The residue was purified via silica gel chromatography using 0-100% EtOAc in pet. ether to give 7-bromo-1-(3,3-dimethoxypropyl)-1,2-dihydroquinolin-2-one 14a (740 mg, 34%) as a colourless solid. LC-MS (Method A) 296.0 [M-OMe]⁺, RT 2.78 min.

### b) 1-(3,3-dimethoxypropyl)-2-oxo-1,2-dihydroquinoline-7-carbonitrile 14b

A solution of 7-bromo-1-(3,3-dimethoxypropyl)-1,2-dihydroquinolin-2-one 14a (740 mg, 2.27 mmol), tetrakis(triphenylphosphine)palladium(0) (131 mg, 0.11 mmol) and zinc cyanide (159 mg, 1.4 mmol) in DMF (3 mL) in a sealed microwave vial was irradiated at 150° C. for 90 min in a Biotage Initator. After cooling, H₂O (50 mL) was added and the mixture extracted with EtOAc (3×30 mL). The combined extracts were dried (MgSO₄), filtered and concentrated under reduced pressure to give a yellow solid. The residue was purified via silica gel chromatography using (0-100% EtOAc in Pet ether) to give 1-(3,3-dimethoxypropyl)-2-oxo-1,2-dihydroquinoline-7-carbonitrile 14b (340 mg, 51%) as a yellow solid. LC-MS (Method A) 241.1 [M-OMe]⁺ RT 2.32 min.

### c) 2-oxo-1-(3-oxopropyl)-1,2-dihydroquinoline-7-Carbonitrile 14c

A solution 1-(3,3-dimethoxypropyl)-2-oxo-1,2-dihydroquinoline-7-carbonitrile 14b in 2M hydrogen chloride in THF (5 mL) was stirred at room temperature for 2 h. H₂O (20 mL) was added and the mixture extracted with EtOAc (2×20 mL). The combined extracts were dried (MgSO₄), filtered and concentrated under reduced pressure to give 2-oxo-1-(3-oxopropyl)-1,2-dihydroquinoline-7-Carbonitrile 14c (210 mg, 82%) as an off white solid. LC-MS (Method A) 227.5 [M+H]⁺, RT 1.47 min.

### d) rel-1-{3-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]propyl}-2-oxo-1,2-dihydroquinoline-7-carbonitrile 14d

Prepared using the method described in Example 13c using intermediates 2-oxo-1-(3-oxopropyl)-1,2-dihydroquinoline-7-Carbonitrile 14c (137 mg, 0.61 mmol) and rel-6-[(4aS*,7aR*)-2-oxo-octahydropyrrolo[3,4-e][1,3]oxazin-3-yl]-4-[(4-methoxyphenyl)methyl]-2H,3H,4H-pyrido[3,2-b][1,4]oxazin-3-one 1c (250 mg, 0.61 mmol) to give rel-1-{3-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]propyl}-2-oxo-1,2-dihydroquinoline-7-carbonitrile 14d (370 mg, 96%) as a brown oil. LC-MS (Method A) 621.1 [M+H]⁺, RT 2.90 min.

### e) rel-1-{3-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]propyl}-2-oxo-1,2-dihydroquinoline-7-carbonitrile

Prepared using the method described in Example 13d using rel-1-{3-[(4aS*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazin-6-yl]propyl}-2-oxo-1,2-dihydroquinoline-7-carbonitrile 14d (350 mg, 0.56 mmol). Purified via silica gel chromatography using 0-20% MeOH in EtOAc and dissolved in DCM (1 mL) and triturated with diethyl ether (5 mL) to a give rel-1-{3-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]propyl}-2-oxo-1,2-dihydroquinoline-7-carbonitrile 14 (60 mg, 22%) as a white solid. LC-MS (Method A) 501.1 [M+H]⁺; RT 2.03 min. ¹H NMR (Method C) (CDCl₃): δ ppm 7.94-7.91 (m, 1H), 7.70 (d, J=9.6 Hz, 1H), 7.66 (d, J=8.0 Hz, 1H), 7.46 (dd, J=7.9, 1.3 Hz, 1H), 7.42-7.38 (m, 1H), 7.30 (d, J=8.5 Hz, 1H), 6.83 (d, J=9.5 Hz, 1H), 4.66 (s, 2H), 4.56-4.47 (m, 1H), 4.42-4.29 (m, 2H), 4.21 (dd, J=11.2, 5.6 Hz, 1H), 3.66 (t, J=11.5 Hz, 1H), 3.21-3.16 (m, 2H), 2.99 (t, J=8.9 Hz, 1H), 2.82-2.74 (m, 3H), 2.68-2.55 (m, 1H), 1.96-1.90 (m, 2H).

### Example 15:—rel-1-{3-[(4aR*,7aR*)-2-oxo-3-{3-oxo-2H,3H,4H-pyrido[3,2-b][1,4]oxazin-6-yl}-octahydropyrrolo[3,4-e][1,3]oxazin-6-yl]propyl}-7-methoxy-1,2-dihydro-1,8-naphthyridin-2-one

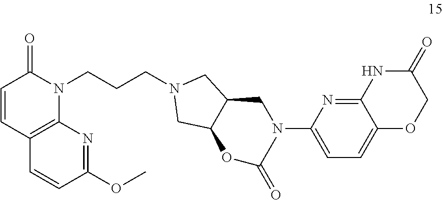 [SMILES: COc1ccc2ccc(=O)n(CCCN3C[C@@H](CNc4ccc5c(n4)NC(=O)CO5)[C@@H](OC(C)=O)C3)c2n1]

### a) rel-tert-butyl (4aR*,7aR*)-2-oxo-octahydropyrrolo[3,4-e][1,3]oxazine-6-carboxylate 15a

Prepared using the method described in Example 12a using rel-tert-butyl (3R*,4R*)-3-(aminomethyl)-4-hydroxypyrrolidine-1-carboxylate (2.91 g, 13.41 mmol) (WO2013171640 A1) affording rel-tert-butyl (4aR*,7aR*)-2-oxo-octahydropyrrolo[3,4-e][1,3]oxazine-6-carboxylate 15a (600 mg, 21%) as a white solid.

### b) rel-tert-butyl (4aR*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazine-6-carboxylate 15b

Prepared using the method described in Example 12b using rel-tert-butyl (4aR*,7aR*)-2-oxo-octahydropyrrolo[3,4-e][1,3]oxazine-6-carboxylate 15a (450 mg, 1.86 mmol) and 6-bromo-4-[(4-methoxyphenyl)methyl]-2H,3H,4H-pyrido[3,2-b][1,4]oxazin-3-one (prepared as described in WO2014108832) (650 mg, 1.86 mmol). Purified via silica gel chromatography using 0-100% EtOAc in petroleum ether to give rel-tert-butyl (4aR*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazine-6-carboxylate 15b (600 mg, 64%) as a brown oil. LC-MS (Method A) 511.1 [M+H]⁺, RT 3.03 min.

### c) rel-(4aR*,7aR*)-2-oxo-3-{3-oxo-2H,3H,4H-pyrido[3,2-b][1,4]oxazin-6-yl}-octahydropyrrolo[3,4-e][1,3]oxazin-6-ium trifluoroacetate 15c

Prepared using the method described in Example 12c using rel-tert-butyl (4aR*,7aR*)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazine-6-carboxylate 15b (600 mg, 1.17 mmol) to give rel-(4aR*,7aR*)-2-oxo-3-{3-oxo-2H,3H,4H-pyrido[3,2-b][1,4]oxazin-6-yl}-octahydropyrrolo[3,4-e][1,3]oxazin-6-ium trifluoroacetate 15c (475 mg, 99%). LC-MS (Method A) 291.1 [M+H]⁺, RT 1.17 min.

### d) 1-(3,3-dimethoxypropyl)-7-methoxy-1,2-dihydro-1,8-naphthyridin-2-one 15d

7-methoxy-1,2-dihydro-1,8-naphthyridin-2-one (0.9 g, 5.11 mmol), CsCO₃ (1.6 g, 4.91 mmol) and 3-bromo-1,1-dimethoxypropane (0.91 mL, 6.64 mmol) were suspended in DMF (50 mL) and heated to 90° C. for 1 h. H₂O (100 mL) was added and the mixture extracted with Et₂O (2×100 mL). Organics were combined, dried over MgSO₄ and solvent evaporated to afford an orange gum. Purification via column chromatography eluting with 50-100% Et₂O in heptane yielded 1-(3,3-dimethoxypropyl)-7-methoxy-1,2-dihydro-1,8-naphthyridin-2-one 15d (0.87 g, 2.97 mmol, 58%) as a yellow solid. LC-MS (Method A) 301.2 [M+Na]⁺, RT 2.45 min.

### e) 3-(7-methoxy-2-oxo-1,2-dihydro-1,8-naphthyridin-1-yl)propanal 15e

1-(3,3-dimethoxypropyl)-7-methoxy-1,2-dihydro-1,8-naphthyridin-2-one (0.64 g, 2.3 mmol) was added to THF (15 mL) and aqueous HCl (3M, 10 mL, 2.3 mmol) and the mixture was stirred at 60° C. for 27 h. The mixture was allowed to cool and saturated aqueous Na₂CO₃ (100 mL) was added then extracted with DCM (2×100 mL), dried over MgSO₄ and solvent evaporated under reduced pressure to afford 3-(7-methoxy-2-oxo-1,2-dihydro-1,8-naphthyridin-1-yl)propanal (320 mg, 1.24 mmol, 53.9% yield) as a yellow gum which solidified. ¹H NMR (Method C) ¹H-NMR (CDCl₃) δ: 9.78 (s 1H), 8.05 (d, J=8.3 Hz, 1H), 7.88 (d, J=9.5 Hz, 1H), 6.72 (d, J=8.3 Hz, 1H), 6.50 (d, J=9.5 Hz, 1H), 4.65 (t, J=7.3 Hz, 2H), 3.95 (s. 3H), 2.82 (t, J=7.3 Hz, 2H).

### f) rel-1-{3-[(4aR*,7aR*)-2-oxo-3-{3-oxo-2H,3H,4H-pyrido[3,2-b][1,4]oxazin-6-yl}-octahydropyrrolo[3,4-e][1,3]oxazin-6-yl]propyl}-7-methoxy-1,2-dihydro-1,8-naphthyridin-2-one 15

Prepared using the method described in Example 12d using rel-(4aR*,7aR*)-2-oxo-3-{3-oxo-2H,3H,4H-pyrido[3,2-b][1,4]oxazin-6-yl}-octahydropyrrolo[3,4-e][1,3]oxazin-6-ium trifluoroacetate, 15c (150 mg, 0.37 mmol) and 3-{2-methoxy-7-oxo-7H,8H-pyrido[2,3-d]pyrimidin-8-yl}propanal 15 e (77 mg, 0.33 mmol) Purified via silica gel chromatography using 0-20% MeOH in EtOAc, dissolved in DCM (1 mL) and triturated with diethyl ether to give rel-1-{3-[(4aR*,7aR*)-2-oxo-3-{3-oxo-2H,3H,4H-pyrido[3,2-b][1,4]oxazin-6-yl}-octahydropyrrolo[3,4-e][1,3]oxazin-6-yl]propyl}-7-methoxy-1,2-dihydro-1,8-naphthyridin-2-one 15 (40 mg, 22%) as a white solid. LC-MS (Method A) 507.2 [M+H]⁺, RT 2.33 min. ¹H NMR (Method C) (CDCl₃): δ ppm 8.42 (s, 1H), 7.72 (d, J=8.4 Hz, 1H), 7.57 (d, J=9.3 Hz, 1H), 7.43 (d, J=8.6 Hz, 1H), 7.21 (d, J=8.6 Hz, 1H), 6.62 (dd, J=8.9, 2.9 Hz, 2H), 4.92 (ddd, J=7.1, 5.0, 1.8 Hz, 1H), 4.61 (s, 2H), 4.55-4.48 (m, 2H), 4.01 (s, 3H), 3.99-3.91 (m, 2H), 3.12 (d, J=10.6 Hz, 1H), 3.07-3.05 (dd, J=9.3, 3.6 Hz, 1H), 2.88-2.85 (m, 1H), 2.75-2.69 (dt, J=11.8, 7.4 Hz, 1H), 2.64-2.61 (dd, J=10.9, 5.0 Hz, 1H), 2.54-2.44 (m, 2H), 2.09-1.95 (m, 1H), 1.94-1.84 (m, 1H).

### Example 16:—rel-1-{2-[(4aR*,7aR*)-2-oxo-3-{3-oxo-2H,3H,4H-pyrido[3,2-b][1,4]oxazin-6-yl}-octahydropyrrolo[3,4-e][1,3]oxazin-6-yl]ethyl}-7-methoxy-1,2-dihydro-1,8-naphthyridin-2-one

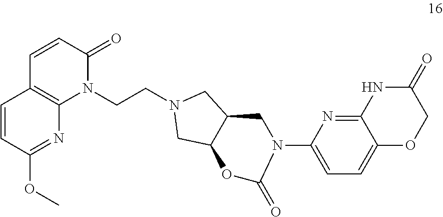 [SMILES: COc1ccc2ccc(=O)n(CCN3C[C@H]4CN(c5ccc6c(n5)NC(=O)CO6)C(=O)O[C@H]4C3)c2n1]

Prepared using the method described in Example 12d using rel- (4aR*,7aR*)-2-oxo-3-{3-oxo-2H,3H,4H-pyrido[3,2-b][1,4]oxazin-6-yl}-octahydropyrrolo[3,4-e][1,3]oxazin-6-ium trifluoroacetate 15c (200 mg, 0.49 mmol) and 2-(7-methoxy-2-oxo-1,2-dihydro-1,8-naphthyridin-1-yl)acetaldehyde (prepared as described in WO2008009700) (97 mg, 0.46 mmol). Purified via silica gel chromatography using 0-20% MeOH in EtOAc, dissolved in DCM (1 ml) and triturated with diethyl ether to give rel-1-{2-[(4aR*,7aR*)-2-oxo-3-{3-oxo-2H,3H,4H-pyrido[3,2-b][1,4]oxazin-6-yl}-octahydropyrrolo[3,4-e][1,3]oxazin-6-yl]ethyl}-7-methoxy-12-dihydro-1,8-naphthyridin-2-one 16 (18 mg, 7%) as a white solid. LC-MS (Method) 493.7 [M+H]⁺; RT 2.27 min. ¹H NMR (Method C) (CDCl₃): δ ppm 7.96 (s, 1H), 7.72 (d, J=8.3 Hz, 1H), 7.56 (d, J=9.3 Hz, 1H), 7.44 (d, J=8.3 Hz, 1H), 7.21 (s, 1H) 6.62 (d, J=8.3 Hz, 1H), 6.56 (d, J=9.3 Hz, 1H), 4.93 (dd, J=7.4, 5.0 Hz, 1H), 4.64 (s, 2H), 4.61 (t, J=7.2 Hz, 2H), 4.01 (s, 3H), 4.00-3.94 (m, 2H), 3.19 (d, J=10.9 Hz, 1H), 2.99-2.70 (m, 6H).

### Example 17:—1-{2-[(4aS,7aR)-2-oxo-3-{3-oxo-2H,3H,4H-pyrido[3,2-b][1,4]oxazin-6-yl}-octahydropyrrolo[3,4-e][1,3]oxazin-6-yl]ethyl}-2-oxo-1,2-dihydroquinoline-7-carbonitrile

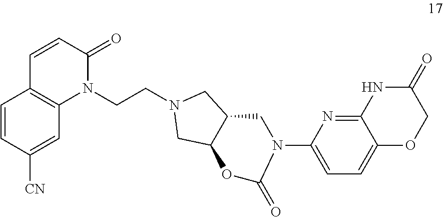 [SMILES: N#Cc1ccc2ccc(=O)n(CCN3C[C@@H]4CN(c5ccc6c(n5)NC(=O)CO6)C(=O)O[C@H]4C3)c2c1]

### a) tert-butyl (4aS,7aR)-2-oxo-octahydropyrrolo[3,4-e][1,3]oxazine-6-carboxylate 17a

Prepared using the method described in Example 12a using tert-butyl (3S,4R)-3-(aminomethyl)-4-hydroxypyrrolidine-1-carboxylate (3.3 g, 15.02 mmol) (preparation described in WO200809700 A1) to give tert-butyl (4aS,7aR)-2-oxo-octahydropyrrolo[3,4-e][1,3]oxazine-6-carboxylate 17a (900 mg, 25%) as a white solid.

### b) tert-butyl (4aS,7aR)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazine-6-carboxylate, 17b

Prepared using the method described in Example 12b using tert-butyl (4aS,7aR)-2-oxo-octahydropyrrolo[3,4-e][1,3]oxazine-6-carboxylate 17a (900 mg, 3.71 mmol) and 6-bromo-4-[(4-methoxyphenyl)methyl]-2H,3H,4H-pyrido[3,2-b][1,4]oxazin-3-one (preparation described in WO2014108832) (1.29 g and 3.71 mmol). Purified via silica gel chromatography using 0-100% EtOAc in pet ether to give tert-butyl (4aS,7aR)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazine-6-carboxylate 17b (600 mg, 37%) as a brown oil. LC-MS (Method A) 511.1 [M+H]⁺, RT 3.14 min.

### c) (4aS,7aR)-2-oxo-3-{3-oxo-2H,3H,4H-pyrido[3,2-b][1,4]oxazin-6-yl}-octahydropyrrolo[3,4-e][1,3]oxazin-6-ium trifluoroacetate, 17c

Prepared using the method described in Example 12c using tert-butyl (4aS,7aR)-3-{4-[(4-methoxyphenyl)methyl]-3-oxo-2H,3H,4H-pyrido[3,2-b][1,4]oxazin-6-yl}-2-oxo-octahydropyrrolo[3,4-e][1,3]oxazine-6-carboxylate 17b (600 mg, 1.17) to give (4aR,7aR)-2-oxo-3-{3-oxo-2H,3H,4H-pyrido[3,2-b][1,4]oxazin-6-yl}-octahydropyrrolo[3,4-e][1,3]oxazin-6-ium trifluoroacetate 17c (475 mg, 99%). LC-MS (Method) 291.1 [M+H]⁺, RT 1.17 min.

### d) 1-{2-[(4aS,7aR)-2-oxo-3-{3-oxo-2H,3H,4H-pyrido[3,2-b][1,4]oxazin-6-yl}-octahydropyrrolo[3,4-e][1,3]oxazin-6-yl]ethyl}-2-oxo-1,2-dihydroquinoline-7-carbonitrile 17

Prepared using the method described in Example 12d from (4aS,7aR)-2-oxo-3-{3-oxo-2H,3H,4H-pyrido[3,2-b][1,4]oxazin-6-yl}-octahydropyrrolo[3,4-e][1,3]oxazin-6-ium trifluoroacetate, 17c and 2-oxo-1-(2-oxoethyl)-1,2-dihydroquinoline-7-carbonitrile (93 mg, 0.47 mmol) (prepared as described in J. Med. Chem (2014) 57 (11), 4889-4905). Purified via silica gel chromatography using 0-20% MeOH in EtOAc then dissolved in DCM (1 mL) and triturated with Et₂O to give 1-{2-[(4aS,7aR)-2-oxo-3-{3-oxo-2H,3H,4H-pyrido[3,2-b][1,4]oxazin-6-yl}-octahydropyrrolo[3,4-e][1,3]oxazin-6-yl]ethyl}-2-oxo-1,2-dihydroquinoline-7-carbonitrile 17 (41 mg, 18%) as a white solid. LC-MS (Method A) 487.2 [M+H]⁺, RT 1.98 min. ¹H-NMR (Method C) (CDCl₃) ppm 8.50 (s, 1H), 7.75 (s, 1H), 7.69 (d, J=8.6 Hz, 2H), 7.49 (d, J=8.6 Hz, 1H), 7.38 (d, J=8.6 Hz, 1H), 7.30 (d, J=8.8 Hz, 1H), 6.81 (d, J=8.8 Hz, 1H), 4.66 (s, 2H), 4.51-4.34 (m, 3H), 4.21 (dd, J=11.6, 5.6 Hz, 1H), 3.65 (t, J=11.6 Hz, 1H), 3.36 (t, J=8.5 Hz, 1H), 3.26 (t, J=8.5 Hz, 1H), 3.12-2.97 (m, 3H), 2.86 (t, J=10.0 Hz, 1H), 2.64-2.55 (m, 1H).

### Example 18—Antibacterial Susceptibility Testing

Minimum Inhibitory Concentrations (MICs) versus planktonic bacteria are determined by the broth microdilution procedure according to the guidelines of the Clinical and Laboratory Standards Institute (Clinical and Laboratory Standards Institute. Methods for Dilution Antimicrobial Susceptibility Tests for Bacteria That Grow Aerobically; Approved Standard- Ninth Edition. CLSI document M07-A10, 2015). The broth dilution method involves a two-fold serial dilution of compounds in 96-well microtitre plates, giving a typical final concentration range of 0.25-128 μg/mL and a maximum final concentration of 1% DMSO.

The bacterial strains tested include the Gram-positive strains Staphylococcus aureus ATCC 29213 and Streptococcus pneumoniae ATCC 49619 and the Gram negative strains Acinetobacter baumannii NCTC 13420, Acinetobacter baumannii ATCC 19606, Enterobacter cloacae NCTC 13406, Escherichia coli ATCC 25922, E. coli ATCC BAA-2452, E. coli NCTC 13476, E. coli MG1655 and the gyrase A mutants E. coli MG1655 S83L and E. coli MG1655 D87G derived from the isogenic parent strain E. coli MG1655, Haemophilus influenzae ATCC 49247, Klebsiella pneumoniae ATCC 700603, Klebsiella pneumoniae NCTC 13443, Mycobacterium smegmatis ATCC 19420 (a recognised fast-growing and non-pathogenic surrogate for M. tuberculosis (Tuberculosis, 2010, 90:333)), Pseudomonas aeruginosa ATCC 27853.

Strains are grown in cation-adjusted Müller-Hinton broth at 37° C. in an ambient atmosphere. The MIC is determined as the lowest concentration of compound that inhibits growth following a 16-20 h incubation period. The data reported correspond to the modes of three independent experiments and is reported in Table 1.

In Table 1, an MIC (in μg/mL) of less or equal to 1 is assigned the letter A; a MIC of from 1 to 10 is assigned the letter B; a MIC of from 10 to 100 is assigned the letter C; and a MIC of over 100 is assigned the letter D.

All compounds tested show activity against both Gram-negative and Gram-positive bacteria.

TABLE 1
MIC values of reference compound and test compounds 1-17
against Gram-negative and Gram-positive bacterial strains

| Strains | CIP | 1 | 2 | 3 | 4 | 5 | 6 | 7 | 8 | 9 | 10 | 11 | 12 | 13 | 14 | 15 | 16 | 17 |
|---|---|---|---|---|---|---|---|---|---|---|---|---|---|---|---|---|---|---|
| A. baumannii NCTC 13420 | C | A | B | B | A | D | D | B | D | D | B | C | A | C | A | A | A | A |
| A. baumannii ATCC 19606 | A | | A | | | | | | | A | A | | B | | B | | A | B |
| E. cloacae NCTC 13406 | A | B | B | D | A | C | D | C | D | D | A | B | B | C | B | B | B | B |
| E. coli ATCC 25922 | A | A | B | B | A | C | B | B | C | C | A | B | A | | A | A | A | A |
| E. coli ATCC BAA 2452 | A | | D | | | | | | | | A | | A | | A | | | B |
| E. coli NCTC 13476 | C | | B | | | | | | | | B | | B | | B | | | B |
| E. coli MG1655 WT | A | | A | | | | | | | | A | | A | | | | | A |
| H. influenzae ATCC 49247 | A | A | A | B | | B | D | A | D | B | A | A | A | B | A | A | A | A |
| K. pneumoniae ATCC 700603 | A | C | C | D | B | D | D | C | D | D | B | C | C | C | B | C | C | C |
| K. pneumoniae NCTC 13443 | D | | | B | | | | | | | | | | | | | | |
| M. smegmatis ATCC 19420 | A | A | A | | | | | A | | A | | | | | | | | |
| P. aeruginosa ATCC 27853 | A | B | C | D | B | C | C | C | C | D | B | C | B | C | B | C | C | B |
| S. aureus ATCC 29213 | A | A | A | A | A | A | B | A | B | A | A | A | A | A | A | A | A | A |
| S. pneumoniae ATCC 49619 | A | A | A | B | | B | B | A | B | B | A | A | A | B | A | A | A | A |

TABLE 2
Fold increase in MIC against fluoroquinolone-resistant single
point mutant Escherichia coli strains (MG1655 S83L and MG1655
D87G) compared to the isogenic parent strain E. coli MG1655.

| | E. coli | E. coli |
|---|---|---|
| | MG1655 | MG1655 |
| | S83L¹ | D87G² |
| CIP | 16 | 8 |
| LEV | 8 | 8 |
| 2 | 1 | 1 |
| 10 | 1 | 1 |
| 12 | 1 | 1 |
| 14 | 4 | 2 |
| 17 | 0.5 | 2 |

CIP = ciprofloxacin;
LEV = levofloxacin
¹S83L mutation on DNA gyrase subunit GyrA
²D87G mutation on DNA gyrase subunit GyrA

Compounds 2, 10, 12 and 17 showed no significant (lower than or equal to 2-fold) loss of activity against the E. coli MG1655 fluoroquinolone-mutant strains. Thus compounds 2, 10, 12 and 17 were less susceptible to the gyrase S83L and D87G mutations than the fluoroquinolone antibiotics ciprofloxacin and levofloxacin.

### Example 19—Human Cell Viability Assay

Compounds are assessed for potential non-specific cytotoxic effects against a human hepatic cell line (HepG2 ATCC HB-8065). HepG2 cells are seeded at 20,000 cells/well in 96-well microtitre plates in minimal essential medium (MEM) supplemented with a final concentration of 10% FBS and 1 mM sodium pyruvate. After 24 h compound dilutions are prepared in Dulbecco's minimum essential media (DMEM) supplemented with final concentrations of 0.001% FBS, 0.3% bovine albumin and 0.02% HEPES and added to cells. Compounds are tested in two-fold serial dilutions over a final concentration range of 1-128 μg/mL in a final DMSO concentration of 1% vol/vol. Chlorpromazine is used as a positive control. Cells are incubated with compound at 37° C. and 5% CO₂ for a further 24 h, after which time the CellTiter-Glo reagent (Promega) is added. Luminescence is measured on a BMG Omega plate reader. Data are analysed using GraphPad Prism software to determine the concentration of compound that inhibits cell viability by fifty percent (IC₅₀). The results are provided in Table 3.

In Table 3, an IC₅₀ (in μg/mL) of less than 1 is assigned the letter D; an IC₅₀ of from 1 to 10 is assigned the letter C; an IC₅₀ of from 10 to 100 is assigned the letter B; and an IC₅₀ of over 100 is assigned the letter A.

TABLE 3
IC₅₀ values against HepG2 cell line

| | IC₅₀ (μg/mL) |
|---|---|
| Compound | HepG2 |
| 1 | A |
| 2 | B |
| 3 | B |
| 4 | A |
| 5 | A |
| 6 | A |
| 7 | A |
| 8 | A |
| 9 | C |
| 10 | A |
| 11 | B |
| 12 | A |
| 13 | C |
| 14 | B |
| 15 | A |
| 16 | A |
| 17 | A |

Compounds 1-17 show low toxicities against HepG2 human hepatic cell line. In particular, compounds 1, 4-8, 10, 12, and 15-17 showed no detectable toxicity against the tested human hepatic cell line. These compounds therefore show an excellent therapeutic benefit relative to their hepatic toxicities. Compounds 2, 3, 11, and 14 also demonstrate an acceptable level of hepatic toxicity relative to therapeutic activity. This indicates that these compounds have the potential to have an excellent therapeutic benefit relative to their hepatic toxicity.

1. A compound of formula (I), or a pharmaceutically acceptable salt or N-oxide thereof: 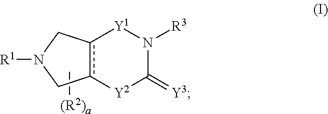
wherein [symbol] is a double bond or a single bond;
Y¹ is —CR⁴R⁴—;
Y² is independently selected from the group consisting of O and S;
Y³ is independently selected from the group consisting of O and S;
R¹ is independently selected from the group consisting of -L-Ar¹-Ar² and 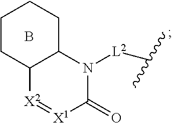
Ar¹ and Ar² are each independently a phenyl or monocyclic heteroaryl group;
-L¹- is —C₁-C₃-alkylene-;
X¹ is independently selected from the group consisting of N and CR and X² is independently selected from the group consisting of N and CR⁶; or
X¹ and X² together form a 5-membered heteroaryl ring;
-L²- is —C₂-C₃-alkylene-;
Ring B is independently selected from the group consisting of phenyl, monocyclic 6-membered heteroaryl and pyridinone, optionally substituted with a single —Y⁴—R⁷ group; Y⁴ is absent or is independently selected from the group consisting of NR⁸, O and S; where Ring B is a pyridinone ring, the nitrogen of the Ring B pyridinone may be attached to the proximal end of a —C₁-C₃-alkylene- group that is attached at its distal end to the group -L²-;
R² is independently at each occurrence selected from the group consisting of halo, nitro, cyano, NR⁹R¹⁰, NR⁹S(O)₂R⁹, NR⁹CONR⁹R⁹, NR⁹C(O)R⁹, NR⁹CO₂R⁹, OR⁹, SR⁹, SOR⁹, SO₃R⁹, SO₂R⁹, SO₂NR⁹R⁹, CO₂R⁹, C(O)R⁹, CONR⁹R⁹, C₁-C₄-alkyl, C₂-C₄-alkenyl, C₂-C₄-alkynyl, C₁-C₄-haloalkyl and O—C₁-C₄-haloalkyl;
R³ is a bicyclic carbocyclic or a bicyclic heterocyclic ring system in which at least one of the two rings is aryl or heteroaryl;
or R³ is -L³-phenyl; wherein -L³- is selected from the group consisting of —CR¹¹═CR¹¹— and —C₄-cycloalkyl-;
R⁴, R⁹ and R¹¹ are each independently at each occurrence selected from the group consisting of H and C₁-C₄-alkyl;
R⁵ and R⁶ are each independently selected from the group consisting of H, halo, cyano, C₁-C₄-alkyl and O—C₁-C₄-alkyl;
R⁷ is independently selected from the group consisting of H, C₁-C₄-alkyl, C₂-C₄-alkenyl, C₂-C₄-alkynyl, C₁-C₄-haloalkyl, C₃-C₈-cycloalkyl, ₄₋₇-heterocycloalkyl, phenyl, monocyclic heteroaryl and C₁-C₃-alkylene-R^{7a}; wherein R^{7a} is independently selected from the group consisting of C₃-C₈-cycloalkyl, ₄₋₇-heterocycloalkyl, phenyl and monocyclic heteroaryl;
R⁸ is independently selected from the group consisting of H and C₁-C₄-alkyl;
or R⁷ and R⁸ together with the nitrogen to which they are attached form a 4- to 7-membered heterocycloalkyl ring;
R¹⁰ is independently at each occurrence selected from the group consisting of H, C₁-C₄ alkyl, C₁-C₄-haloalkyl, S(O)₂—C₁-C₄-alkyl and C(O)—C₁-C₄-alkyl;
a is an integer from 0 to 4;
wherein any of the aforementioned alkyl, alkylene, alkenyl, alkynyl, haloalkyl, cycloalkyl, carbocyclic, heterocyclic, heterocycloalkyl, aryl, phenyl and heteroaryl groups is optionally substituted, where chemically possible, by 1 to 5 substituents which are each independently at each occurrence selected from the group consisting of oxo, ═NR^{a}, ═NOR^{a}, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl, C₁-C₄-haloalkyl and O—C₁-C₄-haloalkyl; wherein R^{a} is independently at each occurrence selected from the group consisting of H and C₁-C₄-alkyl.

2. The compound of claim 1, wherein the compound is represented by formula (VII):

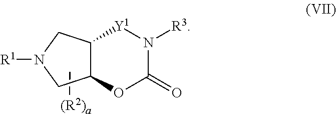

3. The compound of claim 1, wherein the compound is represented by formula (IX):

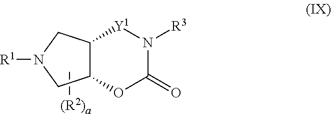

4. The compound of claim 1, wherein Y¹ is —CH₂—.

5. The compound of claim 1, wherein R¹ is: 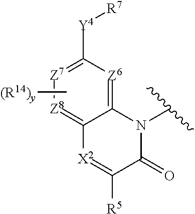
wherein y is an integer from 0 to 2; Z⁶, Z⁷ and Z⁸ are each independently carbon or nitrogen; provided that no more than 2 of Z⁶, Z⁷ and Z⁸ are nitrogen; and R¹⁴ is independently selected from the group consisting of halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}, SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl, C₁-C₄-haloalkyl and O—C₁-C₄-haloalkyl.

6. (canceled)

7. The compound of claim 5, wherein Y⁴ is O; and R⁷ is independently selected from the group consisting of C₁-C₄-alkyl, C₂-C₄-alkenyl, C₂-C₄-alkynyl, C₁-C₄-haloalkyl, C₃-C₈-cycloalkyl, ₄₋₇-heterocycloalkyl, phenyl, monocyclic heteroaryl and C₁-C₃-alkylene-R^{7a}.

8. The compound of claim 5, wherein R⁵ and R⁶, together with the carbons to which they are attached, form a 5-membered heteroaryl ring.

9. The compound of claim 8, wherein the heteroaryl ring is selected from the group consisting of oxazole, thiazole, isoxazole and isothiazole.

10. The compound of claim 1, wherein R¹ is -L¹-Ar¹-Ar²; wherein Ar¹ and Ar² are each independently a phenyl or monocyclic heteroaryl group.

11. The compound of claim 10, wherein Ar¹ is a phenyl group; and Ar² is a 6-membered heteroaryl group.

12. The compound of claim 10, wherein Ar¹ is a 6-membered heteroaryl group; and Ar² is a phenyl group.

13. (canceled)

14. The compound of claim 1, wherein R³ is: 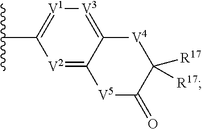
wherein V¹, V² and V³ are each independently selected from the group consisting of N and CR¹²;
with the proviso that no more than two of V¹, V² and V³ are N; V⁴ and V⁵ are each independently selected from the group consisting of O, S and NR^{a}; wherein R¹² is independently at each occurrence selected from the group consisting of H, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a} 2 SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄-haloalkyl; and R¹⁷ is independently at each occurrence selected from the group consisting of H, fluoro, cyano, CO₂R^{a}, C(O)R^{a}, CONR^{a}R^{a}, C₁-C₄-alkyl, C₂-C₄-alkenyl, C₂-C₄-alkynyl and C₁-C₄-haloalkyl.

15. (canceled)

16. The compound of claim 1, wherein the compound of formula (I) is selected from the group consisting of:
rel-1-{2-[(4aS*,7aR*)-2-oxo-3-{3-oxo-2H,3H,4H-pyrido[3,2-b][1,4]oxazin-6-yl}-octahydro-pyrrolo[3,4-e][1,3]oxazin-6-yl]ethyl}-7-methoxy-1,2-dihydro-1,8-naphthyridin-2-one;
rel-(4aS*,7aR*)-6-(3-{7-methoxy-4-oxo-4H,5H-[1,2]oxazolo[3,4-c]quinolin-5-yl}propyl)-3-{3-oxo-2H,3H,4H-pyrido[3,2-b][1,4]oxazin-6-yl}-octahydropyrrolo[3,4-e][1,3]oxazin-2-one;
rel-6-(3-{[(4aS*,7aR*)-2-oxo-3-{3-oxo-2H,3H,4H-pyrido[3,2-b][1,4]oxazin-6-yl}-octahydro-pyrrolo[3,4-e][1,3]oxazin-6-yl]methyl}-2-hydroxyphenyl)pyridine-2-carbonitrile;
rel-1-{3-[(4aS*,7aR*)-2-oxo-3-{3-oxo-2H,3H,4H-pyrido[3,2-b][1,4]oxazin-6-yl}-octahydro-pyrrolo[3,4-e][1,3]oxazin-6-yl]propyl}-1,2-dihydroquinolin-2-one;
rel-1-{2-[(4aS*,7aR*)-2-oxo-3-{3-oxo-2H,3H,4H-pyrido[3,2-b][1,4]oxazin-6-yl}-octahydro-pyrrolo[3,4-e][1,3]oxazin-6-yl]ethyl}-7-(2-hydroxyethoxy)-1,2-dihydro-1,8-naphthyridin-2-one;
rel-1-{3-[(4aS*,7aR*)-2-oxo-3-{3-oxo-2H,3H,4H-pyrido[3,2-b][1,4]oxazin-6-yl}-octahydro-pyrrolo[3,4-e][1,3]oxazin-6-yl]propyl}-7-(3-aminopropoxy)-1,2-dihydro-1,8-naphthyridin-2-one;
rel-1-{2-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]ethyl}-7-(pyridin-3-yloxy)-1,2-dihydro-1,8-naphthyridin-2-one;
rel-1-{2-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]ethyl}-7-(3-aminopropoxy)-1,2-dihydro-1,8-naphthyridin-2-one;
rel-(4aS*,7aR*)-6-(2-{7-methoxy-4-oxo-4H,5H-[1,2]oxazolo[3,4-c]quinolin-5-yl}ethyl)-3-{3-oxo-2H,3H,4H-pyrido[3,2-b][1,4]oxazin-6-yl}-octahydropyrrolo[3,4-e][1,3]oxazin-2-one;
rel-1-{2-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]ethyl}-2-oxo-1,2-dihydroquinoline-7-carbonitrile;
rel-(4aS*,7aR*)-6-(3-{2-methoxy-7-oxo-7H,8H-pyrido[2,3-d]pyrimidin-8-yl}propyl)-3-{3-oxo-2H,3H,4H-pyrido[3,2-b][1,4]oxazin-6-yl}-octahydropyrrolo[3,4-e][1,3]oxazin-2-one;
1-{12-[(4aR,7aS)-2-oxo-3-{3-oxo-2H,3H,4H-pyrido[3,2-b][1,4]oxazin-6-yl}-octahydropyrrolo[3,4-e][1,3]oxazin-6-yl]ethyl}-2-oxo-1,2-dihydroquinoline-7-carbonitrile;
rel-(4aS*,7aR*)-6-(2-{2-methoxy-7-oxo-7H,8H-pyrido[2,3-d]pyrimidin-8-yl}ethyl)-3-{3-oxo-2H,3H,4H-pyrido[3,2-b][1,4]oxazin-6-yl}-octahydropyrrolo[3,4-e][1,3]oxazin-2-one;
rel-1-{3-[(4aS*,7aR*)-2-oxo-3-{3-oxo-2H,3H,4H-pyrido[3,2-b][1,4]oxazin-6-yl}-octahydropyrrolo[3,4-e][1,3]oxazin-6-yl]propyl}-2-oxo-1,2-dihydroquinoline-7-carbonitrile;
rel-1-{3-[(4aR*,7aR*)-2-oxo-3-{3-oxo-2H,3H,4H-pyrido[3,2-b][1,4]oxazin-6-yl}-octahydropyrrolo[3,4-e][1,3]oxazin-6-yl]propyl}-7-methoxy-1,2-dihydro-1,8-naphthyridin-2-one;
rel-1-{2-[(4aR*,7aR*)-2-oxo-3-{3-oxo-2H,3H,4H-pyrido[3,2-b][1,4]oxazin-6-yl}-octahydropyrrolo[3,4-e][1,3]oxazin-6-yl]ethyl}-7-methoxy-1,2-dihydro-1,8-naphthyridin-2-one; and
1-{2-[(4aS,7aR)-2-oxo-3-{3-oxo-2H,3H,4H-pyrido[3,2-b][1,4]oxazin-6-yl}-octahydropyrrolo[3,4-e][1,3]oxazin-6-yl]ethyl}-2-oxo-1,2-dihydroquinoline-7-carbonitrile.

17-24. (canceled)

25. A pharmaceutical composition comprising a compound of claim 1; and at least one pharmaceutically acceptable excipient.

26. A method of treating a bacterial infection or a mycobacterial infection comprising administering to a patient in need thereof a therapeutic amount of a compound of formula (I), or a pharmaceutically acceptable salt or N-oxide thereof: 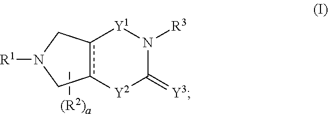
wherein [symbol] is a double bond or a single bond;
Y¹ is —CR⁴R⁴—;
Y² is independently selected from the group consisting of O and S;
Y³ is independently selected from the group consisting of O and S;
R¹ is independently selected from the group consisting of -L¹-Ar¹-Ar² and 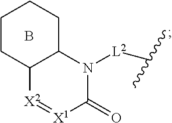
Ar¹ and Ar² are each independently a phenyl or monocyclic heteroaryl group;
-L¹- is —C₁-C₃-alkylene-;
X¹ is independently selected from the group consisting of N and CR⁵, and X² is independently selected from the group consisting of N and CR⁶; or
X¹ and X² together form a 5-membered heteroaryl ring;
-L²- is —C₂-C₃-alkylene-;
Ring B is independently selected from the group consisting of phenyl, monocyclic 6-membered heteroaryl and pyridinone, optionally substituted with a single —Y⁴—R⁷ group; Y⁴ is absent or is independently selected from the group consisting of NR⁸, O and S; where Ring B is a pyridinone ring, the nitrogen of the Ring B pyridinone may be attached to the proximal end of a —C₁-C₃-alkylene- group that is attached at its distal end to the group -L²;
R² is independently at each occurrence selected from the group consisting of halo, nitro, cyano, NR⁹R¹⁰, NR⁹S(O)₂R⁹, NR⁹CONR⁹R⁹, NR⁹C(O)R⁹, NR⁹CO₂R⁹, OR⁹, SR⁹, SOR⁹, SO₃R⁹, SO₂R⁹, SO₂NR⁹R⁹, CO₂R⁹, C(O)R⁹, CONR⁹R⁹, C₁-C₄-alkyl, C₂-C₄-alkenyl, C₂-C₄-alkynyl, C₁-C₄-haloalkyl and O—C₁-C₄-haloalkyl;
R³ is a bicyclic carbocyclic or a bicyclic heterocyclic ring system in which at least one of the two rings is aryl or heteroaryl;
or R³ is -L³-phenyl; wherein -L³- is selected from the group consisting of —CR¹¹═CR¹¹— and —C₄-cycloalkyl-;
R⁴, R⁹ and R¹¹ are each independently at each occurrence selected from the group consisting of H and C₁-C₄-alkyl;
R⁵ and R⁶ are each independently selected from the group consisting of H, halo, cyano, C₁-C₄-alkyl and O—C₁-C₄-alkyl;
R⁷ is independently selected from the group consisting of H, C₁-C₄-alkyl, C₂-C₄-alkenyl, C₂-C₄-alkynyl, C₁-C₄-haloalkyl, C₃-C₈-cycloalkyl, _{4_7}-heterocycloalkyl, phenyl, monocyclic heteroaryl and C₁-C₃-alkylene-R^{7a}; wherein R^{7a} is independently selected from the group consisting of C₃-C₈-cycloalkyl, _{4_7}-heterocycloalkyl, phenyl and monocyclic heteroaryl;
R⁸ is independently selected from the group consisting of H and C₁-C₄-alkyl;
or R⁷ and R⁸ together with the nitrogen to which they are attached form a 4- to 7-membered heterocycloalkyl ring;
R¹⁰ is independently at each occurrence selected from the group consisting of H, C₁-C₄ alkyl, C₁-C₄-haloalkyl, S(O)₂—C₁-C₄-alkyl and C(O)—C₁-C₄-alkyl;
a is an integer from 0 to 4;
wherein any of the aforementioned alkyl, alkylene, alkenyl, alkynyl, haloalkyl, cycloalkyl, carbocyclic, heterocyclic, heterocycloalkyl, aryl, phenyl and heteroaryl groups is optionally substituted, where chemically possible, by 1 to 5 substituents which are each independently at each occurrence selected from the group consisting of oxo, ═NR^{a}, ═NOR^{a}, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl, C₁-C₄-haloalkyl and O—C₁-C₄-haloalkyl; wherein R^{a} is independently at each occurrence selected from the group consisting of H and C₁-C₄-alkyl.

27. The method of claim 26, wherein the bacterial infection is caused by a Gram-positive bacteria.

28. The method of claim 26, wherein the bacterial infection is caused by a Gram-negative bacteria.

29. The method of claim 26, wherein the bacterial infection is caused by a bacterial strain that is resistant to at least one approved antibacterial drug.

30. The method of claim 26, wherein the mycobacterial infection is caused by mycobacteria.

31. The method of claim 26, wherein the mycobacterial infection is caused by a mycobacterial strain that is resistant to at least one approved antimycobacterial drug.
